(19) United States
(12) Patent Application Publication
(10) Pub. No.: US 2018/0372726 A1
(43) Pub. Date: Dec. 27, 2018

(54) INTEGRATIVE SINGLE-CELL AND CELL-FREE PLASMA RNA ANALYSIS

(71) Applicant: The Chinese University of Hong Kong, Shatin (HK)

(72) Inventors: Yuk-Ming Dennis Lo, Kowloon (CN); Cheuk Ho Tsang, Kowloon (CN); Peiyong Jiang, New Territories (CN); Lu Ji, New Territories (CN); Si Long Vong, New Territories (CN)

(21) Appl. No.: 15/981,683

(22) Filed: May 16, 2018

Related U.S. Application Data
(60) Provisional application No. 62/506,793, filed on May 16, 2017.

(51) Int. Cl. G01N 33/50; G06K 9/00; G06K 9/62

(52) U.S. Cl. CPC G01N 33/5091; G06K 9/00147; G01N 2800/60; G01N 2800/52; G01N 2800/56; G06K 9/6231

(57) ABSTRACT

Embodiments of the present technology involve integrative single-cell and cell-free plasma RNA transcriptomics. Embodiments allow for the determination of expressed regions that can be used to identify, determine, or diagnosis a condition or disorder in a subject. Methods described herein analyze cell-free RNA molecules for certain expressed regions. The specific expressed regions analyzed were previously determined to be indicative for a certain type of cell or grouping of cells. As a result, the amounts of cell-free reads at the specific expressed regions may be related to the number of cells in a tissue or organ. The number of cells in the tissue or organ may change as a result of cell death, metastasis, or other dynamics. A change in the number of cells in the tissue or organ may then be reflected in certain expressed regions in cell-free RNA.

### CROSS-REFERENCES TO RELATED APPLICATIONS

The present application claims priority from and is a non-provisional of U.S. Provisional Application No. 62/506,793 entitled “INTEGRATIVE SINGLE-CELL AND CELL-FREE PLASMA RNA ANALYSIS,” filed on May 16, 2017, the entire contents of which are herein incorporated by reference for all purposes.

### BACKGROUND

The health of an individual depends on the proper functioning and interaction of different organ systems in the body. Each organ system is composed of multicellular tissues that are specialized in achieving such purpose. In one estimation, the human body is composed of on average 37.2 trillion cells. Four basic tissue types—namely, epithelial, connective, nervous and muscular tissues—have been recognized in human. Human diseases originate from improper functioning or development of cells. In cancer, vulnerable cells acquire damaging genetic and epigenetic changes in the genome. Such changes results in change of gene expression and give rise to abnormal proliferation or other hallmarks of cancer cell behaviors.

In one example, one of the major function of the hematopoietic system is the maintenance of proper turnover of the blood tissue in circulation as a whole and the human blood contains different types of blood cells. Centrifugation can separate human whole blood into red blood cells (erythrocytes) and white blood cells (leukocytes). More detailed classification of different types of blood cells have been demonstrated through macro- or microscopic morphology of the cell, reactivity to certain types of histochemical or immunohistochemical staining, cellular response to certain types of external stimulation, characteristic cellular RNA expression profiles, or epigenetic modifications of the cellular DNA.

In another example, the human placenta is an essential organ during pregnancy to regulate maternal and fetal homeostasis. It is a discoid solid organ that is derived from the fetus and composed of multiple units of tree-like villous structure lined microscopically by uni- and multi-nucleated cells (trophoblasts), responsible for implantation into the maternal uterus and regulating the fetomaternal interface. Abnormal trophoblast implantation and development have been linked to potentially lethal hypertensive disorder during pregnancy, such as preeclampsia.

In another example, the liver is a major solid organ composed of functioning liver cells (hepatocytes), draining bile duct cells (cholangiocytes), and other connective types of cells specializing in metabolic function. Hepatitis B virus (HBV) is known to infect hepatocytes, integrate into hepatocyte genome in the liver and cause chronic hepatocyte cell death and inflammation (chronic hepatitis). Repeated reparative response to the hepatitis replaces hepatocytes with scar-forming cells (fibroblasts), thus liver cirrhosis. The accumulation of genetic mutations in the hepatocyte genome during prolonged cell death and regeneration results in malignant transformation of hepatocytes, i.e. hepatocellular carcinoma (HCC). HBV-related HCC accounts for ˜80% of the liver cancer in some localities, e.g. Hong Kong.

Detection of cellular abnormalities and the presence of disease in an organ system commonly requires direct tissue sampling (biopsy) of the organ of interest, which can carry infection and bleeding risk of invasive procedures. Non-invasive assessment by imaging, such as ultrasound scan, provides morphological and specific functional information of organ, such as blood flow. Liver ultrasonography has been employed in the screening of liver cancer in chronic HBV hepatitis patients and uterine artery Doppler analysis is used in preeclampsia prediction in early pregnancy. These however requires well-trained operators for assessment and does not assess the cellular aberrations directly.

Non-invasive methods of detecting cellular abnormalities and the presence of a disease in an organ system are desired. These and other improvements are addressed.

### BRIEF SUMMARY

Embodiments of the present technology involve integrative single-cell and cell-free plasma RNA transcriptomics. Embodiments allow for the determination of expressed regions that can be used to identify, determine, or diagnosis a condition or disorder in a subject. Methods described herein analyze cell-free RNA molecules for certain expressed regions. The specific expressed regions analyzed were previously determined to be indicative for a certain type of cell or grouping of cells. As a result, the amounts of cell-free reads at the specific expressed regions may be related to the number of cells in a tissue or organ. The number of cells in the tissue or organ may change as a result of cell death, metastasis, or other dynamics. A change in the number of cells in the tissue or organ may then be reflected in certain expressed regions in cell-free RNA.

Example methods in the present technology include analyzing reads from cellular RNA molecules obtained from a plurality of first subjects. The RNA molecules are grouped into clusters based on the regions preferentially expressed in each cluster and not in other clusters. These clusters may be associated with certain types of cells. Separately, cell-free RNA samples are obtained from a plurality of second subjects having different levels of a condition. The cell-free RNA samples are analyzed to determine one or more sets of one or more expressed regions that can be used to differentiate between different levels of the condition. The one or more sets of one or more expressed regions can then be used as an expressed marker for classifying future samples into different levels of the condition.

Analysis of cell-free RNA samples for expressed regions first determined through analysis of cells may provide a less noisy and more accurate method of determining the level of a condition of a subject. Because different types of cells may vary with the level of a condition, several expressed regions may be used to track the condition. The methods described herein can also provide a stronger signal compared to using a single genomic marker for the condition. In addition, methods described herein simplifies the screening process so that fewer expressed regions need to be analyzed for a correlation to the condition.

A better understanding of the nature and advantages of embodiments of the present invention may be gained with reference to the following detailed description and the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram explaining the integrative analysis of single-cell and plasma RNA transcriptomic in cellular dynamic monitoring and aberration discovery using pregnancy and preeclampsia as an example according to embodiments of the present invention.

FIG. 2 is a block flow diagram of a method of identifying an expressed marker to differentiate between different levels of a condition according to embodiments of the present invention.

FIG. 3 is a block flow diagram of a method of using a temporally-related sub-cohort in determining a level of condition according to embodiments of the present invention.

FIG. 4 is a table showing information for pregnant women used as subjects for analysis according to embodiments of the present invention.

FIG. 5 shows a computational single-cell transcriptomic clustering pattern of 20,518 placental cells by t-SNE analysis according to embodiments of the present invention.

FIG. 6 shows overlaying the expression of several genes resulting in clustered expression at defined groups of cells in the 2-dimensional projection according to embodiments of the present invention.

FIG. 7A shows the classification of fetal and maternal origin of each cluster in a dataset according to embodiments of the present invention.

FIG. 7B shows a column chart comparing the percentage of cells expressing Y-chromosome encoded genes in each cellular subgroup according to embodiments of the present invention.

FIG. 7C shows a biaxial scatter plot showing the distribution of cells of predicted fetal/maternal origin in the original t-SNE clustering distribution according to embodiments of the present invention.

FIG. 7D shows the expression pattern of stromal and myeloid markers in P5-7 subgroups according to embodiments of the present invention.

FIG. 7E shows t-SNE analysis with clustering of P5 cells with artificial P4/P7 duplets generated in silico according to embodiments of the present invention.

FIG. 7F shows biaxial scatter plots with the expression pattern of genes encoding for human leukocyte antigens among different subgroups of placental cells according to embodiments of the present invention.

FIG. 7G is a table summarizing the annotated nature of each cellular subgroup according to embodiments of the present invention.

FIG. 7H shows cellular subgroup composition heterogeneity in different single-cell transcriptomic datasets according to embodiments of the present invention.

FIG. 8 shows computational single-cell transcriptomic clustering pattern of placental cells and public peripheral blood mono-nucleated blood cells by t-SNE analysis according to embodiments of the present invention.

FIG. 9 is a table summarizing the annotated nature of different cell types in the merged PBMC and placental data according to embodiments of the present invention.

FIG. 10A shows a biaxial t-SNE plot showing the clustering pattern of peripheral blood mononucleated cells (PBMC) and placental cells according to embodiments of the present invention.

FIG. 10B shows a table summarizing the annotated nature of each cellular subgroups in the placenta/PBMC merged dataset according to embodiments of the present invention.

FIG. 10C shows biaxial scatter plots showing the expression pattern of specific marker genes among different subgroups of placental cells and PBMC according to embodiments of the present invention.

FIG. 10D is a heat map showing the average expression of cell-type specific signature genes in different PBMC and placental cells clusters according to embodiments of the present invention.

FIG. 10E shows box plots comparing the expression levels of different cell-type specific genes in human leukocytes, the liver, and the placenta according to embodiments of the present invention.

FIG. 10F shows cell signature analysis of the maternal plasma RNA profiles of a dataset in the literature according to embodiments of the present invention.

FIG. 11 shows the placental cellular dynamic in maternal plasma RNA profiles during pregnancy according to embodiments of the present invention.

FIG. 12A shows the extravillous trophoblast (EVTB) signature for preeclampsia according to embodiments of the present invention.

FIG. 12B shows cell death-related genes in the preeclampsia EVTB cluster according to embodiments of the present invention.

FIG. 13 shows signature scores for preeclampsia and control subjects for different cells according to embodiments of the present invention.

FIG. 14A shows the extravillous trophoblast (EVTB) signature for preeclampsia according to embodiments of the present invention.

FIG. 14B shows the single-cell transcriptome of placental biopsies from four preeclamptic patients and compared the intra-cluster transcriptomic heterogeneity in the HLA-G-expressing EVTB clusters between normal term and preeclamptic placentas according to embodiments of the present invention.

FIG. 15 shows the comparison of cell signature score levels of EVTB in maternal plasma samples from third trimester controls and severe early preeclampsia (PE) patients according to embodiments of the present invention.

FIG. 16 shows a list of genes for placental cells and PBMC according to embodiments of the present invention.

FIG. 17 is a heat map of the expression of a list of genes in placental cells and PBMC according to embodiments of the present invention.

FIG. 18 is a comparison of B cell-specific gene signature derived from single-cell transcriptomic analysis in plasma RNA between healthy control and patients with active SLE according to embodiments of the present invention.

FIG. 19 shows the sample name and the clinical conditions for the sample according to embodiments of the present invention.

FIG. 20 shows the expression pattern of selected genes that are known to be specific to certain types of cells in the human liver according to embodiments of the present invention.

FIG. 21 shows computational single-cell transcriptomic clustering pattern of HCC and adjacent non-tumor liver cells by PCA-t-SNE visualization according to embodiments of the present invention.

FIG. 22 shows identification of cell type-specific genes in the HCC/liver single-cell RNA transcriptomic dataset according to embodiments of the present invention.

FIG. 23 is a table listing cell type-specific genes for HCC/liver single-cell analysis according to embodiments of the present invention.

FIG. 24 shows a comparison of cell signature scores of different cell types in plasma for healthy controls, chronic HBV without cirrhosis, chronic HBV with cirrhosis and HCC pre-operation and HCC post-operation patients according to embodiments of the present invention.

FIG. 25 shows receiver operating characteristic curves of different approaches in the differentiation of non-HCC HBV (with or without cirrhosis) versus HBV-HCC patients according to embodiments of the present invention.

FIG. 26 shows the separation of a hepatocyte-like cell group into five subgroups by t-SNE analysis according to embodiments of the present invention.

FIG. 27 shows the origin of cells in the five subgroups of the hepatocyte-like cell group according to embodiments of the present invention.

FIG. 28 is an expression heat map showing the expression of preferentially expressed regions in the five subgroups of the hepatocyte-like cell group according to embodiments of the present invention.

FIG. 29 is a table of a list of genes preferentially expressed in a subgroup of the hepatocyte-like cell group according to embodiments of the present invention.

FIG. 30 illustrates a system according to embodiments of the present invention.

FIG. 31 shows a block diagram of an example computer system usable with system and methods according to embodiments of the present invention.

### TERMS

A “tissue” corresponds to a group of cells that group together as a functional unit. More than one type of cells can be found in a single tissue. Different types of tissue may consist of different types of cells (e.g., hepatocytes, alveolar cells or blood cells), but also may correspond to tissue from different organisms (mother vs. fetus) or to healthy cells vs. tumor cells.

A “biological sample” refers to any sample that is taken from a subject (e.g., a human, such as a pregnant woman, a person with cancer, or a person suspected of having cancer, an organ transplant recipient or a subject suspected of having a disease process involving an organ (e.g., the heart in myocardial infarction, or the brain in stroke, or the hematopoietic system in anemia) and contains one or more nucleic acid molecule(s) of interest. The biological sample can be a bodily fluid, such as blood, plasma, serum, urine, vaginal fluid, fluid from a hydrocele (e.g. of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g. thyroid, breast), etc. Stool samples can also be used. In various embodiments, the majority of DNA in a biological sample that has been enriched for cell-free DNA (e.g., a plasma sample obtained via a centrifugation protocol) can be cell-free, e.g., greater than 50%, 60%, 70%, 80%, 90%, 95%, or 99% of the DNA can be cell-free. The centrifugation protocol can include, for example, 3,000 g×10 minutes, obtaining the fluid part, and re-centrifuging at for example, 30,000 g for another 10 minutes to remove residual cells. The cell-free DNA in a sample can be derived from cells of various tissues, and thus the sample may include a mixture of cell-free DNA.

“Nucleic acid” may refer to deoxyribonucleotides or ribonucleotides and polymers thereof in either single- or double-stranded form. The term may encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs may include, without limitation, phosphorothioates, phosphoramidites, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The term “cutoff value” or amount as used in this disclosure means a numerical value or amount that is used to arbitrate between two or more states of classification—for example, whether a cell is similar to one type of cell. For example, if a parameter is greater than the cutoff value, the cell is not considered to be that type of cell, or if the parameter is less than the cutoff value, the cell is considered to be that type of cell or undetermined.

### DETAILED DESCRIPTION

Cells release cellular nucleic acid molecules (DNA or RNA) into the extracellular milieu passive or actively. These extracellular cell-free nucleic acid molecules can be detected in the circulating blood plasma. In pregnancy, it has been estimated that the fraction of fetal-derived RNA increases from only 3.7% in early pregnancy to 11.28% in late pregnancy (1, 2). As RNA transcription is cell-type specific, we reasoned that it is possible to infer cell-type specific changes and aberrations by analyzing the profile of multiple cell-free RNA transcripts in the plasma that are specific to the cell type of interest without directly sampling the tissues.

In the setting of pregnancy well-being assessment, several groups have explored the use of fetal-specific DNA polymorphisms, organ-specific DNA methylation (3), DNA fragmentation patterns (4, 5) and tissue-specific RNA transcripts (2) to isolate the placental contribution in the pool of circulating cell-free fetal nucleic acids and obtain overall changes of placental contribution. Nevertheless, these approaches are insufficient in examining the dynamic of the different fetal and maternal components in the placenta and differentiating the specific pathological changes of the placenta in different gestational pathologies at the cellular level.

One difficulty is the ascertainment of the origin of RNA transcripts. It has been shown that fetal RNA in maternal plasma is placenta-derived (6), and RNA transcripts believed to be derived from other non-placental fetal tissues have also been reported recently in maternal plasma (2). The tissue origins of these RNA transcripts are often inferred from comparison of whole tissue gene expression profiles of multiple tissues samples. As described above, biological tissues are composed of multiple types of cells originating from different developmental lineages. The expression profile from whole tissue therefore provide an averaged estimation of the population, distort the actual heterogeneous composition of the tissue and bias towards cells with the highest cell number in the tissue sample, such as trophoblast in the placenta. Previous studies have demonstrated that it is possible to dissect the cellular heterogeneity of complex biological organs based on single-cell transcriptomic RNA profiles and identified cell type-specific genes (7-10). It is therefore technically feasible to determine RNA expression profile of individual single cells of a representative tissue sample of the organ instead of assaying the tissue sample as a homogenized bulk.

It is unclear if the cellular heterogeneity information of the source tissue, for example the placenta in pregnancy, is retained in plasma RNA. If signals of different cell types of an organ of interest can be obtained through plasma RNA analysis, such signals can be quantified and analyzed separately or in combination to detect cellular pathology and diseases, for examples, of the placenta during pregnancy, or the organ harboring cancer, or the blood cells in autoimmune disease.

The biological properties and the degradation mechanism of cell-free circulating RNA in the plasma are different from that of cellular RNA, for example, plasma RNA is associated with filtratable substance in the plasma and may show a 5′ preponderance in certain transcripts (11, 12). The extrapolation of individual cell-type specific markers from tissues to plasma is not direct, for instance, fetal Rhesus D mRNA from fetal hematopoietic tissues cannot be easily detected in the plasma of Rhesus D-negative pregnant women, despite high expression levels in the fetal cord blood (13). In additions, it is known that the pool of cell-free circulating RNA is contributed from different tissue sources, and hematopoietic tissues and blood cells being the major component.

We developed an analytical approach to achieve this aim. We integrated single-cell transcriptomic RNA information of cellular heterogeneity into plasma RNA analysis, and derive a metrics for quantification and monitoring signals of different cellular components of complex organs in the cell-free plasma in autoimmune diseases, cancer, and prenatal conditions.

### I. GENERAL OVERVIEW

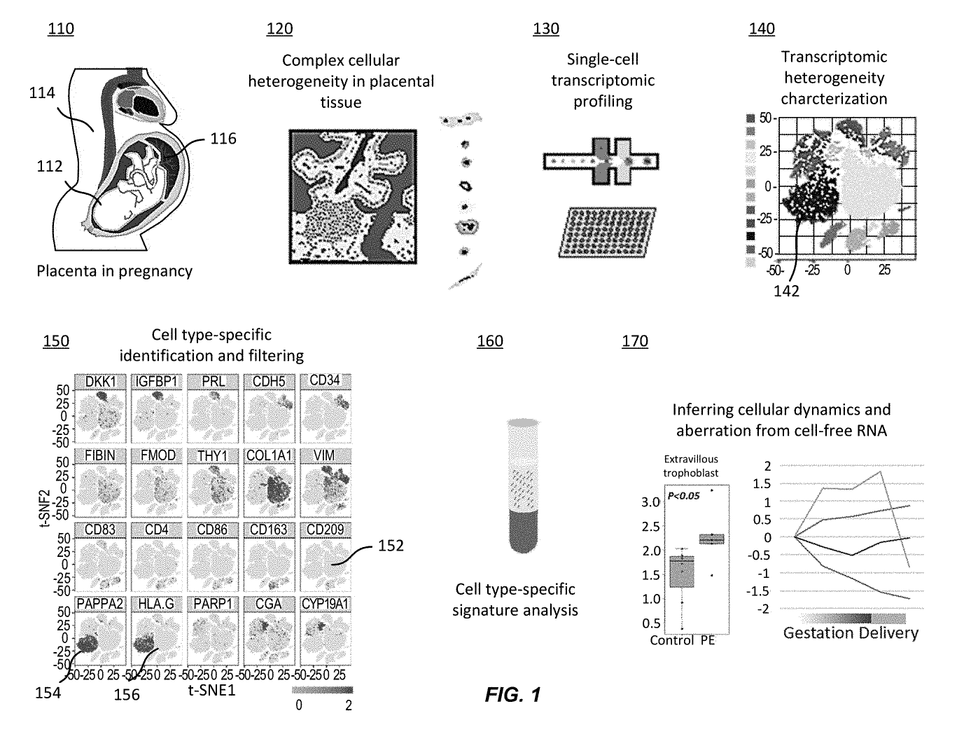

FIG. 1 is an illustration explaining the integrative analysis of single-cell and plasma RNA transcriptomic in cellular dynamic monitoring and aberration discovery using pregnancy and preeclampsia as an example. However, methods may be applied to autoimmune diseases, cancer, and other conditions. FIG. 1 provides a general overview of techniques. Additional details of the aspects and other embodiments are discussed later.

In diagram 110, a fetus 112 is shown in a pregnant female 114. Placenta 116 maintains the fetomaternal interface for gestational wellbeing.

Diagram 120 shows a portion of placenta 116 and shows that the organ is composed of multiple types of cells serving different functions. The source organ (placenta) tissue is dissociated into individual cells in this example. Preeclampsia is used as a condition in diagrams 110 and 120, but embodiments can be applied to other conditions, resulting in a similar procedure and illustrations. For example, diagram 110 may show a liver, and diagram 120 may show different cells in liver tissue.

A biopsy may be taken of the placenta or other organ of interest. The cells from the biopsy may then undergo transcriptomic profiling, e.g., after isolating individual cells. The transcriptomic profiling can determine expression levels for a plurality of genomic regions. The expression levels at these various regions can be used to identify clusters of cells that have similar expression levels at certain regions, e.g., regions that are preferentially expressed for a cluster.

Diagram 130 shows that single-cell transcriptomic profiles can be obtained by various technologies, such as microtiter plate-formatted chemistry or microfluidic droplet-based technology. Several biopsies may be taken so that cells are not limited to those from a single subject. In some instances, cells from a separate source (e.g., peripheral blood mononucleated cells [PBMC]) may also be obtained to merge with analysis of the cells from the biopsy. Single-cell RNA results may be obtained separately. The results may be merged using a computer system and then batch biases removed. In cancer, tissue cells with the tumor may be analyzed along with blood relevant cell lineage, such as lymphoid and myeloid cells.

Diagram 140 shows that placental cells can be grouped into different clusters based on transcriptional similarity (e.g., similar expression levels in preferentially expressed regions). The grouping into clusters may be based on a similar pattern of RNA reads from certain genes. The pattern may be based on absolute or relative (e.g., ranked) amounts of reads from the genes. For example, a certain cluster may have a first gene with the most number of reads and a second gene with the second most number of reads. As a further example, patterns could be several genes with similar expression levels (absolute amount, relative proportion, or relative ranks) uniquely present in a particular cluster or could be several genes having a unique order in terms of expression levels in a particular cluster.

The cells sharing similar patterns may be clustered together in 2D or higher dimensional space. For example, the Pearson's correlation coefficients between two cells based on all measurable genes in the single-cell transcriptomics data could be used for measuring the similarities of expression profiles. Other statistics also could be used, for example, Euclidean distance, squared Euclidean distance, Cosine similarity, Manhattan distance, maximum distance, minimum distance, Mahalanobis distance, or aforementioned distances adjusted by a set of weights. The grouping may be performed using principal component analysis (PCA) or other techniques described herein. Each cluster may correspond to a type of cell or a category of cells. If more than one source for the cells is used (e.g., placenta and PBMC), the cluster analysis may be performed on a merged data set.

In diagram 150, cell type-specific markers of each cell type are identified and filtered computationally by expression specificity to generate cell type-specific gene sets. Each panel in diagram 150, such as panels 152, 154, and 156, represents a specific gene. These genes may be known to be highly expressed in a particular type of cell. More red data points in each panel represent higher expression of a gene of interest. Thus, the genes corresponding to the relatively more red data points in comparison to other clusters suggest being more correlated with a specific cluster. The clusters in diagram 150 correspond to the identically positioned clusters in diagram 140. For example, the genes shown in panels 154 and 156 show a correlation with cluster 142 in diagram 140. The genes represented in panels 154 and 156 may be considered preferentially expressed regions for cluster 142.

The result of diagram 150 can be to identify a particular cluster in diagram 140 as corresponding to a particular type of cell. In this manner, the combination of the previous knowledge of a preferentially expressed region for a particular type of cell along with the clusters of cells having similar transcriptional profiles can be sued to identify new preferentially expressed regions for the cell type. In some embodiments, the original of the particular cell type (e.g., liver, fetal, etc.) does not need to be known, as the cells are still known to be of a same type. And, it may be sufficient to know that the preferentially expressed regions of the cell cluster provide sufficient discrimination power for different levels of a condition, when tested in later steps.

Diagram 160 shows that a cell-free sample, such as plasma, is tested following the determination of preferentially expressed regions for different clusters or cell types. A plurality of cell-free samples is tested from a plurality of subjects. The subjects can be grouped into cohorts having different levels of a condition. In the case of preeclampsia, the level of condition may be the severity of preeclampsia or simply the presence of preeclampsia. Expression of preferentially expressed genes in each cell-type were quantified and aggregated to calculate values of cell-type specific signatures in the plasma RNA profiles.

Diagram 170 shows that an overall value of the expression levels of certain genes can be used to monitor dynamic changes of the corresponding cellular component in the plasma serially (pregnancy progression in this example) or to identify cell-type specific aberrations (extravillous trophoblast in this example) between healthy pregnancy and patients suffering from specific diseases (preterm preeclampsia in this example). In diagram 170, the horizontal axis is gestational age, and the plot shows measurements for different cohorts, where a large separation at certain gestational ages illustrate that the expressed marker (set of preferentially expressed genes determined for a cluster of cells) can discriminate between the cohorts. Thus, such an expressed marker can be used to identify a subject that has a condition as opposed to not having the condition.

A. Example Method of Determining Expressed Markers

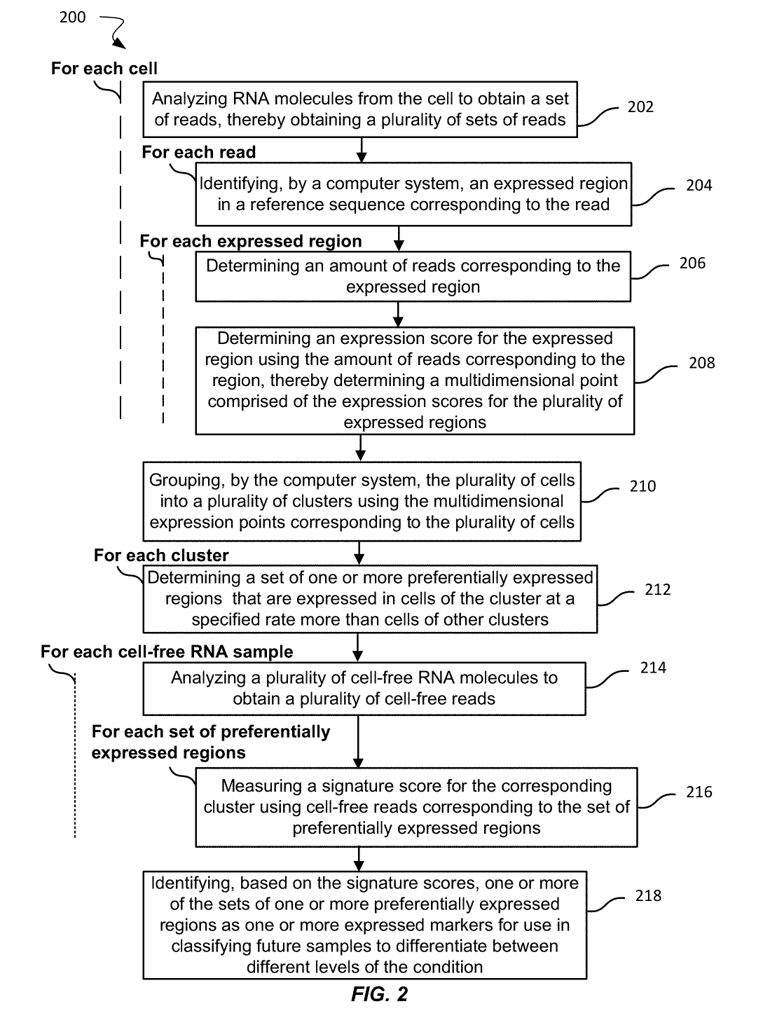

FIG. 2 shows an embodiment that includes a method 200 of identifying an express marker to differentiate between different levels of a condition. As examples, the level of the condition may be whether the condition exists, a severity of a condition, a stage of the condition, an outlook for the condition, the condition's response to treatment, or another measure of severity or progression of the condition.

The condition may be a pregnancy-associated condition. As examples, a pregnancy-associated condition may include preeclampsia, intrauterine growth restriction, invasive placentation, pre-term birth, hemolytic disease of the newborn, placental insufficiency, hydrops fetalis, fetal malformation, HELLP syndrome, systemic lupus erythematosus (SLE), or other immunological diseases of the mother. A pregnancy-associated condition may include a disorder characterized by abnormal relative expression levels of genes in maternal or fetal tissue. In some embodiments, the pregnancy-associated condition may be gestational age.

In other embodiments, the condition may include cancer. As examples, a cancer may include hepatocellular carcinoma, lung cancers, colorectal carcinoma, nasopharyngeal carcinoma, breast cancers, or any other cancers. The condition may include cancer in combination with a disorder, e.g., a hepatitis B infection. As examples, the level of cancer may be whether cancer exists, a stage of cancer (e.g., early stage and late stage), a size of tumor, the cancer's response to treatment, or another measure of a severity or progression of cancer. The condition may include an autoimmune disease, including systemic lupus erythematosus (SLE).

A sample including a plurality of cells may be obtained. Each cell of the plurality of cells may be isolated to enable the analyzing of the RNA molecules of a particular cell. The sample may be obtained with a biopsy. A placental tissue sample may be obtained by chorionic villus sampling (CVS), by amniocentesis, or from a placenta delivered full term. An organ tissue sample (e.g., for cancer) may be obtained with a surgical biopsy. Some samples may not involve incisions or cutting, e.g., obtaining blood (e.g., for a hematological cancer).

At block 202, RNA molecules from a cell is analyzed to obtain a set of reads. The analysis is repeated for each cell of a plurality of cells obtained from one or more first subjects, and therefore the analysis obtains a plurality of sets of reads. The analysis may be performed in various way, e.g., sequencing or using probes (e.g., fluorescent probes), as may be implemented using a microarray or PCR, or other example techniques provided herein. Such procedures can involve enrichment procedures, e.g., via amplification or capture.

The RNA molecules of each cell of the plurality of cells may be tagged with a unique code for the cell such that the associated reads include the unique code. In addition, for each cell of the plurality of cells, the set of reads associated with the unique code corresponding to the cell may be stored in the memory of a computer system. The computer system may be a specialized computer system for RNA analysis, including any computer system described herein.

If the condition is a pregnancy-associated condition, the first subjects may be female subjects each pregnant with a fetus. The plurality of cells may include placental cells, amnion cells, or chorion cells. If the condition is cancer, the first subjects may be subjects either with or without cancer, where the plurality of cells may include cells from various organs, e.g., including liver cells. If the condition is systemic lupus erythematosus (SLE), the first subjects may be subjects either with or without SLE, where the plurality of cells may include kidney cells, placental cells, or PBMC.

The set of reads may include sequence reads including those randomly obtained through massively parallel sequencing, including paired-end sequencing. The set of reads may also be obtained through reverse transcription PCR (RT-PCR), using probes to identify the presence of a certain region, digital PCR (droplet-based or well-based digital PCR), Western blotting, Northern blotting, fluorescent in situ hybridization (FISH), serial analysis of gene expression (SAGE), microarray, or sequencing.

At block 204, for each read of the sets of reads, an expressed region in a reference sequence corresponding to the read is identified by a computer system. The reference sequence may be a human reference transcriptome (e.g. data downloaded from UCSC refGene or de novo assembled transcripts) and/or a human reference genome (e.g. UCSC Hg19). Identifying an expressed region in a reference sequence is repeated for each read of the set of reads for each cell of the plurality of cells. Identifying the reference sequence corresponding to the read may include performing an alignment procedure using the read and a plurality of expressed regions of the reference sequence.

At block 206, for each of a plurality of expressed regions, an amount of reads corresponding to the expressed region is determined. Determining the amount of reads is also repeated for each of a plurality of expressed regions for each cell of the plurality of cells. As examples, the amount of reads may be the number of reads, a total length of reads, a percentage of reads, or a proportion of reads. The amount of reads may be the number of unique molecular identifiers (UMI). UMI is used to label the original RNA molecules.

Determining the amount of reads corresponding to a first expressed region of the first cell may use the unique code corresponding to the first cell so as to identify reads corresponding to the first cell so as to determine which reads correspond to a particular region, e.g., originate from that region, which may also be determined with probe-based techniques. Determining the amount of reads may also use results of the alignment procedure for the set of reads of the first cell. The unique code may be a barcode that is sequenced with the actual RNA sequence of the molecule. The barcode may differ from UMI in that the barcode is used to determine the cell, while UMI is used to label the original RNA molecule. Two RNA molecules from the same cell will have the same barcode but different UMI.

At block 208, for each of a plurality of expressed regions, an expression score for the expressed region is determined using the amount of sequence reads corresponding to the region. As a result, a multidimensional expression point including the expression scores for the plurality of expressed regions is determined. A multidimensional expression point for each cell may include the expression score in the cell for each expressed region. For example, the multidimensional expression point may be an array having the expression score of Gene 1, the expression score of Gene 2, the expression score of Gene 3, etc. Determining the expression score for the expressed region is also repeated for each of a plurality of expressed regions for each cell of a plurality of cells. Examples of expression scores are provided later, but may include absolute numbers of reads for a region, a proportional number of reads for a region, or other normalized amount of reads.

At block 210, the plurality of cells are grouped into a plurality of clusters using the multidimensional expression points corresponding to the plurality of cells. The plurality of clusters may be less than the plurality of cells. Grouping the plurality of cells into the plurality of clusters may include performing principal component analysis of the multidimensional expression points and performing dimensionality-reduction methods, such as principal component analysis (PCA) or diffusion maps, or by using force-based methods such as t-distributed stochastic neighbor embedding (t-SNE). The clusters may be determined using spatial parameters from a t-SNE or other plot. For example, a cluster may be determined where a minimum space exists between the cluster and another cluster in a plot. The grouping may be a result of the amounts of reads or a pattern of the amounts of reads for the expressed regions.

A cluster may be further grouped into sub-clusters or a subgroup. The cluster may be further divided because prior knowledge may indicate that sub-categories of cells exist. In addition, a statistical approach may be used to continue grouping of clusters, sub-clusters, etc. Grouping may continue until the variation within the cluster is minimized or reaches a target value. In addition, grouping may continue to achieve an optimal number of clusters to maximize average silhouette (Peter J. Rousseeuw (1987). “Silhouettes: a Graphical Aid to the Interpretation and Validation of Cluster Analysis.” Computational and Applied Mathematics. 20: 53-65) or the gap statistic (R. Tibshirani, G. Walther, and T. Hastie (Stanford University, 2001). http://web.stanford.edu/˜hastie/Papers/gap.pdf). The gap statistic is used to mean the deviation in intra-cluster variation between the reference data set with a random uniform distribution (computational simulation) and observed clusters.

At block 212, for each cluster of the plurality of clusters, a set of one or more preferentially expressed regions that are expressed in cells of the cluster at a specified rate more than cells of other clusters is determined. The specified rate may include a value determined from an average expression score for cells of the cluster and an average expression score for cells of other clusters. For example, the specified rate may be equal to a number of standard deviations (e.g., one, two, or three) for cells of other clusters. In other embodiments, the specified rate may be a z score, which describes the number of standard deviations that the average expression score for cells of the cluster is above the average expression score for cells of other clusters. In some embodiments, the specified rate may be a certain percentage over the average expression score for cells of other clusters. The specified rate may represent a cutoff or threshold to indicate a statistical difference from the average expression score for cells of other clusters.

The first cluster of the plurality of clusters may be identified to include a first type of cell by comparing the set of one or more preferentially expressed regions of the first cluster with one or more regions known to be preferentially expressed in the first type of cell. For example, a stromal cell may be known to preferentially express a certain region. A cluster with at least that region in the set of one or more preferentially expressed regions could then be deduced to be a stromal cell. The association of the cluster with a type of cell may be based on more than one preferentially expressed region. In some embodiments, a cluster may not be associated with a type of cell, as the identification of the type of cell may not be used for further analysis.

Example types of cells may include decidual, endothelial, vascular smooth muscle, stromal, dendritic, Hofbauer, T, erythroblast, extravillous trophobast, cytotrophoblast, syncytiotrophoblast, B, monocyte, hepatocyte-like, cholangiocyte-like, myofibroblast-like, endothelial, lymphoid, or myeloid cells.

At block 214, the plurality of cell-free RNA molecules is analyzed to obtain a plurality of cell-free reads. The analysis is repeated for each cell-free RNA sample of a plurality of cell-free RNA samples. The plurality of cell-free RNA samples are from a plurality of cohorts of second subjects. Each cohort of the plurality of cohorts may have a different level of the condition. For example, the plurality of cohorts may include a cohort without the condition, a cohort with the condition at an early stage, a cohort with the condition at a mid-stage,

The cohorts may have sub-cohorts that describe other characteristics of the second subjects. For example, a sub-cohort may be have the same temporal aspect related to the condition or the second subject. The sub-cohort may be a duration of the condition, a duration of treatment for the condition, time since diagnosis, or post-operative survival time. In some embodiments, a sub-cohort may have the same gender, same ethnicity, same geographic location, same age, or other same characteristic of the second subject.

The cell-free RNA samples may be obtained from plasma or serum (or other biological samples including cell-free RNA) of the second subjects. The second subjects may be the same subjects as the first subjects. However, in some embodiments, the second subjects may be different from the first subjects. In other embodiments, some subjects of the second subjects are the same as the first subjects, while some subjects of the second subjects are different from the remainder of the first subjects.

If the condition is a pregnancy-associated condition, the second subjects may be female subjects each pregnant with a fetus. Each cohort may include sub-cohorts that have different gestational ages for the same level of condition associated with the cohort. A sub-cohort may also include similar age of the female subject, similar age of the father of the fetus, or similar lifestyle of the female subject.

If the condition is cancer, the second subjects may include subjects with a tumor and may optionally include subjects without a tumor. The sub-cohort for cancer may be subjects with cancer showing similar molecular positivity (e.g. breast cancer with HER2 positive sub-cohort). In some embodiments, the sub-cohort could be subjects with cancer accompanied by other clinical complications, such as diabetes. A sub-cohort may have similar age, gender, tumor anatomical structures, metastasis status, or lifestyle.

At block 216, for each set of one or more preferentially expressed regions of the plurality of sets of one or more preferentially expressed regions, a signature score is measured for the corresponding cluster using cell-free reads corresponding to the set of one or more preferentially expressed regions. The measurement is repeated for each set of one or more preferentially expressed regions for each cell-free RNA sample of the plurality of cell-free RNA samples.

The signature score may be determined in various ways, e.g., as an average of an expression level for the one or more preferentially expressed regions for the corresponding cluster. The average may be the mean, median, or mode.

The signature score may be calculated from the following:

$$S = \frac{1}{n} {\sum }_{k = 1}^{n} log ( {E}_{k} + 1 )$$

where S is the signature score, n is the total number of cell-specific expressed regions in the set, and E is the expression level of the cell-specific expressed region.

At block 218, based on the signature scores, one or more of the sets of one or more preferentially expressed regions are identified as one or more expressed markers for use in classifying future samples to differentiate between different levels of the condition. An expressed marker refers to the set of one or more preferentially expressed regions collectively.

The preferentially expressed regions may be identified by identifying a signature score for a cohort and for a cluster that is statistically different than the signature scores for other cohorts in the cluster. For example, a preferentially expressed region for a cohort that has the condition may have a signature score statistically higher than the signature score for the preferentially expressed region for a cohort that does not have the condition. The statistical difference may be determined by setting a number of standard deviations the signature score is higher for the cohort than for other cohorts. The statistical difference may be determined by a t-test or another suitable statistical test.

All or a portion of the set of one or more preferentially expressed regions may be used as an expressed marker. A first set of one or more preferentially expressed regions may be a first expressed marker that differentiates between different levels of the condition for a first gestational age.

The first set of one or more preferentially expressed regions of a first cluster of the plurality of clusters may be a first expressed marker that differentiates between levels of cancer for a first tissue. The first cluster may include cells from the first tissue. The first tissue may be from the liver, and the first cluster may include liver cells. The tissue cells may include tumor cells and non-tumor cells, or in some embodiments, the cells may not include tumor cells. In some embodiments, the tissue cells may include normal cells and abnormal cells, which could be pathological. In embodiments, the first tissue may be from the lungs, throat, stomach, gall bladder, pancreas, intestines, colon, kidney, prostate, breast, bone, liver, blood cells (including T cells, B cells, neutrophils, monocytes, macrophage, megakaryocytes, thrombocytes, and natural killer cells), as well as bone marrow, spleen, colon, nasopharynx, esophagus, brain, or heart, and the first cluster may be cells from the corresponding tissue.

In some embodiments, the analysis of cells may include analysis of multiple types of cells. For example, placental cells may be analyzed for a set of one or more preferentially expressed regions. Additionally, PBMC may also be analyzed for another set of one or more preferentially expressed regions. As RNA molecules from both the placenta and PBMC may be present in a cell-free plasma sample, expressed markers in placenta and in PBMC can be identified in a cell-free sample for use in classifying future samples to differentiate between different levels of the condition. White blood cells may also be analyzed. Analyzing multiple types of cells in plasma may help understanding of tissue cellular dynamics in the plasma. For example, using PBMC or white blood cells may help elucidate the potential for blood cells shedding RNA into blood circulation. With more single-cell transcriptomics data available for more tissues (e.g., kidney, lung, colon, heart, brain, small intestine, bladder, testis, ovary, breast), the dynamics of plasma RNA with respect to cell origin may be better understood and monitored. Methods may also allow for associating cell-free RNA with types of cells. By understanding the increase and decrease of amounts of certain types of cells through cell-free RNA analysis, a greater understanding of the underlying condition and better understanding of how to treat the condition may be achieved.

Advantages of method 200 and other methods described herein include that the expressed markers can be identified more efficiently and accurately than other techniques. The methods described herein may allow for using multiple regions, instead of only one genomic marker, to differentiate between different levels of the condition. As a result, the method may be more robust to possible experimental error in measuring amounts from regions. A particular bulk tissue includes multiple subtypes of cells. For example, white blood cells include T cells, B cells, and neutrophils, etc., with neutrophils being the major population (>70%). Using a conventional way to determine the differentially expressed genes (e.g., genomic markers) between white blood cells and other tissues, the resulting markers would share similar patterns among T cells, B cells, and neutrophils and may not be unique to any type of blood cell. As a result, any changes seen in plasma RNA results may not effectively distinguish between type of blood cells, which would reduce sensitivity and accuracy in determining the level of a condition. For example, in a patient having B-cell lymphoma, the B cells would be expected to increase due to B cell proliferation. However, the conventional method would see the increased signal from white blood cells but could not inform the root source contributing to the increased signal. The conventional method would not be able to provide informative clues for diagnosis. But the single-cell RNA based marker allowed us to trace the dynamic changes directing to the cell of origins.

Embodiments also have an advantage distinguishing genes from a particular origin when the signal is low compared to the background. For example, the signal of a gene in a particular cell type of a tissue or organ (e.g. liver) may be weak in the circulating RNA molecules because of the overwhelming background of blood cell derived RNA as well as the other cell types in that tissue or organ. Using single cell RNA results, the methods are able to remove genes sharing the overlapping signals with the background and specifically aggregate the gene showing specific expression levels for the cell type associated with disease. For example, the ALB transcript is specific to liver according to RNA sequence data of liver tissue in comparison with blood cells. However, ALB expression levels cannot be used for distinguishing between HCC subjects and HBV carriers due to the ALB expression levels lacking specificity in tumor cells compared with background liver cells and the weak signal of single marker. With the use of single cell RNA sequencing approach, we can uncover the tumor cell specific transcripts with respect to background hepatic cells and aggregate more markers to increase the single to noise ratio, as evidenced by the receiver operating characteristic (ROC) curves described later in this document.

B. Example Methods of Determining Level of Condition in a Subject

The method may include determining the level of a condition in a third subject. The third subject may be a subject different than any subject included in the first subjects or the second subjects. The method may further include receiving a plurality of cell-free reads from an analysis of cell-free RNA molecules from a biological sample obtained from a third subject. In some embodiments, the plurality of cell-free RNA molecules from the biological sample obtained from the third subject may be analyzed to obtain the plurality of cell-free reads. The analysis of the cell-free RNA molecules may be by any suitable process described herein. For each preferentially expressed region of a first expressed marker, an amount of reads for the preferentially expressed region is determined. The amount of reads may be any amount described herein.

The amount of reads for one or more preferentially expressed regions is compared to one or more reference values. The comparison may include comparing the amount of reads for each preferentially expressed region to a reference value for each preferentially expressed region. The total number of preferentially expressed regions where the amount of reads exceeds the reference value may then be used in the comparison and may need to meet or exceed a certain number or percentage. For example, the total number of preferentially expressed regions where the amount of reads exceeds the corresponding reference value may meet or exceed 50%, 60%, 70%, 80%, 90%, or 100% of the number of preferentially expressed regions in an expressed marker in order to determine that the level of the condition. In some embodiments, the comparison may include calculating an overall score from the amount of reads for one or more preferentially expressed regions, and comparing the overall score to one reference value. The overall score may be calculated from summing the amounts of reads for a plurality of the preferentially expressed regions, which may include all the preferentially expressed regions of the expressed marker. The level of the condition may be determined if the overall score exceeds the reference value.

The one or more reference values may be previously determined from previously tested subjects, including the plurality of second subjects. The reference values may be based on an average value for a subject without the condition, and the reference value may be a cutoff that indicates a statistically different value. For example, the reference value may be one, two, or three standard deviations exceeding the average amount of reads for a preferentially expressed region.

Based on the comparisons of the amount of reads for one or more preferentially expressed regions to one or more reference values, the level of the condition for the third subject is determined. The separation between the amount of reads to the one or more reference values may indicate a confidence in the determination of the level of the condition. For example, an amount of reads that is just greater than a reference value may indicate a lower confidence or probability of the level of condition compared to when the amount of reads is much greater than the reference value.

In some embodiments, a plurality of expressed markers may be used for an equal plurality of levels of the condition. The amount of reads for the sets of preferentially expressed regions may be compared to reference values appropriate to each level of the plurality of levels of the condition. In some cases, the amounts of reads may exceed the reference values for multiple levels of the condition. The level of condition may be determined based on how much the reference value or values are exceeded at each level. The level where the reference value is exceeded by the most may be determined to be the level of the condition.

The method may further include treating the third subject for the condition. If the condition is preeclampsia, the treatment may include increased frequency of prenatal physician visits, bed rest, or induced delivery. If the condition is cancer, the treatment may include surgery, radiation therapy, chemotherapy, immunotherapy, targeted therapy, hormone therapy, stem cell transplant, or precision medicine.

In some embodiments, determining the level of a condition in a third subject may be performed separately from the method for identifying the one or more expressed markers. For example, the one or more expressed markers may be provided or known. A biological sample including cell-free RNA molecules from the third subject can then be analyzed as described above to determine the level of condition for the third subject.

C. Example Method Using Temporal Information to Select Expressed Markers

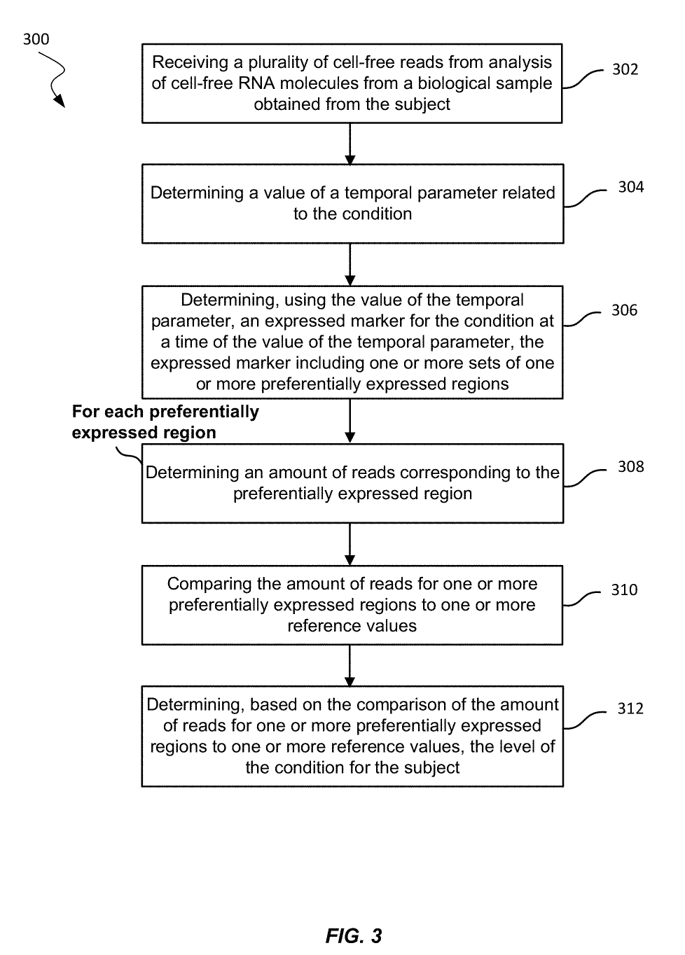

As described above, a sub-cohort may be characterized as having the same temporal aspect related to the condition or the second subject. FIG. 3 shows a method 300 of using a temporally-related sub-cohort in determining the level of a condition in a subject. The condition may include a pregnancy-associated condition, preeclampsia, cancer, SLE, or any other condition described herein.

At block 302, a plurality of cell-free reads from analysis of cell-free RNA molecules from a biological sample obtained from the subject is received. The plurality of cell-free reads may be received in any manner described herein. The method may further include obtaining a biological sample including the cell-free RNA molecules and then analyzing the cell-free RNA molecules to obtain the cell-free reads, as described herein.

At block 304, a value of a temporal parameter related to the condition is determined. If the condition is a pregnancy-associated condition, then the temporal parameter may be gestational age. The gestational age may be expressed as a week of pregnancy, a month of pregnancy, or a trimester of pregnancy. If the condition is cancer, then the temporal parameter may be a duration of treatment for cancer, a time since the diagnosis of cancer, or post-operative survival time.

At block 306, an expressed marker for the condition at a time of the value of the temporal parameter is determined using the value of the temporal parameter. The expressed marker include one or more sets of preferentially expressed regions. The determination may include analyzing expressed regions for regions that are not only preferentially expressed for the level of condition, but further analyzing the expressed regions for ones that are preferentially expressed at or near the value of the temporal parameter. In other words, the determination of the expressed markers may use the sub-cohorts described above. The preferential expression of a region may depend on the particular sub-cohort or sub-cohorts. For example, for a pregnancy-associated condition, a region may be preferentially expressed in the first trimester but not in the third trimester.

At block 308, for each preferentially expressed region of the expressed marker, an amount of reads corresponding to the preferentially expressed region is determined. The amount of reads may be any amount described herein. The amount of reads may be determined by aligning to the preferentially expressed region.

At block 310, the amount of reads for one or more preferentially expressed regions may be compared to one or more reference values. As described above, the comparison may include comparing amounts for each preferentially expressed region to a corresponding reference value for the preferentially expressed region, or the comparison may include an overall score for amounts from multiple expressed regions to a single reference value. The comparison may include any comparison technique described herein.

At block 312, based on the comparison of the amount of reads for one or more preferentially expressed regions to one or more reference values, the level of the condition for the subject is determined. As example, the level of the condition may be whether the condition exists, a severity of a condition, a stage of the condition, an outlook for the condition, the condition's response to treatment, or another measure of severity or progression of the condition. The method may further include a confidence level or probability for the level of the condition. The confidence may be based on a separation or ratio of the amounts of reads compared to the reference values. Based on the determined level of condition, a treatment plan can be developed to decrease the risk of harm to the subject. Methods may further include treating the subject according to the treatment plan.

### II. INTEGRATIVE SINGLE-CELL AND CELL-FREE PLASMA RNA ANALYSIS OF PLACENTA

Methods of determining a set of one or more preferentially expressed regions in cells and then identifying one or more of the sets of one or more preferentially expressed regions can be used with placental cells to determine the level of a pregnancy-associated condition.

The discovery of circulating cell-free fetal nucleic acids in maternal plasma has enabled the development of noninvasive prenatal diagnosis of fetal aneuploidy and monogenic diseases through detection of the pathogenic mutations, allelic and chromosomal imbalance (52, 53). Although it has been demonstrated that circulating cell-free fetal nucleic acids are placenta-derived, it remains difficult to study placental pathology using cell-free fetal nucleic acids and conventional bulk-tissue transcriptome profiling. One significant hurdle is the high cellular heterogeneity in the placenta, which cannot be addressed by total DNA quantitative analysis, targeted trophoblast-derived transcripts analysis or organ-specific transcripts monitoring. Previous studies have reported quantitative changes of multiple RNA transcripts during pregnancy (20, 21). However, there exists a gap in connecting the circulating pool of cell-free nucleic acids with their cellular origins. There is also a paucity of discussion of the cell-free nucleic acids dynamics of the non-trophoblastic component of the placenta during pregnancy. The advance in single-cell transcriptomic technology provided an opportunity for us to bridge the study of the placenta with circulating cell-free nucleic acids during pregnancy.

The placenta plays an essential role in the establishment of the utero-placental interface and the maintenance of fetal homeostasis during pregnancy (1). It is a genetically and developmentally heterogeneous organ composed of cells of maternal and fetal origins, from embryonic and extra-embryonic lineages. Histologically, the discoid human placenta is made up of multi-lobulated villous units. The human placenta exhibits a unique process of “controlled invasion” upon implantation. A distinct type of trophoblast cells, the extravillous trophoblast cells (EVTBs), migrate from the villi to infiltrate the maternal decidua during pregnancy. They participate in the remodeling of the uterine spiral arteries and interact with maternal lymphocytes to prevent allo-rejection of the fetus. Villous trophoblast cells, including multinucleated syncytiotrophoblasts (SCTBs) and villous cytotrophoblasts (VCTBs), lined the surface of the placental villi which are in direct contact with maternal blood. The entire placental villous structure is supported by stromal cells, resided by fetal macrophages (Hofbauer cells) and perfused by the fetal capillary vasculature.

Clinically, placental dysfunction has been linked to multiple major gestational complications such as preeclampsia toxemia (PET) (2). PET is a multi-system and potentially lethal gestational condition characterized by new onset of hypertension and proteinuria at ≥20 weeks of gestation. It affects 3-6% of pregnancies as a leading cause of maternal and perinatal morbidities. It can progress to systemic maternal disease with thrombocytopenia, liver derangement, renal failure, and seizure, resulting in significant fetal growth restriction or even fetal demise. Defective placental implantation and systemic vascular inflammation have been proposed as the major pathological mechanism in PET (2, 3).

In spite of the clinical significance of the placenta, direct placental tissue comparisons between patients with placental pathologies and healthy gestation-age matched controls is not feasible due to ethical concern of the invasiveness of direct placental biopsy. Instead, a number of clinical approaches, such as ultrasonographic imaging and maternal serum protein markers have been pursued to noninvasively monitor placental wellbeing during pregnancy (4, 5). Studies have shown that the placenta is the major source organ of circulating cell-free fetal nucleic acids in maternal plasma (6-8). Significantly elevated levels of total cell-free fetal DNA and selected placenta-specific RNA transcripts have also been reported in the maternal plasma of patients with PET (9-12) and preterm conditions (13-15), supporting a role for cell-free RNA in noninvasive monitoring of placental wellbeing. However, the overwhelming maternal hematopoietic background has created significant difficulties in detecting the placental signal (16). Previous studies have attempted to provide a more comprehensive assessment of maternal plasma nucleic acids by microarray analysis, massively parallel transcriptome or epigenome sequencing (17-23). Several groups have explored the use of fetal-specific DNA polymorphisms, organ-specific DNA methylation (22), DNA fragmentation patterns (24, 25) and organ-specific RNA transcripts (21) to isolate the placental contribution in the pool of circulating cell-free fetal nucleic acids and obtain overall changes of placental contribution. Nevertheless, it remains unknown if maternal plasma cell-free nucleic acid analysis can be used to dissect the dynamic and heterogeneous fetal and maternal placental components and resolve the complex changes of the placenta in different gestational pathologies at the cellular level.

We explored the use of droplet-based single-cell digital transcriptomic technology to comprehensively characterize the transcriptomic heterogeneity of the human placenta. We analyzed, in an unbiased manner, the single-cell transcriptomes of more than 24,000 non-markers selected placental cells from multiple normal and PET placentas. Using this comprehensive dataset, we successfully revealed the longitudinal cellular dynamics in maternal plasma during pregnancy progression and identified the potential cellular pathology noninvasively in preeclamptic placentas from maternal plasma cell-free RNA. Our study demonstrated the potential of an integrative and synergistic analytical approach of single-cell and plasma cell-free transcriptomic studies.

A. Dissection of the Cellular Heterogeneity of the Human Placenta

This section provides additional details to what was previously described for FIG. 1 for integrative analysis of single-cell and plasma RNA transcriptomic in cellular dynamic monitoring and aberration discovery using pregnancy and preeclampsia. We set out to obtain a comprehensive understanding of the cellular heterogeneity of the human placenta using large-scale droplet-based single-cell digital transcriptomic profiling (26) (FIG. 1). Other non-droplet based technologies allowing quantification of the RNA expression profile of individual cells with or without the need of tissue dissociation, such as transcript-counting by RNA in situ hybridization, single-cell RNA profiling by combinatorial barcoding, is also applicable in principle.

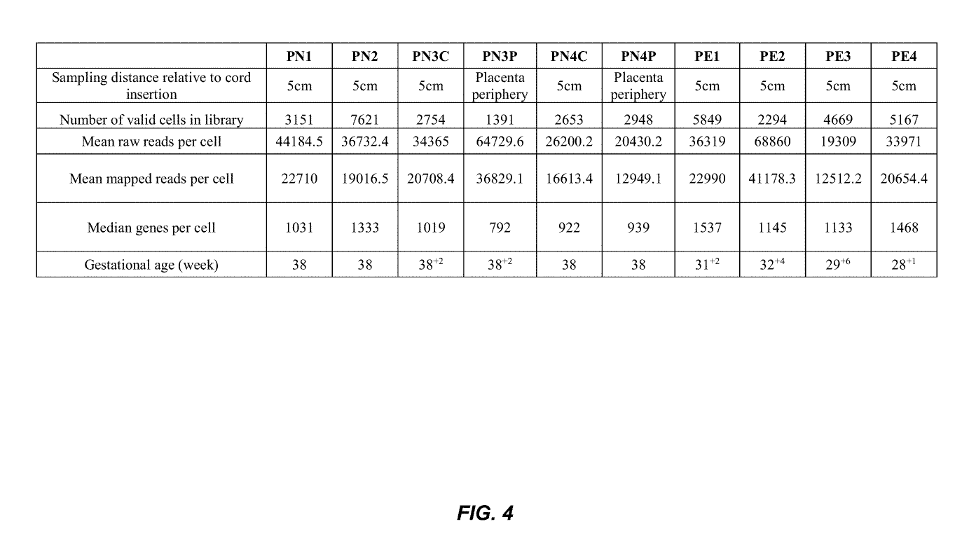

We collected biopsies at defined locations of multiple freshly cesarean section-delivered placentas (two male and two female babies) and dissociated the tissues into single-cell suspension without surface marker preselection. We obtained the single-cell transcriptome of 20,518 placental cells from six different placenta parenchymal biopsies. Obtaining the single-cell transcriptome of cells can be blocks 202 and 204 of FIG. 2. FIG. 4 shows information for six healthy pregnant women and four severely preeclamptic pregnant women who were subjects for the analysis. The average number of genes detected per libraries is 1,006 (792-1,333), with a mean coverage of 21,471 (16,613-36,829) reads per cell.

Clustering analysis by t-stochastic neighborhood embedding (t-SNE) identified 12 major clusters of placental cells in our dataset (P1-12). The clustering analysis was described with Diagram 140 in FIG. 1 and with block 210 of FIG. 2.

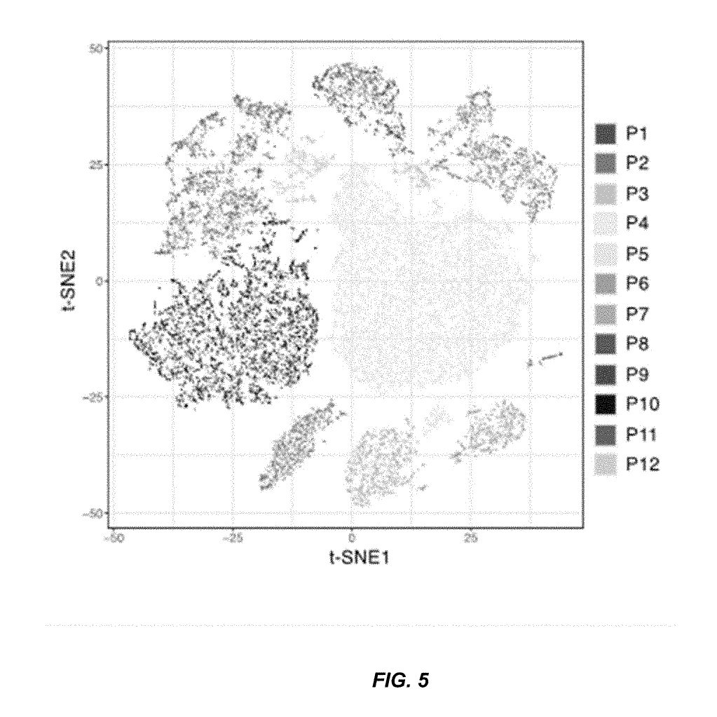

FIG. 5 shows the cellular heterogeneity of the placentas transcriptionally and the clustering in greater detail. Each dot in the plot represents the transcriptomic data from a single cell, the proximity of each dot is related to transcriptomic similarity. The clusters are further colored and grouped into subgroups (P1-12) based on spatial proximity in PCA-t-SNE and expression pattern of known cell type-specific marker expression from the literature.

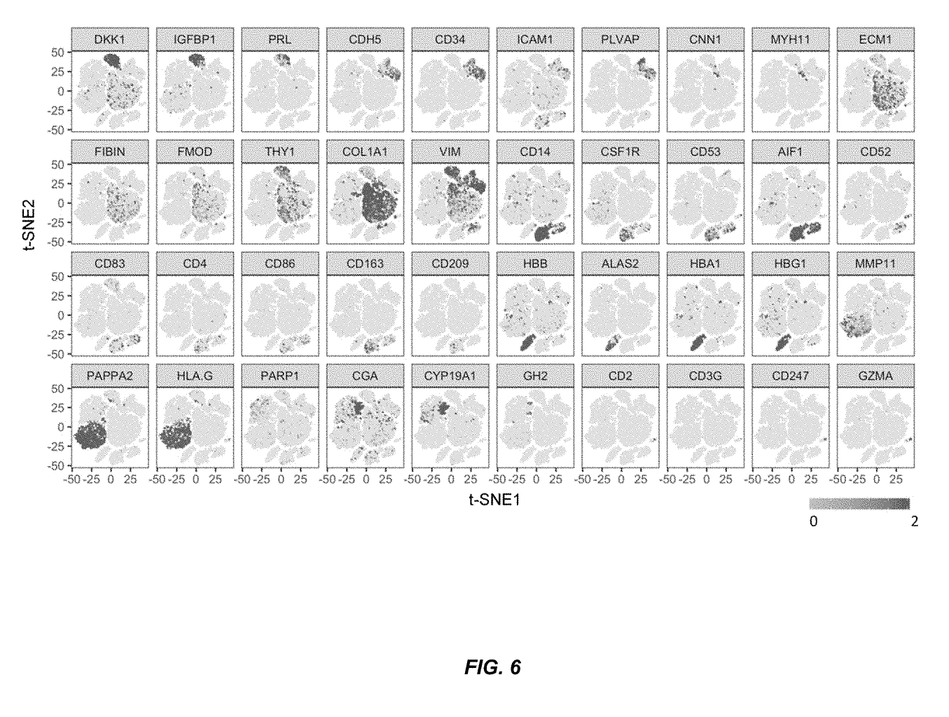

FIG. 6 shows overlaying the expression of several genes that are known in the literature to be specific to particular types of placental cells resulting in clustered expression at defined groups of cells in the 2-dimensional projection. Expression pattern of selected genes (titled in each box panel) that are known to be specific to certain types of cells in the human placenta (Expression quantified as log-transformed UMI counts at the range of 0-2). Each dot in the plot represents the transcriptomic data from a single cell. Grey color indicates no expression, and the brighter the shades of orange-red indicates the higher levels of expression.

The biological identity of the cell clusters can be directly inferred by the expression pattern of certain known cell type-specific genes. For example, CD34 genes are known to be specifically expressed in the endothelial cells of placental vessels, thus cells of the P2 clusters which showed high expression level of CD34 are likely endothelial cells.

In situation where the organ of interest is made up of cells from different genetic origin, for example, the placenta where maternal blood and decidual cells may be present in the placental biopsy and be detected in the single-cell RNA profile, genetic identity of the cell clusters can be inferred by exploiting the genetic differences between the cell origins present in the RNA transcripts.

Furthermore, we genotyped the genomewide SNP pattern of the mother and the fetus to differentiate the fetomaternal origin of individual cells genetically by comparing the ratio of fetal-to-maternal specific RNA SNPs in each subgroup and by examining the presence of Y chromosome-encoded transcripts in the cells from the placentas of male fetus-carrying pregnancies. The analysis of fetal and maternal origin is described in further detail below.

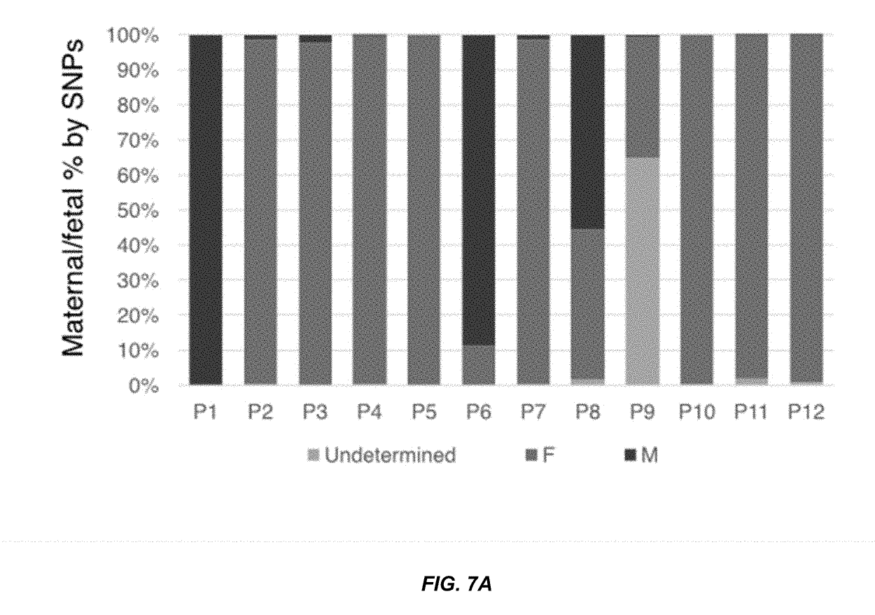

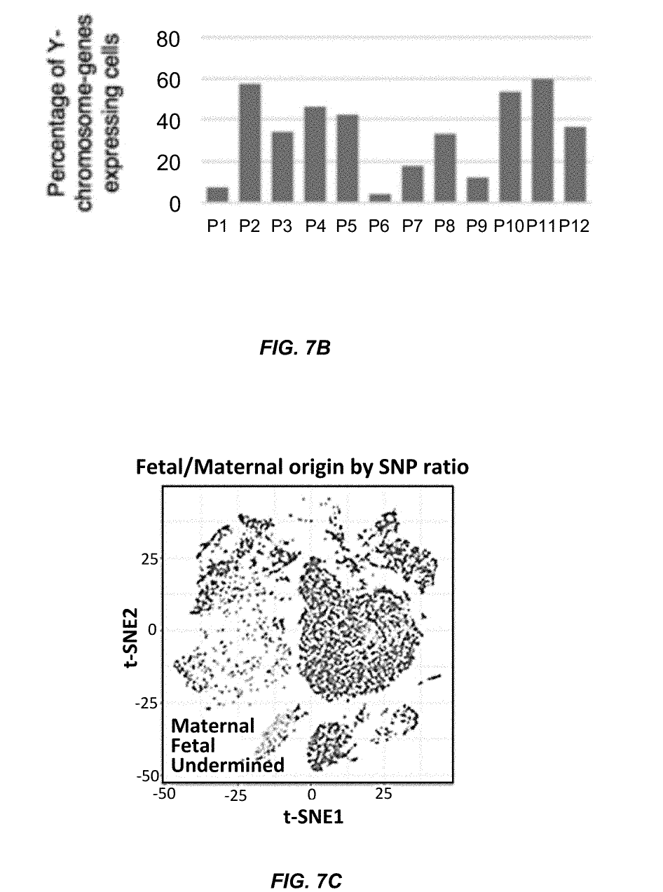

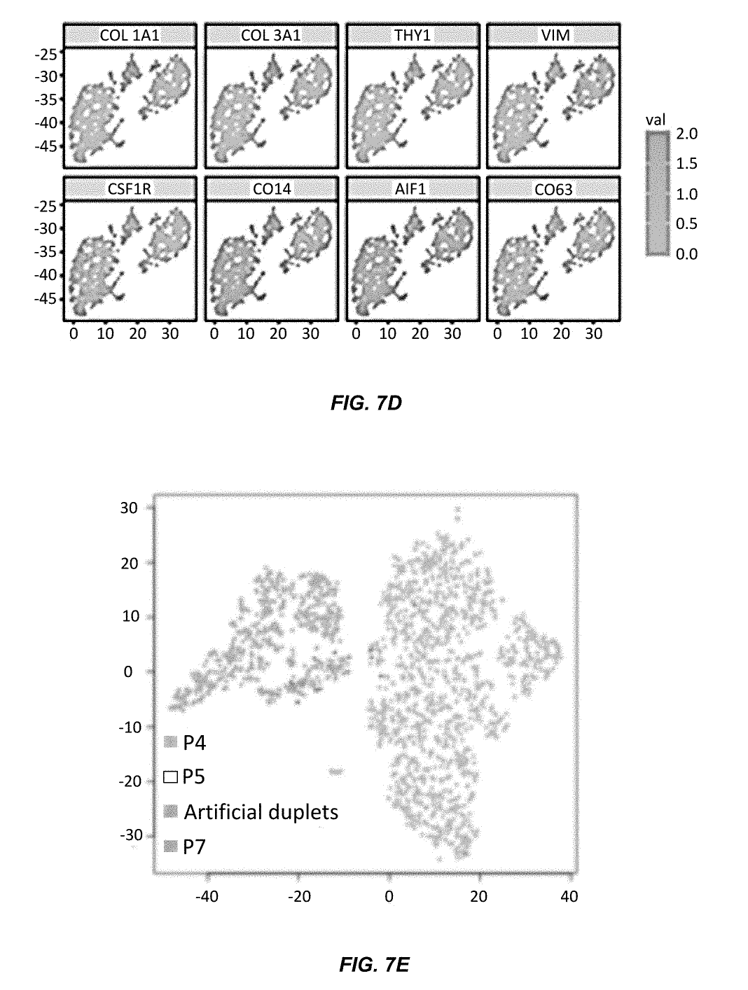

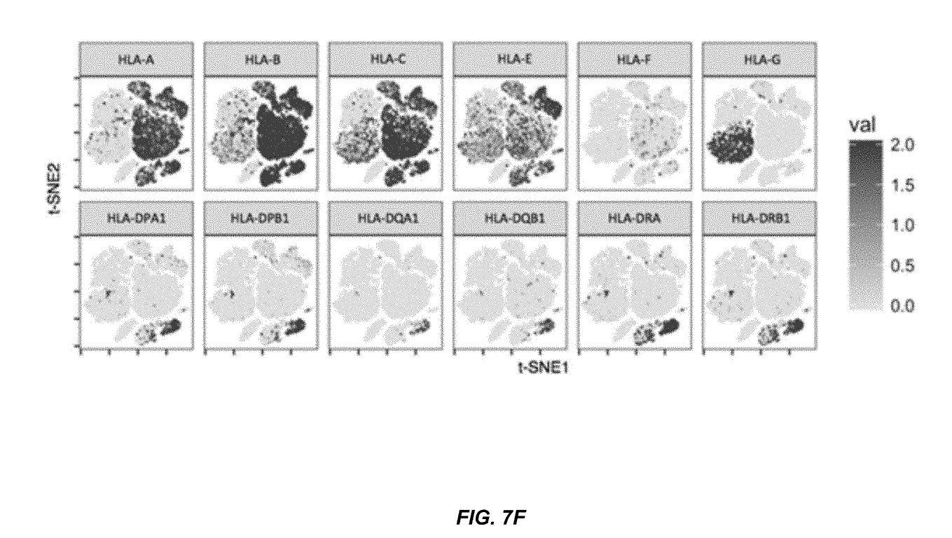

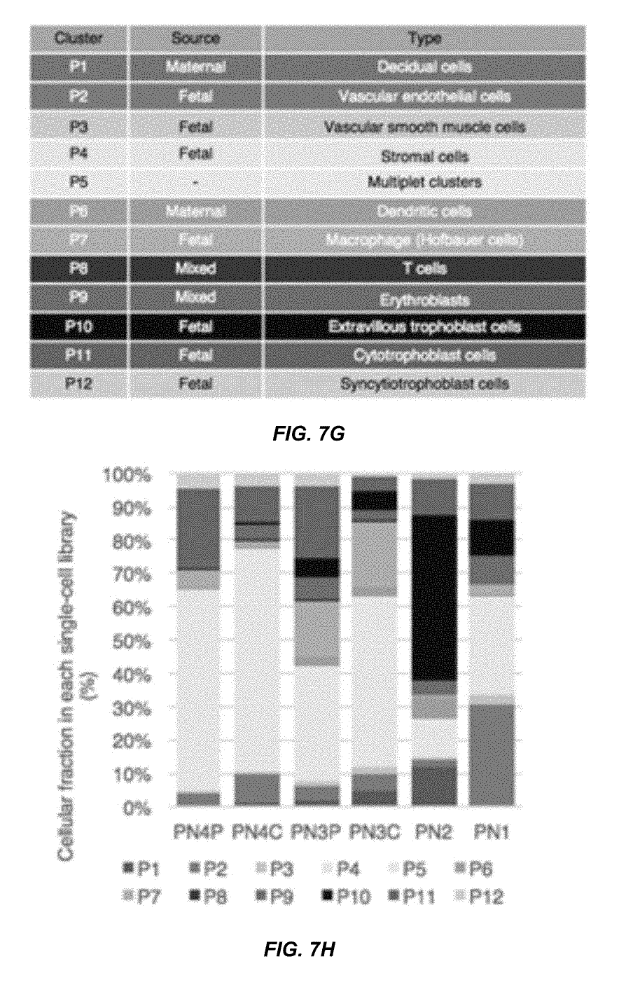

FIG. 7A-H show the dissection of the cellular heterogeneity and annotation of cellular identity in the human placenta. FIG. 7A shows a percentage column chart comparing the fraction of maternal or fetal origin in each cellular subgroup. FIG. 7B shows a column chart comparing the percentage of cells expressing Y-chromosome encoded genes in each cellular subgroup. FIG. 7C shows a biaxial scatter plot showing the distribution of cells of predicted fetal/maternal origin in the original t-SNE clustering distribution as in FIG. 5. Data from PN2 libraries have not been plotted as no genotyping information was available for fetomaternal origin prediction. FIG. 7D shows the expression pattern of stromal (COL1A1, COL3A1, THY1 and VIM) and myeloid (CSF1R, CD14, AIF1 and CD53) markers in P5-7 subgroups. FIG. 7E is t-SNE analysis showing clustering of P5 cells with artificial P4/P7 duplets generated in silico, suggesting that P5 cells are likely multiplets. FIG. 7F is biaxial scatter plots showing the expression pattern of genes encoding for human leukocyte antigens among different subgroups of placental cells. FIG. 7G is a table summarizing the annotated nature of each cellular subgroup. FIG. 7H shows cellular subgroup composition heterogeneity in different single-cell transcriptomic datasets. PN3P/PN3C and PN4P/PN4C represents paired biopsies taken proximal to the umbilical cord insertion sites (PN3C/PN4C) and distal at the periphery of the placental disc (PN3P/PN4P).

Our analysis showed that all clusters, except P1, P6, P8, and P9, are of predominant fetal origin (FIG. 7A,C). P1 transcriptionally corresponds to maternal decidual cells, with strong expression of DKK1, IGFBP1, and PRL, which are known decidual marker genes (FIG. 6). The identity is consistent with the fetomaternal origin we deduced by fetomaternal SNP ratio analysis, which classifies P1 as completely maternal. P6 expressed dendritic markers CD14, CD52, CD83, CD4 and CD86, likely representing maternal uterine dendritic cells (FIG. 6). Meanwhile, P8 expressed high levels of T lymphocyte markers, e.g. CD3G and GZMA. The fetomaternal SNP ratio analysis suggested that P8 is a mixture of both fetal and maternal lymphocytes (FIG. 7A-C). Similarly, the homogenous expression of adult and fetal hemoglobin genes such as HBA1, HBB and HBG1, and the gene encoding the heme biosynthetic enzyme ALAS2 in P9 suggested that they are composed of erythrocytic cells from fetal cord and maternal source. Determining that certain regions are preferentially expressed with certain cells more than other cells is similar to block 212 of FIG. 2.

The rest of the fetal subgroups (P2-5, 7, 10-12) can be broadly classified into four groups, i.e. vascular (P2-3), stromal (P4), macrophagic (P5, P7) and trophoblastic (P11-13) cells. P2 cells commonly expressed strong vascular endothelial markers, e.g. CD34, PLVAP and ICAM. A few cells of maternal origin can also be found in the P2 cluster (FIG. 7C). P3 cells showed features of vascular smooth muscle cells, with expression of MYH11 and CNN1. The large cluster of P4 cells expressed mRNAs of the extracellular matrix proteins ECM1 and fibromodulin (FMOD), both of which are markers of villous stromal cells. Similar to maternal P6 cells, fetal P5 and P7 clusters also highly expressed activated monocytic/macrophagic genes CD14, CSF1R (encoding CD115), CD53 and AIF1. Nonetheless, fetal P5 and P7 subgroups showed additional expression of CD163 and CD209, both being markers of placental resident macrophages (Hofbauer cells) (FIG. 7D). Comparing to P7 cells, the P5 subgroups also showed prevalent expression of fibroblastic and mesenchymal genes shared with P4 stromal cells, such as THY 1 (encoding CD90), collagen genes (COL3A1, COL1A1) and VIM (FIG. 7D). These results raised the possibility that the P5 subgroup may be composed of duplets of P4 and P7 cells during single-cell encapsulation. To corroborate this hypothesis, we performed in silico duplet simulation analysis (FIG. 7E) and our result indicated that P5 cells closely resembled the simulated data and hence likely represented duplets.

The trophoblastic clusters (P10-12) can be divided into three subgroups, i.e. extravillous trophoblasts (P10: EVTBs), villous cytotrophoblasts (P11: VCTBs) and syncytiotrophoblasts (P12: SCTBs), based on the expression of trophoblast subtype-specific genes, PAPPA2, PARP1 and CGA, respectively (FIG. 6). Genes involved in the production of important gestational hormones, including CYP19A1 (encoding aromatase for estrogen synthesis), CGA (human chorionic gonadotropin) and GH2 (human placental growth hormone), are all specifically expressed in SCTBs (P12). It is known that placental EVTBs expressed non-classical form of human leukocyte antigens (HLAs), such as HLA-G, to promote maternal immunotolerance of the fetus with uterine NK cells (27-29). Indeed, we detected strong expression of HLA-G in the EVTBs (P10) subgroup with associated expression of HLA-C and HLA-E (FIG. 7F). Expression of HLA genes in VCTBs and SCTBs were generally scarce, whereas classical HLA-A is specifically expressed in non-trophoblast cells (P1-9). Expression of genes encoding the HLA class II molecules, such as HLA-DP, HLA-DQ and HLA-DR were concentrated in P6 and P7, which is consistent with their antigen presenting function in the maternal dendritic cells and fetal macrophages. Identification of clusters as with particular cell types may not be required before identifying genes with preferential expression.

Previous bulk tissue transcriptomic profiling has shown significant spatial heterogeneity between biopsies taken from different sites of the placenta (30). Comparison of the compositional heterogeneity of different libraries in our dataset also reflected such variations. We included two paired biopsies of the placental parenchyma at sites proximal (PN3C & PN4C) and distal (PN3P & PN4P) to the umbilical cord insertion from two different individuals. (FIG. 4). We found that P1 decidual cells were significantly underrepresented in the PN1 library compared to others. Instead, the P2 fetal endothelial cells fraction was significant higher in PN1 than other libraries, suggesting high contribution from the umbilical vasculature on the fetal surface of the placenta in the PN1 biopsy. In contrast, the PN2 library contained the highest fraction of P1 decidual cells, P6 maternal uterine dendritic cells and P10 EVTBs. The PN2 library likely captured more cells at the deeper fetomaternal interface. Cellular compositions of biopsies obtained from paired proximal and distal middle sections were more comparable, with only significant reduction in decidual cells and increased in EVTBs at the distal site, yet the inter-individual variation remained high (FIG. 7H). These findings highlighted the cellular heterogeneity in the placenta and the necessities of a single-cell analytical approach.

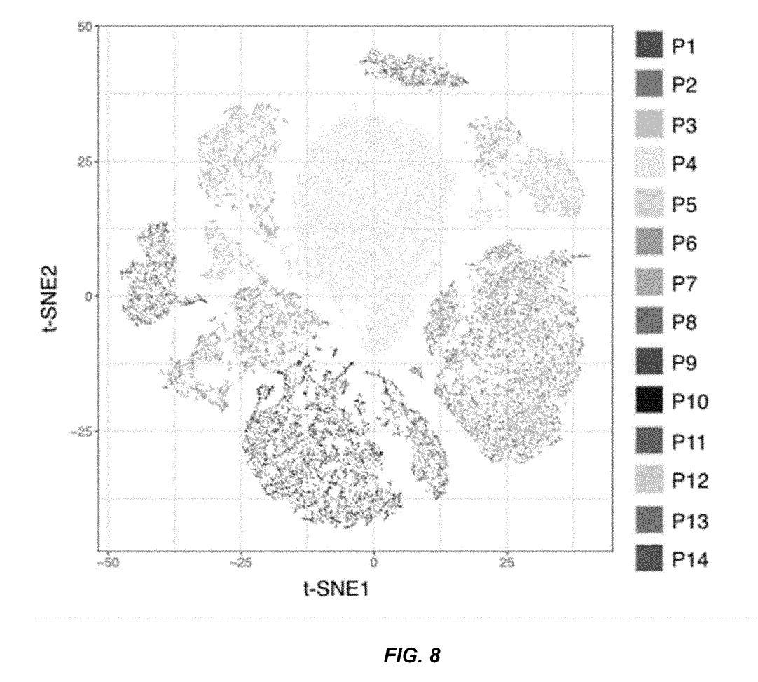

Identification of cell type-specific markers that can be used in plasma RNA analysis may use additional filtering, as it is known that the pool of plasma RNAs is contributed by multiple organ sources, in particularly hematopoietic sources (2, 6). Liver-specific RNA, ALB, is also readily detectable in the plasma (15). To improve cell type specificity, we analyzed the placental dataset with single-cell transcriptomic data of peripheral blood mononucleated cells of healthy donors from public dataset (14) (FIG. 8).

For our data, placental single-cell RNA results and PBMC single-cell RNA sequencing results are obtained separately. We in-silico merged placental single-cell RNA results and PBMC single-cell RNA sequencing results first, then computationally removed the batch biases and performed the clustering analysis. After that, we identified preferentially expressed genes (genomic regions) present in a particular cluster. Such a cluster can be placental cells or PBMC cells or a mixture of placental and PBMC cells. In another embodiment, the experiments for cells derived from different tissues or organs could also be done at the same time and use the barcoding technologies to trace the sample of origins.

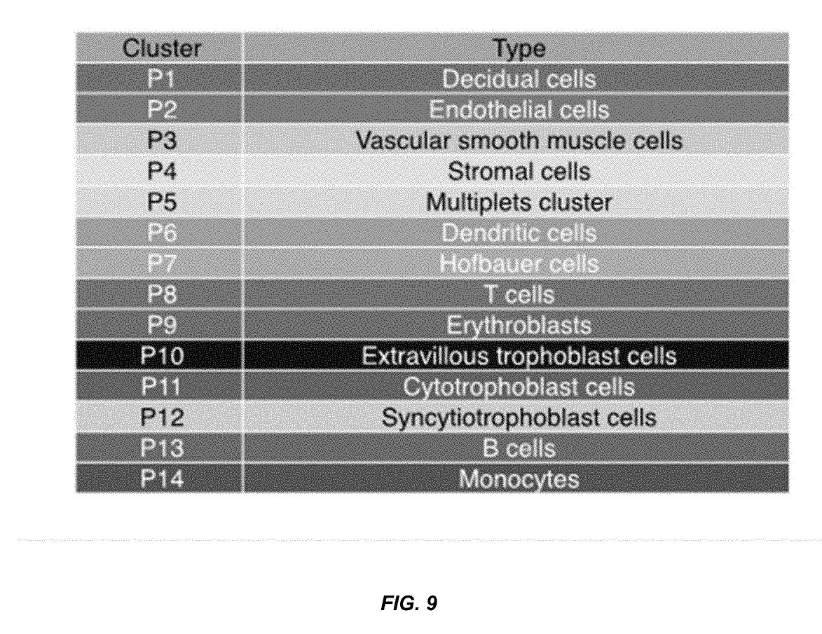

FIG. 8 shows computational single-cell transcriptomic clustering pattern of placental cells and public peripheral blood mono-nucleated blood cells by t-SNE visualization. Each dot in the plot represents the transcriptomic data from a single cell, the proximity of each dot is related to similarities in RNA expression profiles. The clusters are further colored and grouped into subgroups (P1-14) based on spatial proximity and expression pattern of known cell type-specific marker expression. The coloring of the groups corresponds to that of FIG. 5. Based on expression regions and spatial proximity in computational clustering analysis, the clusters correspond to the types shown in FIG. 9

We reasoned that for a gene to be cell type-specific: 1) It should be expressed in the cells of the testing cell type at sufficient high levels and 2) It should not be expressed in other non-testing cells in significant levels, i.e. requiring a minimum expression threshold in the testing cells and maximum expression threshold in the non-testing cells. 3) The magnitude of difference in expression should be meaningfully large, which can be quantified by a minimal threshold value, which can be the absolute difference of expression quantified by certain unit or a mathematically transformed parameters, e.g. relative fold change, log-transformed fold change, standard deviations or normalized standard deviations Z score. In situation where single-cell RNA transcriptomic profiles of a certain tissue in the comparative group is not available, comparisons of whole-tissue RNA profiles can further ensure tissue specificity of the cell type-specific genes, giving that the genes of interest should not show higher expression in other tissues than the tissues of the testing cell type.

B. Noninvasive Elucidation of Placental Cellular Dynamics During Pregnancy

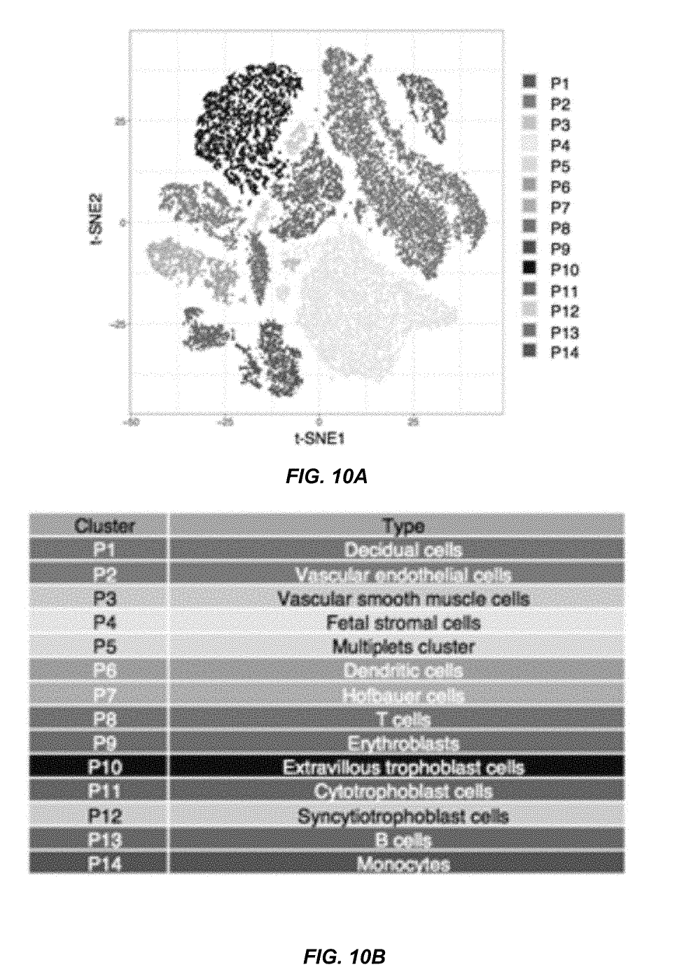

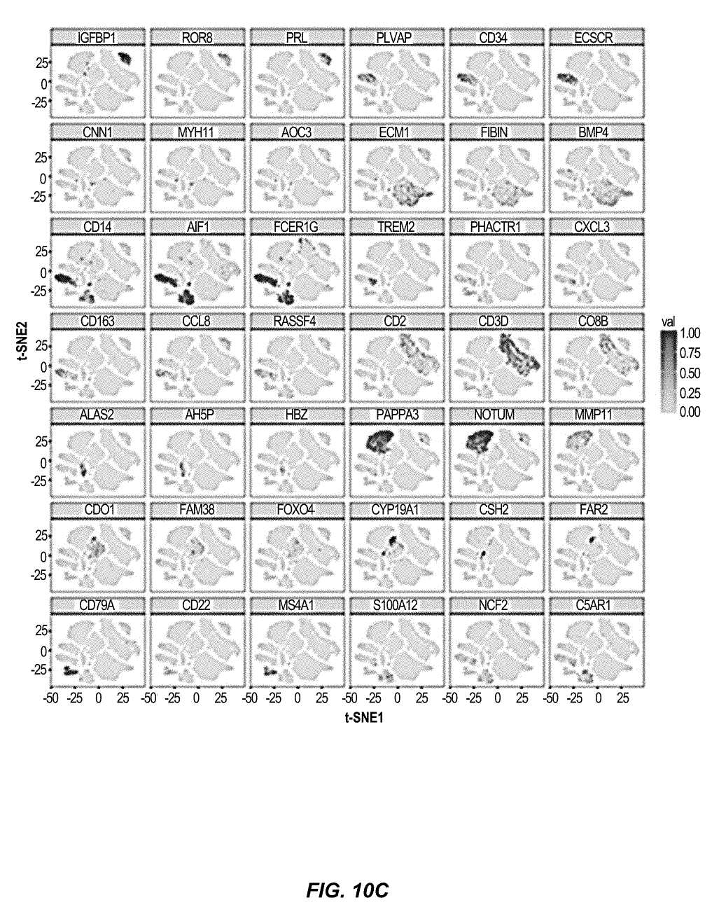

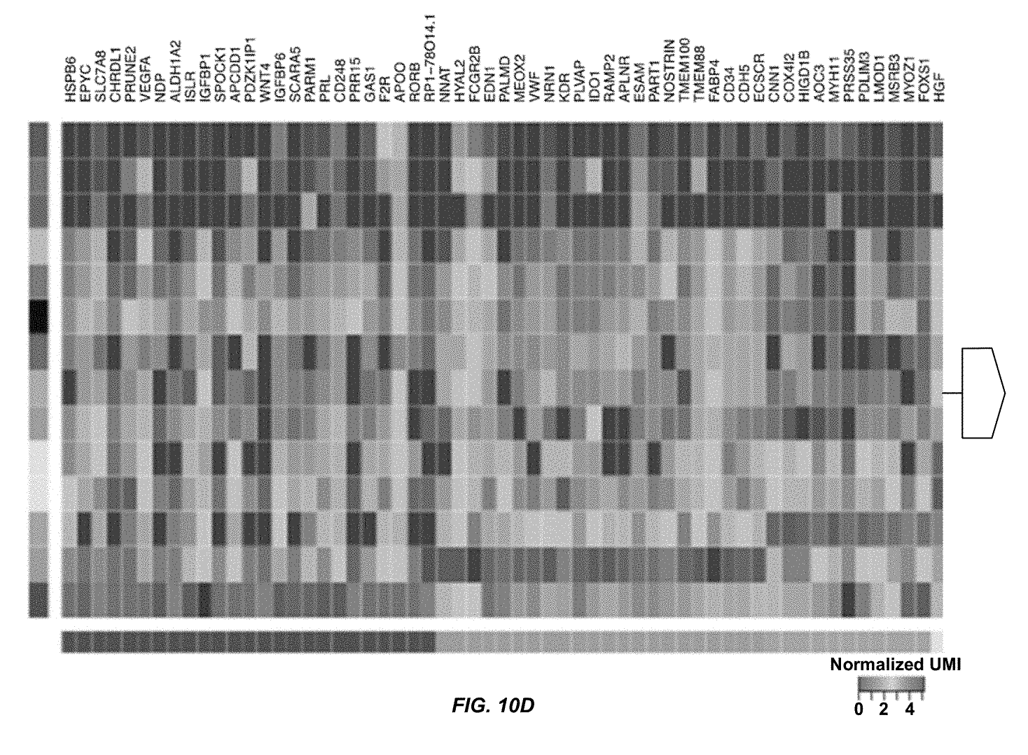

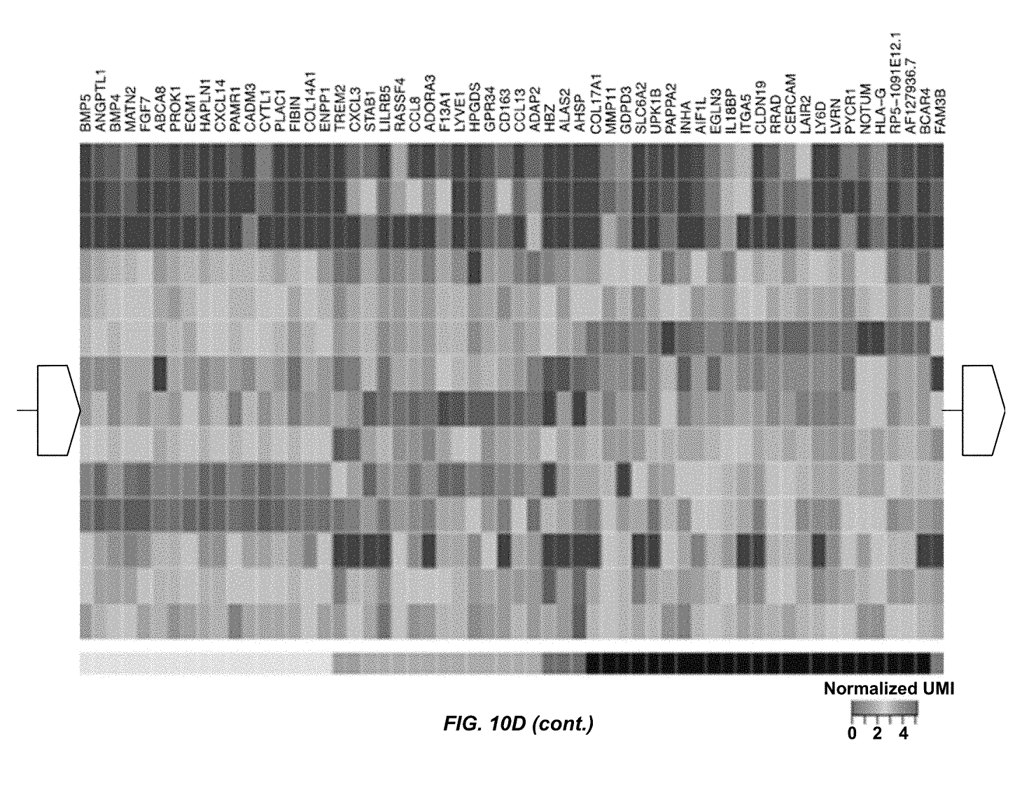

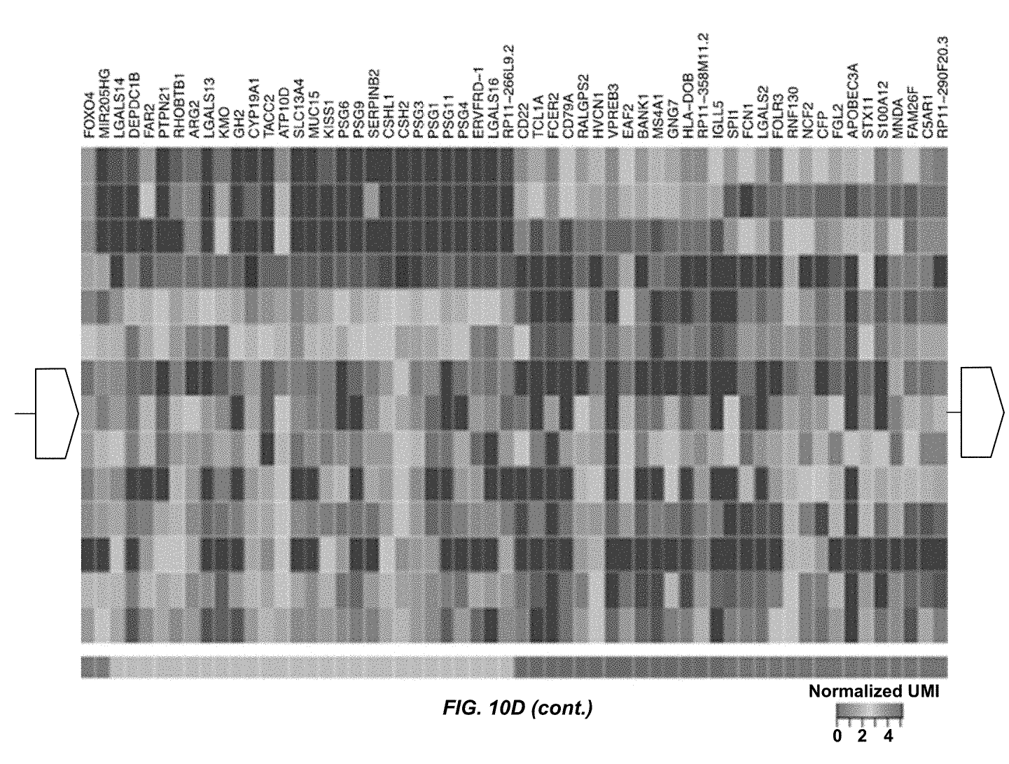

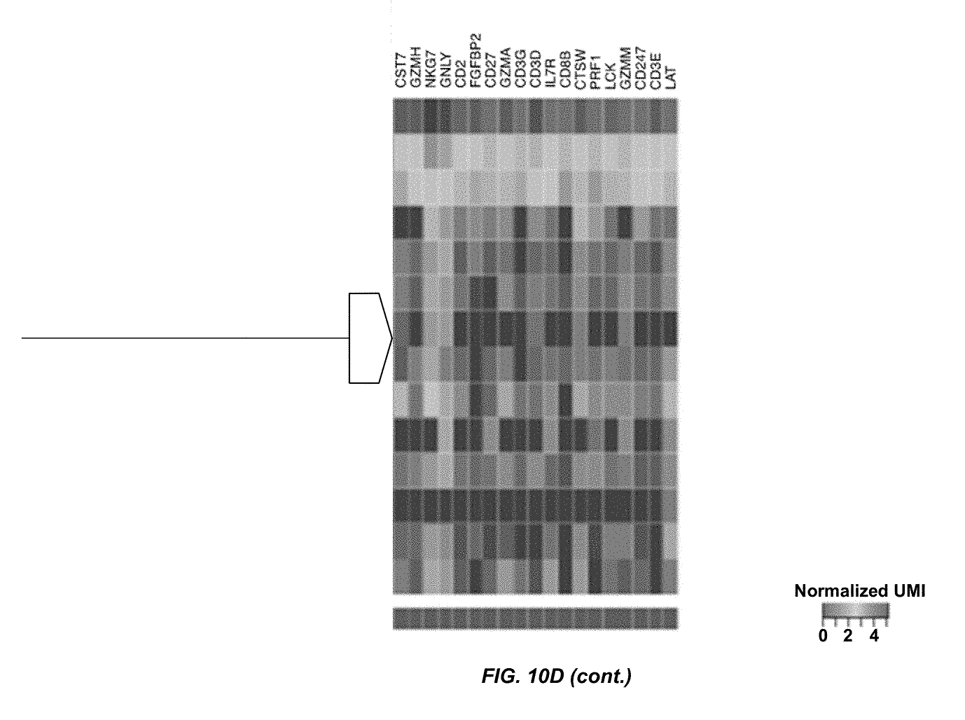

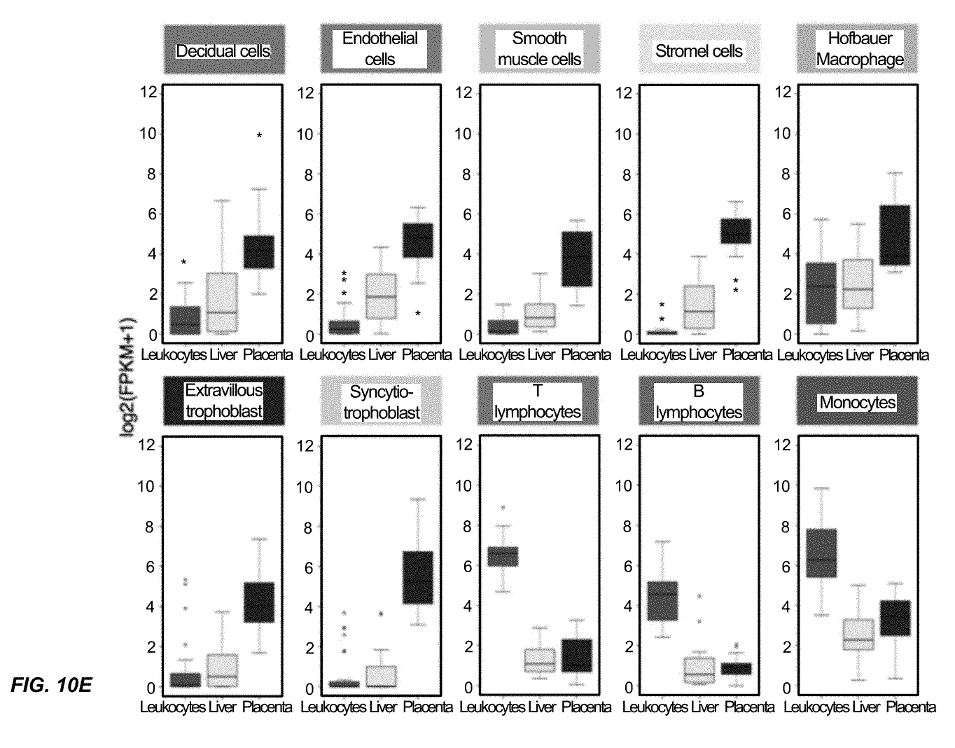

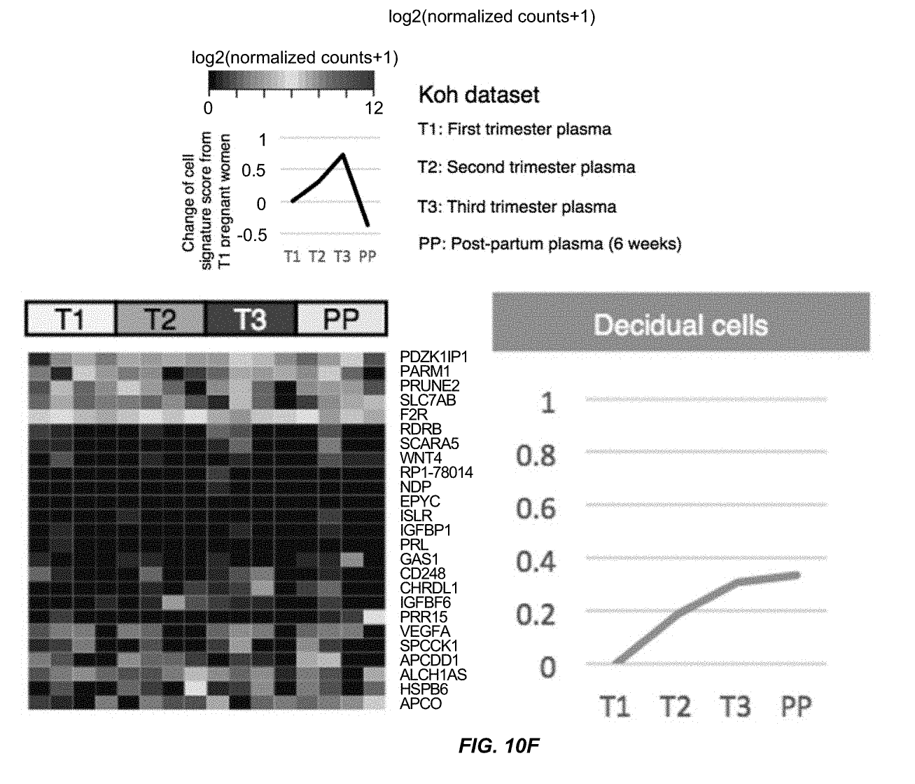

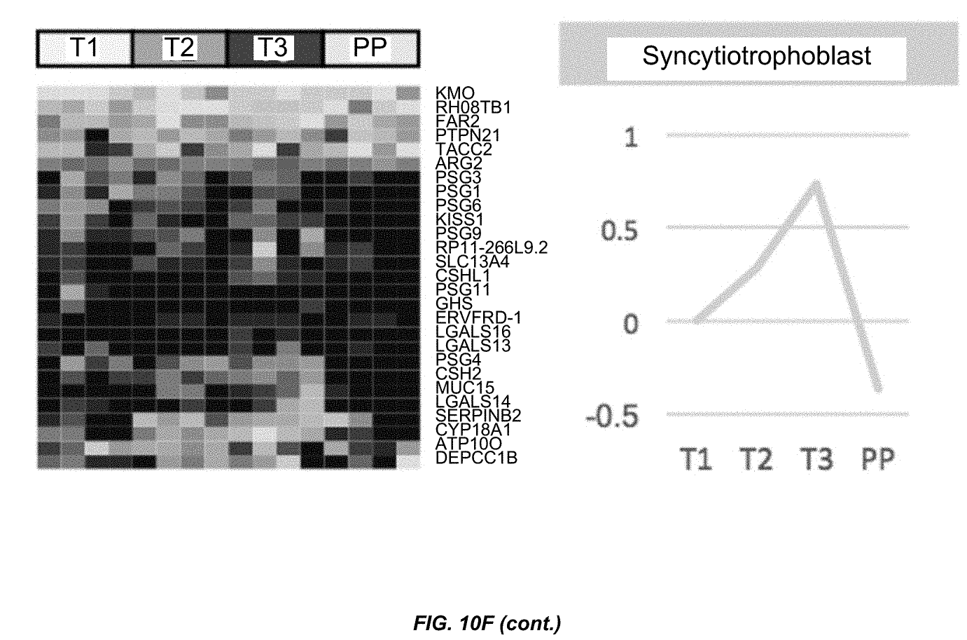

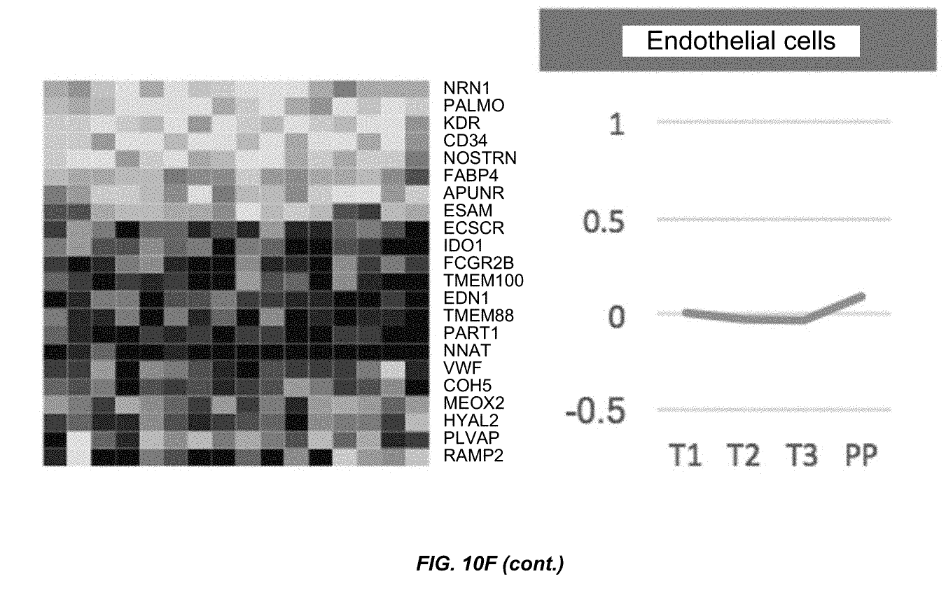

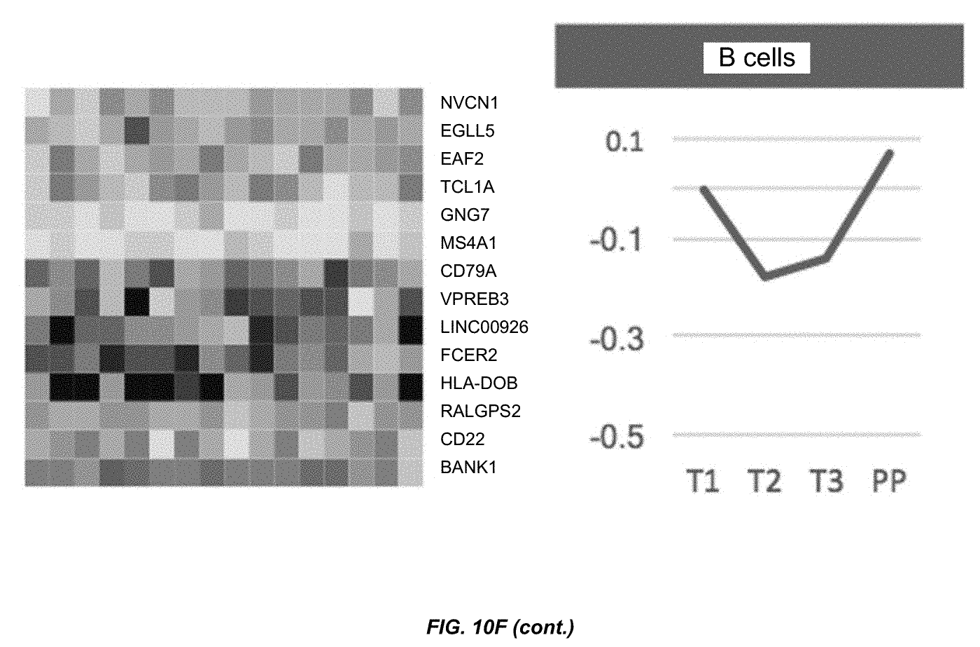

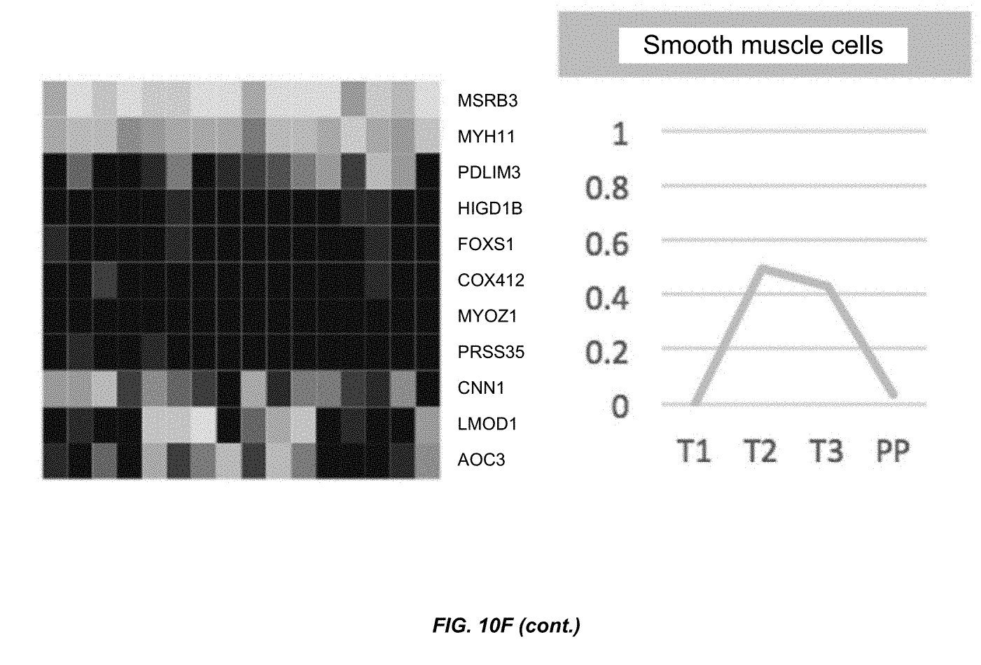

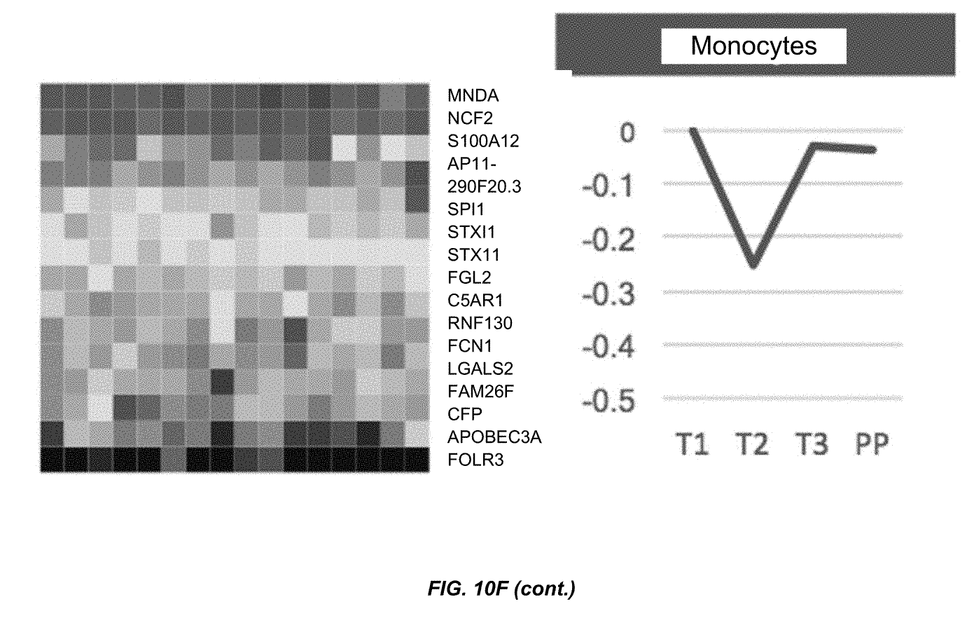

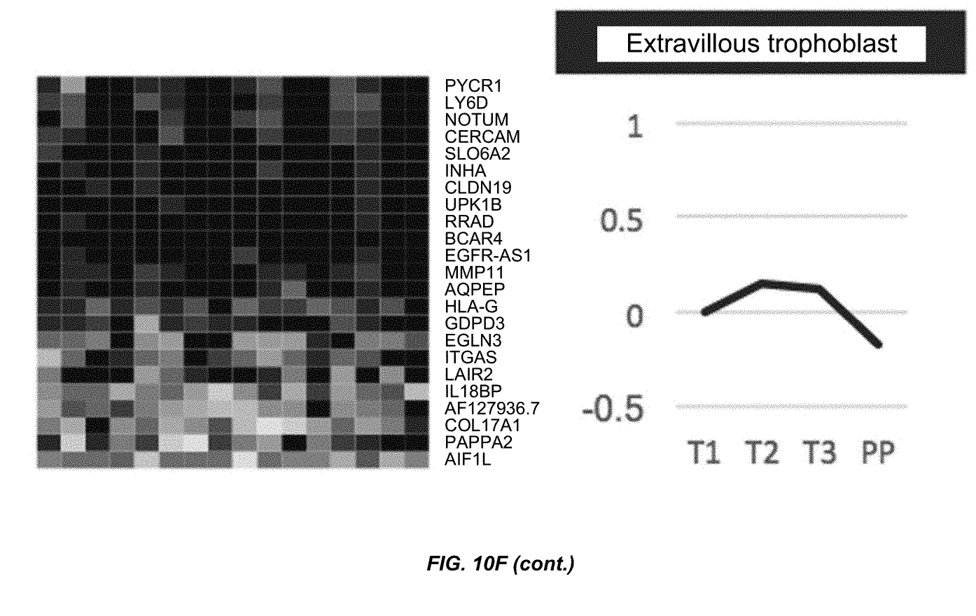

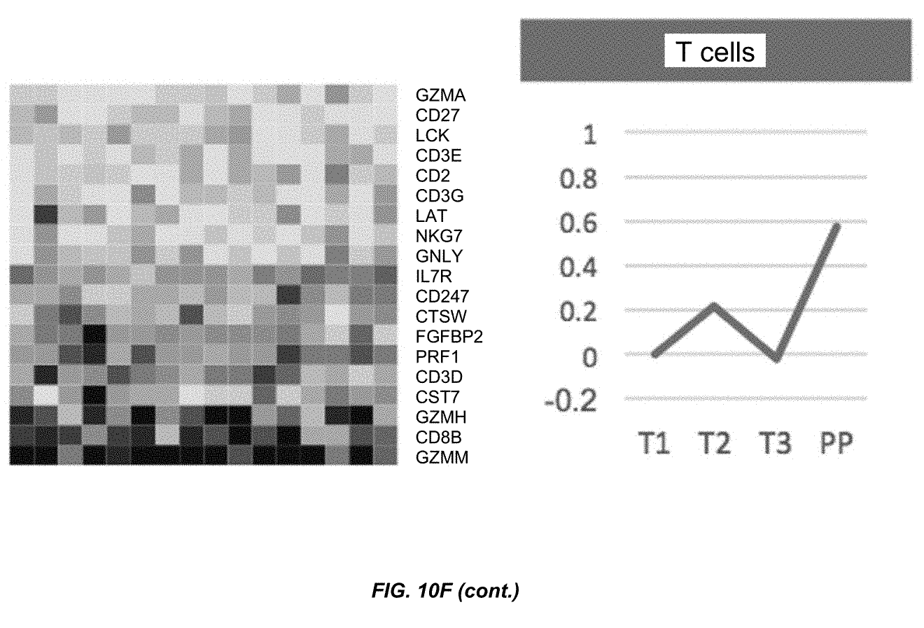

Previous maternal plasma transcriptomic profiling studies showed that certain placenta-specific transcripts and the overall fractional placental contribution increase with gestation ages (21, 34). We hypothesized that it may be possible to dissect the dynamic changes of individual placental cellular components in the maternal plasma cell-free RNAs by establishing the cell type-specific gene signatures at the single-placental cell level. We identified cell type-specific signature genes in P1-12 subgroups by z score comparison. However, it is known that placenta-derived cell-free RNA in maternal plasma circulate in mixture with cell-free RNA derived from hematopoietic source. Donor-specific plasma DNA analysis in sex-mismatched bone marrow transplant recipients and tissue-specific DNA methylation analysis in maternal plasma have shown that about 70% and 10% of the circulating DNA in plasma is hematopoietic and hepatic in origin, respectively (16, 22). To further ensure cell-type expression specificity, we filtered the placental signature genes by reanalyzing the public peripheral blood mononucleated cells (PBMC) single-cell transcriptomic profiles and the tissue transcriptome data from the Human lincRNA Catalog Project (26, 35) (FIG. 10A-E).

FIGS. 10A-E show the identification of cell type-specific signature genes sets and noninvasive elucidation of placental cellular dynamic in maternal cell-free RNA. FIG. 10A shows a biaxial t-SNE plot showing the clustering pattern of peripheral blood mononucleated cells (PBMC) and placental cells. The PBMC data were downloaded from Zheng et al (26). Clusters in FIG. 10A were determined using the placenta single-cell RNA sequencing results merged with PBMC single-cell sequencing data and similar techniques as for diagram 140 in FIG. 1. FIG. 10B shows a table summarizing the annotated nature of each cellular subgroups in the placenta/PBMC merged dataset. FIG. 10C shows biaxial scatter plots showing the expression pattern of specific marker genes among different subgroups of placental cells and PBMC.

FIG. 10D is a heat map showing the average expression of cell-type specific signature genes in different PBMC and placental cells clusters. The colors indicated in the leftmost vertical column correspond to the cell cluster coloring in FIG. 10A. The particular rows associated with a color in the vertical column show the genes used to group cells into the clusters of FIG. 10A. The colors indicated on the topmost row correspond to the cell-type specificity of the particular gene. A box with a red color indicates that the particular gene has a relatively high expression level in a particular cluster, suggesting that the gene is strongly associated with the cell type. A box with a blue color indicates a gene has a relatively low expression level in a particular cluster, and the particular gene is weakly associated with the cell type.

FIG. 10E shows box plots comparing the expression levels of different cell-type specific genes in human leukocytes, the liver, and the placenta. Expression levels of each cell type-specific gene in the whole-tissue profile of the placenta, liver, and leukocytes were compared, and only genes exhibiting the highest expression levels in their corresponding tissue of origins, placenta, or leukocytes were selected. We then excluded cell clusters that contained less than 10 differentially expressed genes or cell clusters in which the differentially expressed genes did not show adequate separation between placenta and leukocyte/liver (P-value>0.05). Among the 14 cell clusters in the PBMC-placenta datasets, no specific genes were identified for cluster P5, and only less than five genes passed the filter for cluster P6, P9, and P11. The gene signature set of P7 representing placental Hofbauer macrophage was excluded from additional analysis because of inadequate separation from leukocytes.

FIG. 10F shows cell signature analysis of the maternal plasma RNA profiles of Koh et al. (21). In Koh, data were collected at each of three trimesters of pregnancy and 6-weeks postpartum. Heat maps showing the expression levels of individual cell-type specific genes in different cell signature gene sets in first trimester maternal plasma (T1), second trimester maternal plasma (T2), third trimester maternal plasma (T3) and postpartum maternal plasma (PP) (left column panels). Line plots showing the change of the average cell signature scores of individual cell-type signature gene sets in different stages of pregnancy (right column panels). The signature analysis may parallel blocks 216 and 218 described with FIG. 2.

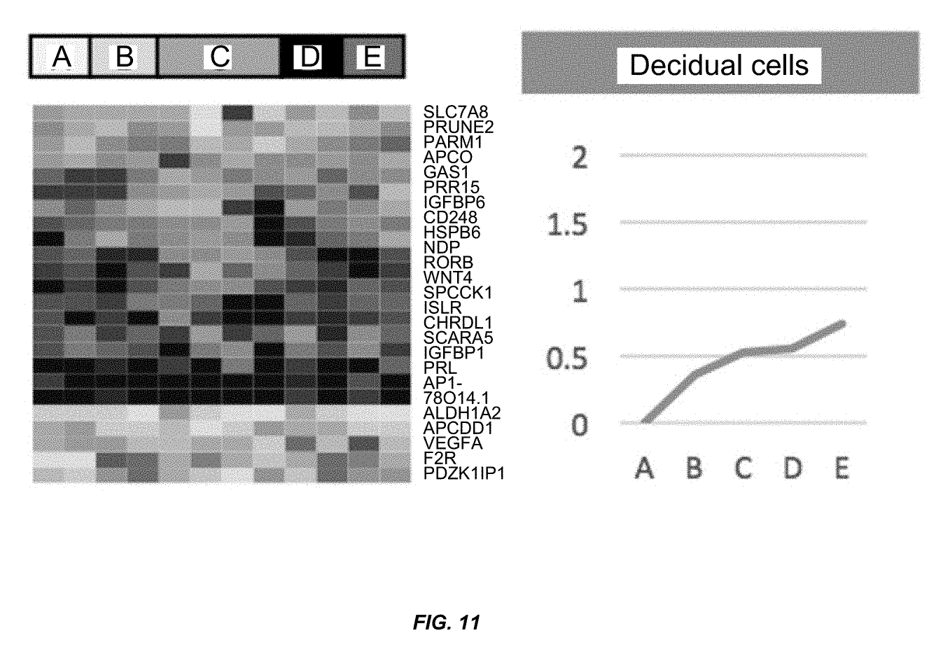

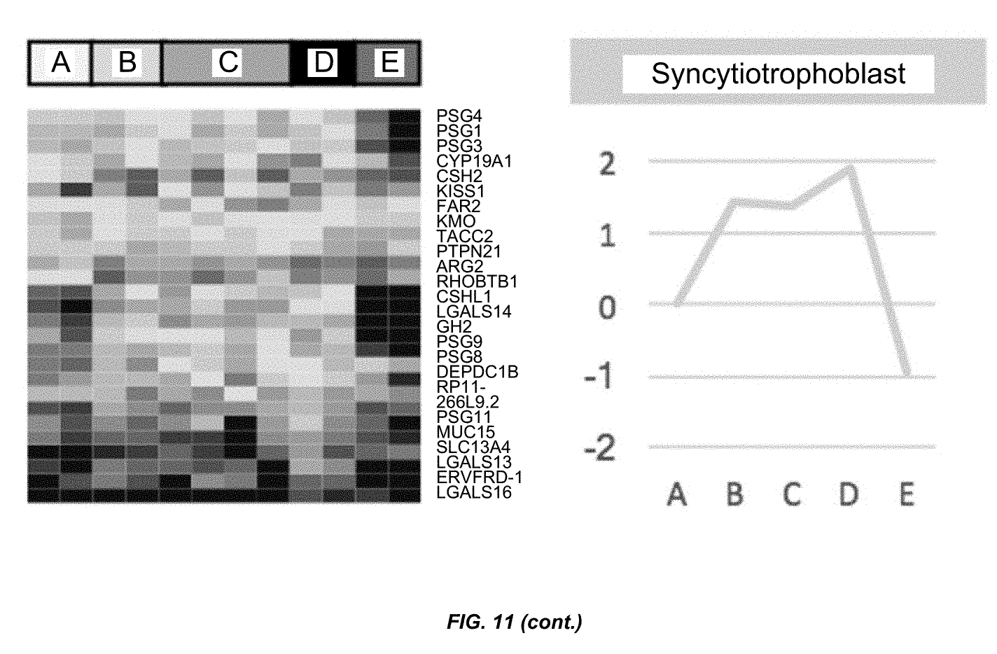

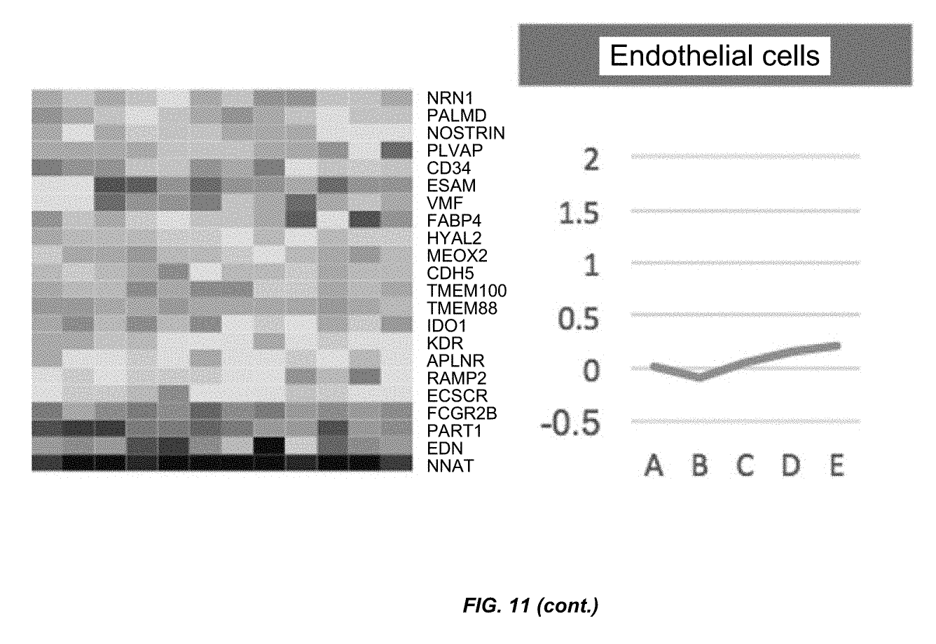

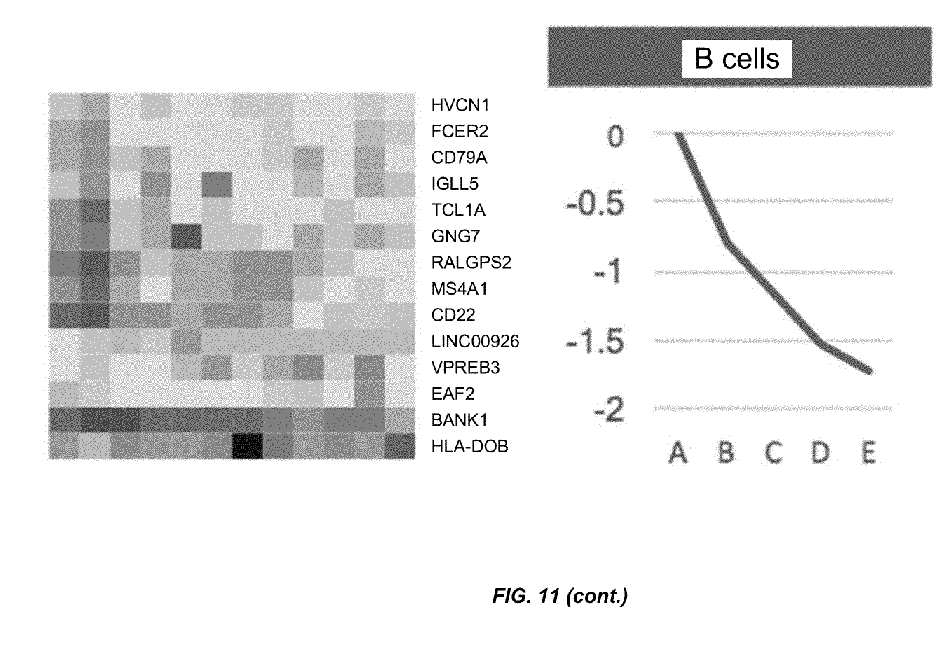

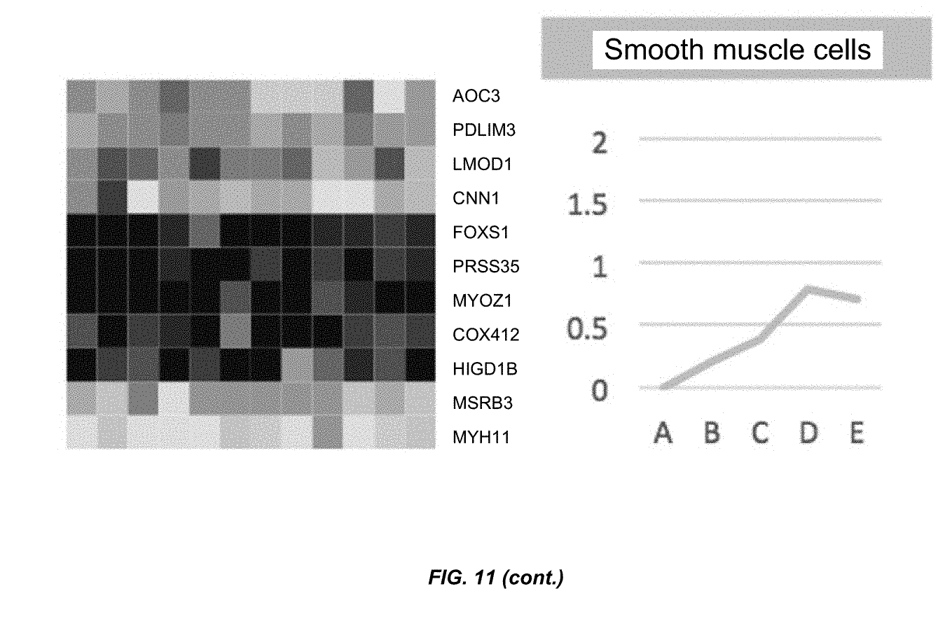

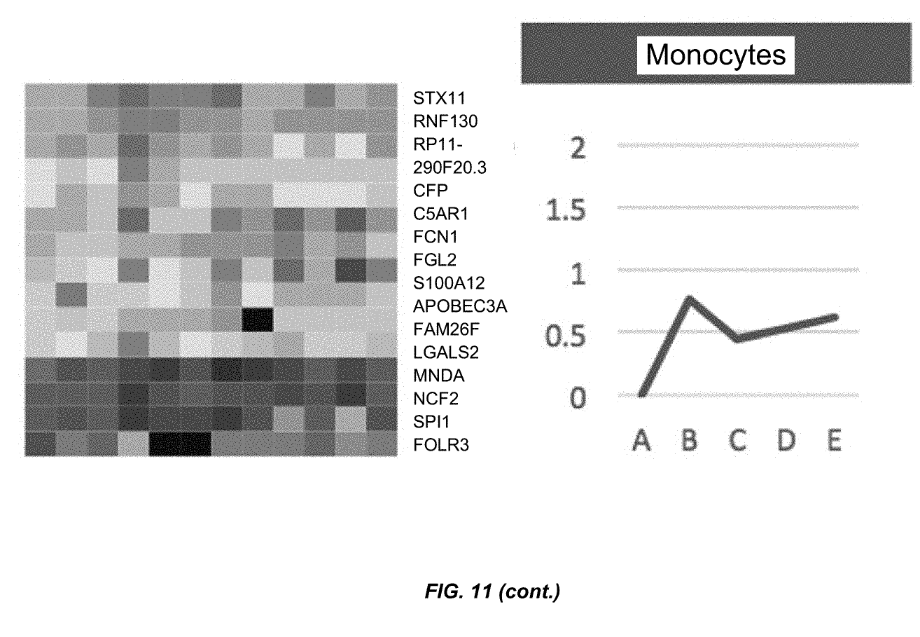

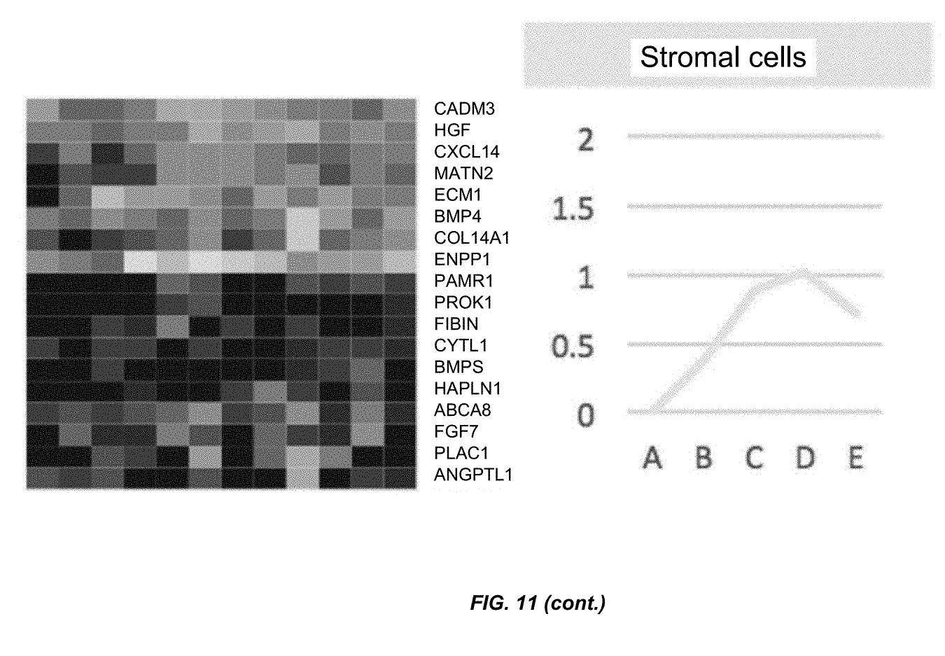

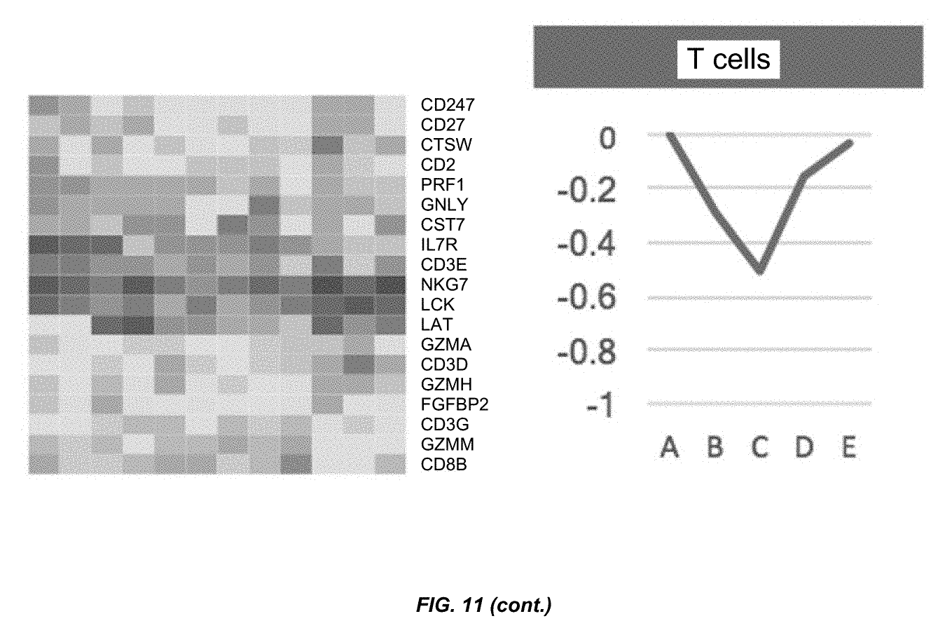

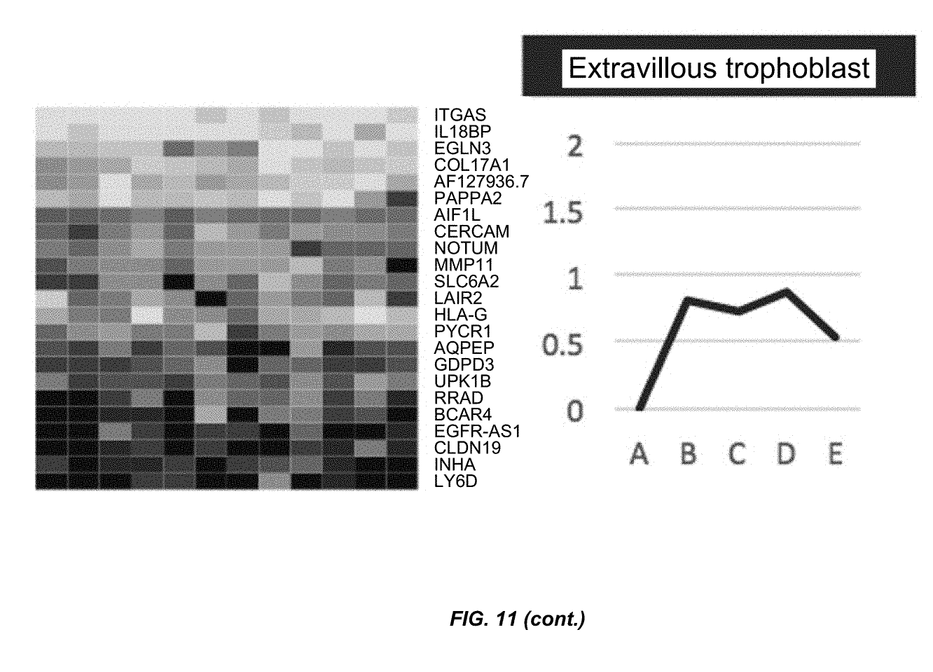

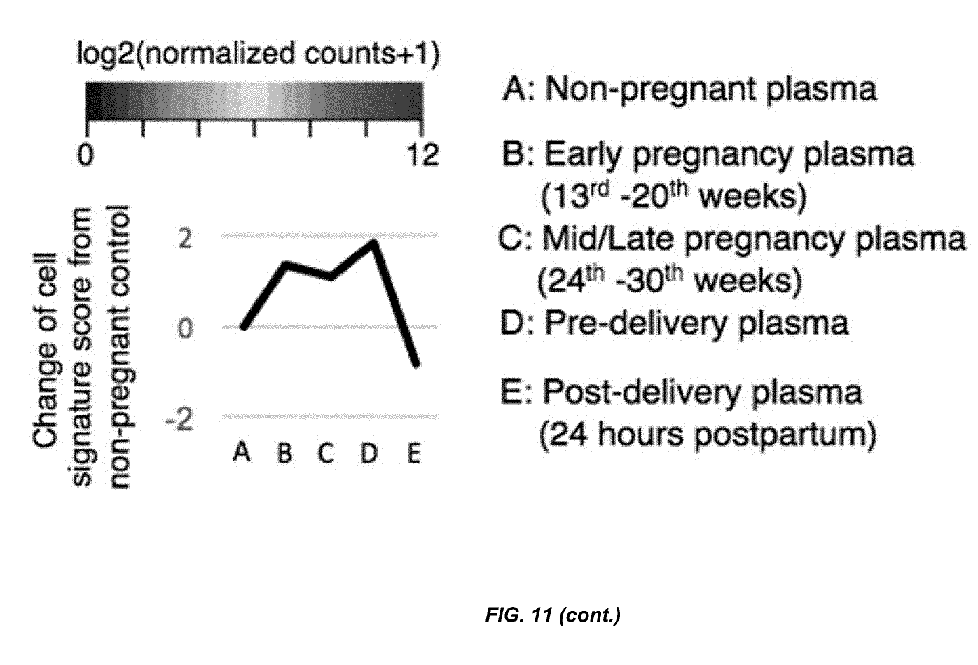

We then studied the longitudinal expression dynamics of the corresponding cell type-specific signature gene sets in the maternal plasma RNA profiles from different stages of pregnancy in a separate dataset by Tsui et al (20). FIG. 11 shows the placental cellular dynamic in maternal plasma RNA profiles during pregnancy. Heat maps in the left column of each panel show the expression levels of individual cell-type specific genes in different cell signature gene sets in non-pregnancy female plasma (group A), early pregnancy maternal plasma (group B), mid/late pregnancy maternal plasma (group C), pre-delivery maternal plasma (group D) and early post-delivery maternal plasma (group E). Line plots in the right column of each panel show the change of the average cell signature scores of individual cell-type signature gene sets in different groups of plasma

With the Tsui dataset, the dynamic patterns of the cell type-specific signature are consistent with the known biological changes during pregnancy. We observed a dramatic upregulation of syncytiotrophoblast (SCTB) signature in the maternal plasma RNA of early pregnancy compared to non-pregnant controls (FIG. 11). The trend peaked at pre-delivery maternal plasma before rapidly dropping to levels of non-pregnant controls 24 hours after delivery. A similar pattern can also be found in the extravillous trophoblast (EVTB), placental stromal cell, and vascular smooth muscle cell signatures. These patterns correspond to the rapid growth of the stromal, SCTB, and EVTB components of the placenta in early pregnancy and clearance after placental delivery. Intriguingly, the signature of decidual cells remained observable in maternal plasma up to 24 hour after delivery. This can be explained by the fact that release of cell-free RNA from residual maternal decidual tissues may continue after placental delivery. In contrast, we found that the signature of B cell continued to drop throughout pregnancy, whereas signature of T cell first dropped and then recovered to non-pregnant levels before delivery. Consistently, previous studies on pregnancy-associated lymphopenia by flow cytometry showed that T and B cells levels decline with the progression of pregnancy (36-38) and peripheral B cell recovery may occur later than T cell (37). Meanwhile, the signature of monocytes shows a more variable pattern, upregulating in early pregnancy, dipped and rebound before delivery, in line with the findings of myeloid immunity activation during pregnancy (36, 39-41). We observed dynamic patterns of cell signature found in the Tsui dataset to be consistent with the Koh dataset (FIG. 10F). These patterns of cell increase and decrease may not be observable with conventional genomic markers that may not be associated with specific cell types.

These findings demonstrated the ability of cell type-specific signatures analysis to dissect individual cellular component dynamics in the maternal plasma RNA profiles. One of the signature scores or a combination of the signature scores could be used in determining the gestational age of future samples.

C. Deciphering Cellular Aberrations in Preeclampsia Placentas from Maternal Plasma Cell-Free RNA

We next demonstrated that signature gene set expression analysis in plasma RNA can detect cellular aberrations in complex diseases. We recruited 10 third trimester normal pregnant controls and 6 women suffering from severe preterm preeclampsia from the Department of Obstetrics and Gynaecology, Prince of Wales Hospital, Hong Kong. We preserved the plasma RNA by mixing TRIzol (Ambion) with plasma in a ratio of 3:1 immediately after plasma isolation and extracted using the RNeasy Mini Kit (Qiagen). We quantified the RNA by NanoDrop ND-2000 Spectrophotometer (Invitrogen) and real-time quantitative PCR targeting GAPDH on a LightCycler 96 System (Roche). We performed cDNA reverse transcription and second strand synthesis by Ovation RNA-seq System V2 (NuGEN). The amplified and purified cDNA was sonicated into 250-bp fragments using a Covaris S2 Ultrasonicator (Covaris) and RNA-seq library construction was constructed by Ovation RNA-seq System V2 (NuGEN). All libraries were quantified by Qubit (Invitrogen) and real-time quantitative PCR on a LightCycler 96 System (Roche), and subsequently sequenced on a NextSeq 500 system (Illumina).

We reasoned that cellular pathology in preeclamptic placentas might affect the release and hence the levels of the cell-type specific RNAs in the maternal plasma. The cellular origin of the pathology can therefore be revealed by comparing the expression levels of different cell type-specific signatures in the maternal plasma of preeclamptic patients with healthy pregnant controls.

We compared the signature gene set expression of multiple cell types between healthy third-trimester pregnancy controls with patients suffering from severe early preeclampsia. We found a specific and significant elevation in the signature gene set of extravillous trophoblast. This is consistent with previous reports that trophoblastic apoptosis is increased in preeclampsia placenta (20-27).

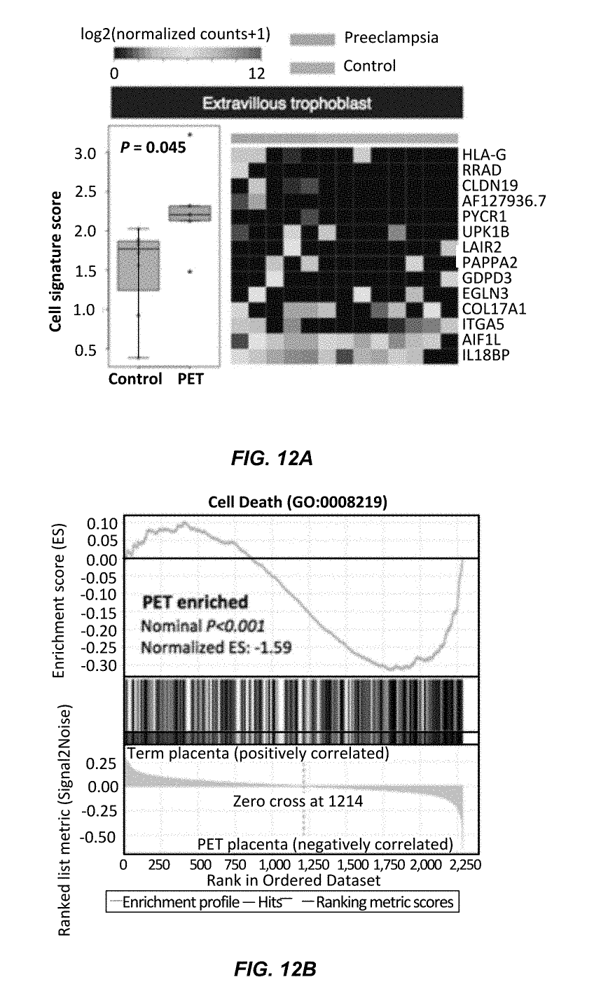

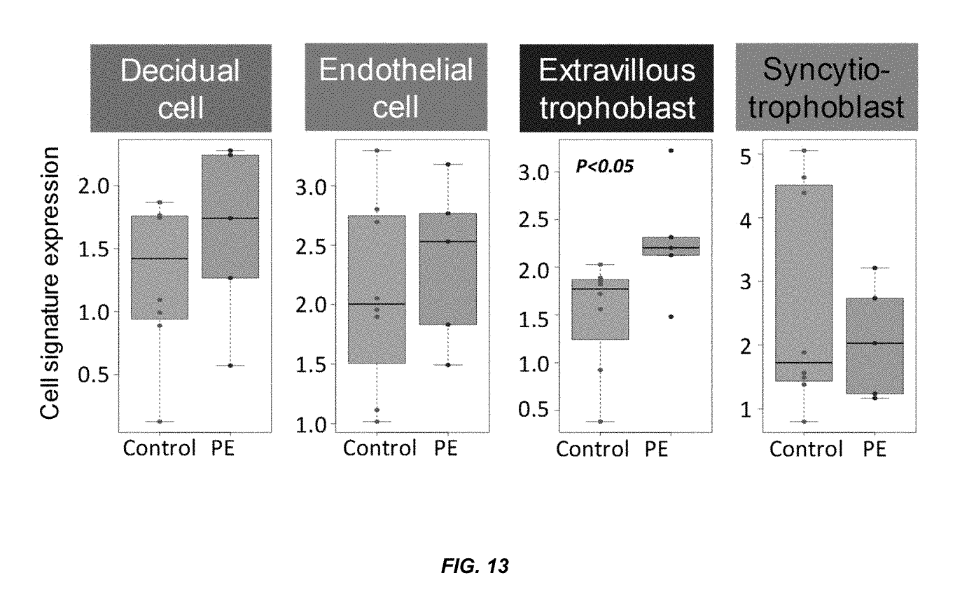

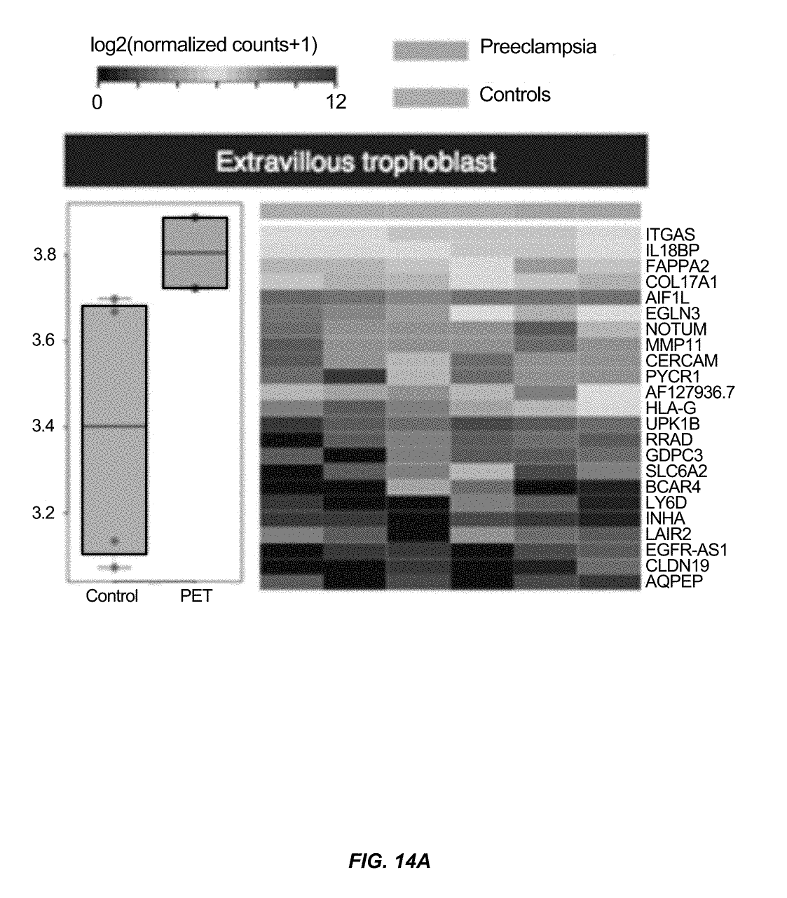

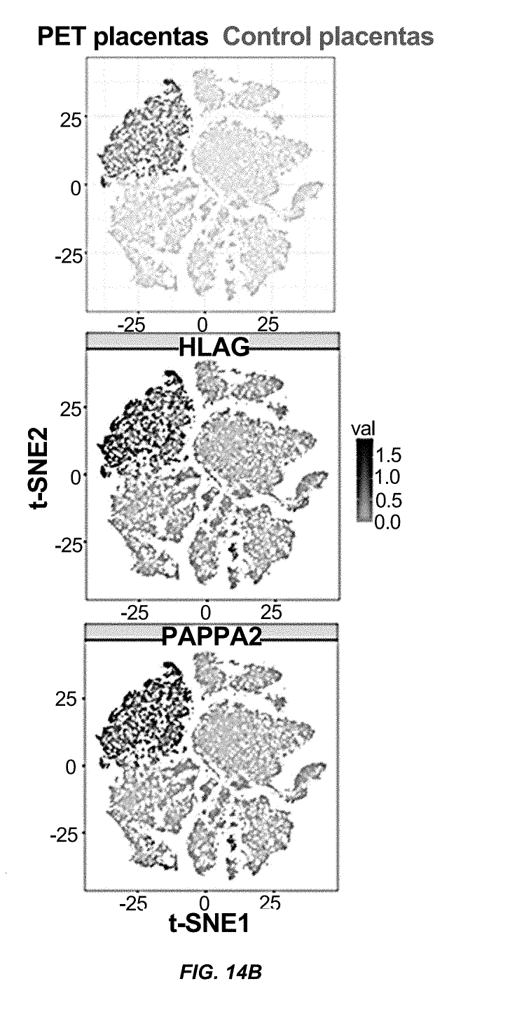

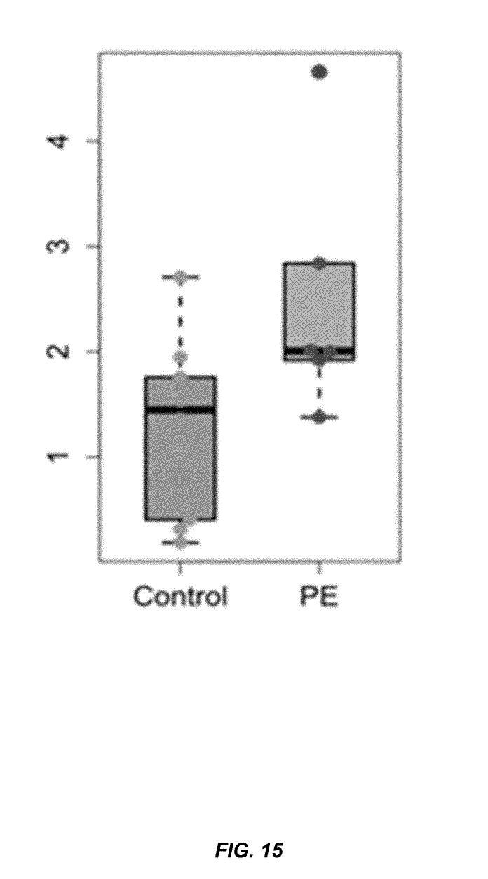

Strikingly, we found that the EVTB signature is consistently upregulated in preeclamptic patients in two separate cohorts assayed with different plasma RNA library preparation chemistries (P=0.045, two-tailed two-sample Wilcoxon signed rank test) (FIG. 12A, FIG. 14A). These results pointed to an increased release of EVTB-derived cell-free RNA into the maternal circulation in preeclampsia. We then validated this finding directly at the tissue level. We characterized the single-cell transcriptome of placental biopsies from four preeclamptic patients and compared the intra-cluster transcriptomic heterogeneity in the HLA-G-expressing EVTB clusters between normal term and preeclamptic placentas to reveal the abnormalities in different biological processes (FIG. 14B). Gene set enrichment analysis also confirmed significant enrichment of cell death-related genes in the preeclampsia EVTB cluster (FIG. 12B). FIG. 13 shows that the signature scores of decidual cells, endothelial cells, and syncytiotrophoblast cells do not have a statistically different signature scores for preeclampsia and control subjects, while the signature score for EVTB is statistically different.

FIG. B10 shows the comparison of cell signature score levels of extravillous trophoblast in maternal plasma samples from third trimester controls and severe early PE patients (p<0.05). Two-sample two-tailed Wilcoxon signed rank test was performed to test for statistical significance. The signature score level for preeclampsia (PE) placentas is significantly different from the controls.

These results suggested that EVTB in preeclampsia placentas have higher levels of cell death. This conclusion is in line with previous reports that trophoblastic apoptosis, in particular for invasive trophoblasts, is increased in preeclampsia (44-51). These offered a mechanistic explanation for the upregulation of the EVTB signatures in the maternal plasma of preeclamptic patients. In short, we demonstrated the ability of plasma cell-free RNA cellular signature analysis as a noninvasive hypothesis-free exploratory tool in revealing hidden cellular pathology of a complex organ source and providing a noninvasive approach for molecular diagnosis of preeclampsia. These results showed that the analytical approach of detecting changes of cell type-specific transcripts discovered through single-cell RNA expression profile analysis in cell-free plasma RNA can be used to detect, differentiate and monitor pathology affecting a complex organ.

D. Discussion

The potential of single-cell transcriptomic analysis on placental biology can be seen in a recent study, where Pavlicev et al profiled 87 microdissected placental cells from the human term placentas and successfully inferred potential inter-cellular communication (54). In this current study, we harnessed the power of microfluidic single-cell transcriptomic technology to establish a large-scale cellular transcriptomic atlas of the human placenta, profiling more than 24,000 non-marker selected cells from normal term and preeclamptic placentas. We annotated the fetomaternal origin of individual cells using both genetic and transcriptional information to provide a comprehensive picture of placental cellular heterogeneity including decidual cells, resident immune cells, vascular and stromal cells.

Finally, we demonstrated the feasibility of integrating single-cell transcriptomic analysis with plasma circulating RNA analysis in dissecting the complex cellular dynamics during normal pregnancy progression and the cellular pathology in preeclampsia placentas noninvasively. Deriving cellular dynamic information using limited known markers is hampered by the high technical variations in detecting the low levels of cell-free RNA in maternal plasma. We overcome this problem by de novo discovery of cell type-specific signature genes from large-scale single-cell transcriptomic profiling, and a gene set analytical basis to harness information of all cell type-specific genes. Comparable cellular dynamic patterns can be observed in two independent maternal plasma RNA datasets (20, 21). The cellular dynamics of trophoblastic and hematopoietic cell types revealed by cell-free RNA cell signature analysis are consistent with some of the known changes in the hematopoietic system and placental during pregnancy. More importantly, this analysis allowed the discovery of differential expression of the EVTB signatures as one of the cellular aberrations in PET patients in a hypothesis-free manner, which reflected pathology at the tissue level. As invasive placental biopsy in healthy pregnant women is infeasible, cell-free RNA cell type-specific signature analysis will be an important molecular tool in exploratory in vivo studies to differentiate cellular pathology in different forms of placental dysfunction and offer clinical diagnostic information. With continuous improvement in the cost-effectiveness of large-scale single-cell transcriptomic technology and the effort of the Human Cellular Atlas Initiative in profiling the cellular transcriptomic heterogeneity of all cellular subtypes in major human organs (26, 56-58), it can be envisioned that the same approach can be extended to other situations such as tumor clonal dynamics dissection in cell-free tumor RNAs and noninvasive exploration of the cellular pathology in other gestational diseases.

In short, our study established a large-scale single-cell transcriptomic atlas of the normal and preeclamptic placentas and showcased the power of integrative analysis of single-cell transcriptomics and plasma cell-free RNA as a novel noninvasive tool for the elucidation of cellular dynamics and aberrations in complex biological systems and molecular diagnosis.

E. Materials and Methods

1. Subjects, Sample Collection and Processing

This study was approved by the institutional ethics committee and informed consent was obtained after the nature and possible consequences of the studies were explained. Healthy or severely preeclamptic pregnant women (FIG. 4) were recruited from the Department of Obstetrics and Gynaecology, Prince of Wales Hospital, Hong Kong with informed consent. We recruited patients with early onset preeclampsia requiring delivery at 24-33⁺⁶ weeks' gestation with blood pressure ≥140/90 mmHg on at least 2 occasions 4 hours apart developing after 20 weeks' gestation with proteinuria of ≥300 mg in 24 hours or ≥30 mg/mmol in protein/creatinine ratio or 2 readings of ≥2+ on dipstick analysis of midstream or catheter urine specimens if no 24-hour collection is available. Only patients with delivery by cesarean section were recruited.

For each case, 20 mL of maternal peripheral blood was collected into EDTA-containing tubes before elective cesarean section. Plasma was isolated by a double centrifugation protocol as previously described (20). For placental parenchymal biopsy, 1 cm³ placental tissue was dissected freshly after delivery from a region 2 cm deep and 5 cm away from the umbilical cord insertion after peeling of the membrane. In some cases, a peripheral site of tissue sampling was also taken from the placental rim (periphery). The dissected tissues were then washed in PBS. Tissues were then subjected to enzymatic digestion using the Umbilical Cord Dissociation Kit (Miltenyi Biotech) according to manufacturer's protocol. Red blood cells were lysed and removed by ACK buffer (Invitrogen). Cell debris was removed by a 100 μm filter (Miltenyi Biotech) and the single cell suspension was washed again three times in PBS (Invitrogen). Successful dissociation was confirmed under a microscope.

2. Plasma and Bulk Tissue RNA Extraction and Library Preparation

Plasma RNA was preserved by mixing TRIzol (Ambion) with plasma in a ratio of 3:1 immediately after plasma isolation. Plasma RNA was then extracted using the RNeasy Mini Kit (Qiagen). All extracted RNA was quantified by NanoDrop ND-2000 Spectrophotometer (Invitrogen) and Real-time quantitative PCR on a LightCycler 96 System (Roche). cDNA reverse transcription and second strand synthesis were done by Ovation RNA-seq System V2 (NuGEN) according to the manufacturer's protocol. Amplified and purified cDNA was sonicated into 250-bp fragments using a Covaris S2 Ultrasonicator (Covaris). RNA-seq library construction was done by Ovation RNA-seq System V2 (NuGEN) according to manufacturer's instructions. All libraries were quantified by Qubit (Invitrogen) and real-time quantitative PCR on a LightCycler 96 System (Roche).

3. Single Cell Encapsulation, in-Droplet RT-PCR and Sequencing Library Preparation

Single cell RNA-seq libraries were generated using the Chromium Single Cell 3′ Reagent Kit (10× Genomics) as described (26). Briefly, single cell suspension without prior selection (cell concentration between 200 to 1000 cells/μl PBS) was mixed with RT-PCR master mix and loaded together with Single Cell 3′ Gel Beads and Partitioning Oil into a Single Cell 3′ Chip (10× Genomics) according to manufacturer's instructions. RNA transcripts from single cells were uniquely barcoded and reverse transcribed within droplets. cDNA molecules were pre-amplified and pooled followed by library construction according to manufacturer's instructions. All libraries were quantified by Qubit and real-time quantitative PCR on a LightCycler 96 System (Roche). The size profiles of the pre-amplified cDNA and sequencing libraries were examined by the Agilent High Sensitivity D5000 and High Sensitivity D1000 ScreenTape Systems (Agilent), respectively.

4. Sequencing, Alignment and Gene Expression Quantification

All single-cell libraries were sequenced with a customized paired-end (PE) with dual indexing (98/14/8/10-bp) format, according to the manufacturer's recommendation. The data were aligned mapped to the human reference genome and quantified as number of unique molecular identifiers (UMIs) using the Cell Ranger Single-Cell Software Suite (version 1.0) as described by Zheng et al (26). In short, samples were demultiplexed based on the 8 bp sample index, 10 bp UMI tags and the 14 bp GemCode barcode. The 98 bp-long read 1 containing the cDNA sequence was aligned using STAR (59) against the hg19 human reference genome. UMI quantification, GemCode and cell barcodes filtering based on error detection by Hamming distance were performed as described by Zheng et al (26).

For alignment of the plasma RNA library, adaptor sequences and low quality bases on the fragment ends (i.e., quality score, <5) were trimmed and reads were aligned to the human reference genome (hg19) using the TopHat (v2.0.4) with the following parameters: transcriptome-mismatches=3; mate-std-dev=50; genome-read-mismatches=3 with the pair-end alignment option as well as the annotated gene model file downloaded from UCSC (http://genome.ucsc.edu/). Gene expression quantification was performed by an in-house script quantifying the number of reads overlapping with exonic regions on genes annotated in the Ensembl GTFs (GRCh37.p13).

All libraries were sequenced on a MiSeq system (Illumina) or a NextSeq 500 system (Illumina) using the Miseq Reagent v3 Kit (Illumina) or NextSeq 500 High Output v2 Kit (Illumina), respectively.

5. Fetal and Maternal Origin Determination

To differentiate the genetic origin of the cell, maternal and fetal genotypes were first determined by the iScan system (Illumina) using buffy coat and placenta tissues, respectively. Genotype information of case M12491 (PN2) was not available due to limitation of biopsy materials. Informative SNPs covered by sequencing reads were then identified, in which a SNP is classified as maternal-specific when it is heterozygous in the mother (AB) and homozygous in the fetus (A/A). Fetal-specific SNPs were classified vice versa. Next, we calculated the allele ratio (R) as fellow:

$$R = \frac{B}{( A + B )}$$

B: Allelic count of the origin-specific SNP B
A: Allelic count of the common SNP A.

Fetal-specific allelic ratio (R_{f}) and maternal-specific allelic ratio (Rₘ) were obtained for each cell. A cell would be annotated as 1) fetal origin, if R_{f}>2) maternal origin, Rₘ>R_{f}; 3) undetermined, if Rₘ=R_{f} or if there are no reads covering any informative SNPs.

6. Duplet Simulation

Gene expression matrix of 1365 P4 cells and 526 P7 cells were first extracted from the PN3C dataset. To emulate 100 duplet data points, the transcriptome of the duplet was modeled as random mixture of 1 P4 cell and 1 P7 cell. The gene expression levels of the artificial duplets were set as the average of the two cells. PCA was then performed. The first 10 factors after PCA analysis were further utilized to carry out the t-SNE clustering. The prcomp and Rtsne package in R were employed during the clustering step for PCA and t-SNE, respectively.

7. Identification of Cell-Specific Genes

Single-cell transcriptomic data of peripheral blood mononucleated cells were retrieved from the public domain of 10× Genomics at https://support.10×genomics.com/single-cell/datasets. The dataset was previously published (26). The PBMC dataset were merged with the placenta dataset and normalized by random read subsampling using the cellrangerRkit version 0.99.0 package. t-SNE clustering was performed with built-in functions in the cellrangerRkit package using the first 10 principal components. Cells clusters were topologically identified in the biaxial t-SNE plots based on known marker gene expression and spatial proximity.

The criteria for cell type-specific gene selection is as follows:
1. Genes with expression z score greater than 3, AND
   Gene expression z scores is calculated as:

$${z}_{g} = \frac{{g}_{A} - {g}_{\overline{A}}}{{s}_{\overline{A}}}$$

z_{g}: z score for gene g
g_{A}: average expression level in cell type A, (log 2-transformed normalized UMI count)
g_{Ā}: average expression level in non-A cells
s_{Ā}: standard deviation of expression in non-A cells.
2. Average gene expression levels (log 2-transformed normalized UMI) in testing cell type greater than threshold (>0.1), AND
3. Average gene expression levels (log 2-transformed normalized UMI) in non-testing cells less than threshold (<0.01) AND
4. The gene expression levels (log-transformed FPKM) in whole tissue profile of liver, placenta and white blood cells from the Human lincRNA Catalog Project (14, 16) showing the highest expression in their source organs, i.e. genes from cell groups annotated as placental cells showing the highest expression in the whole tissue profiles of placenta, comparing to liver and white blood cells; genes from cell groups annotated as white blood cells (P8, P9, P13 and P14 genes) showing the highest expression in the whole tissue profiles of white blood cells, comparing to liver and placenta.

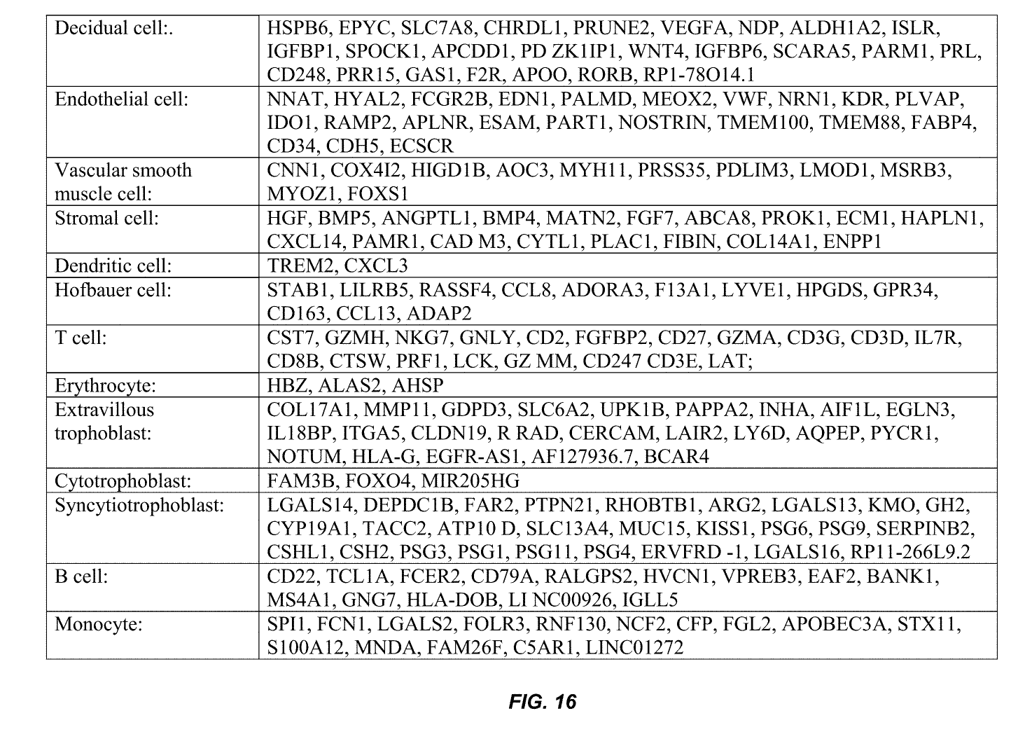

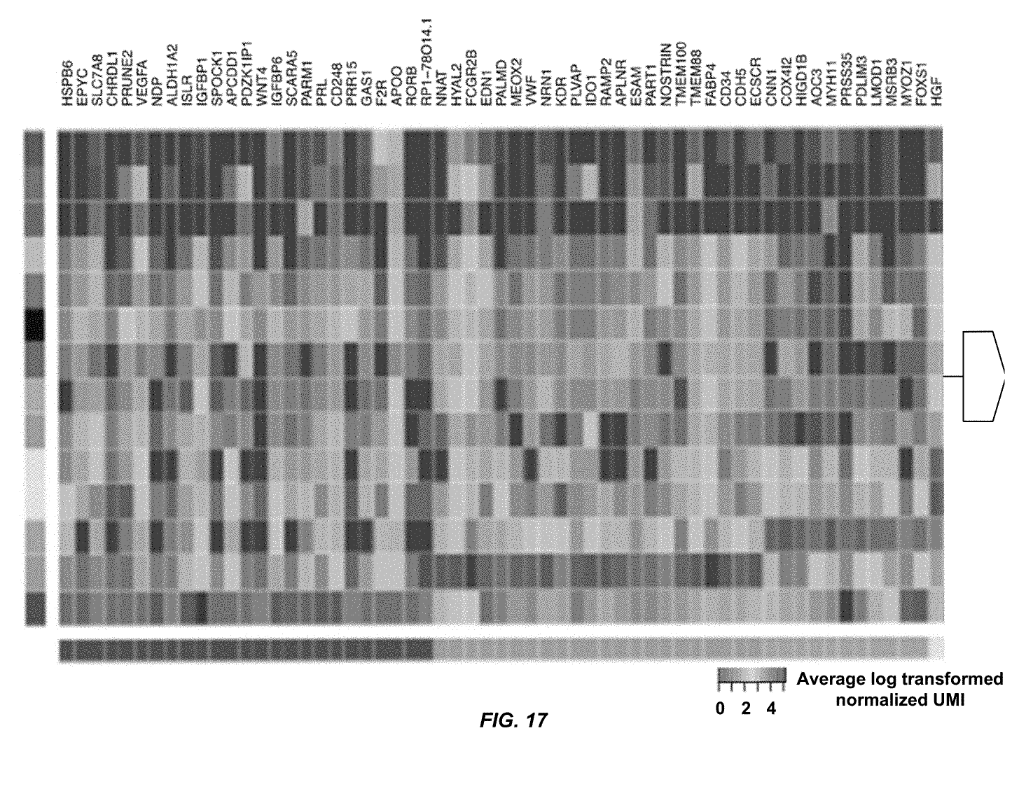

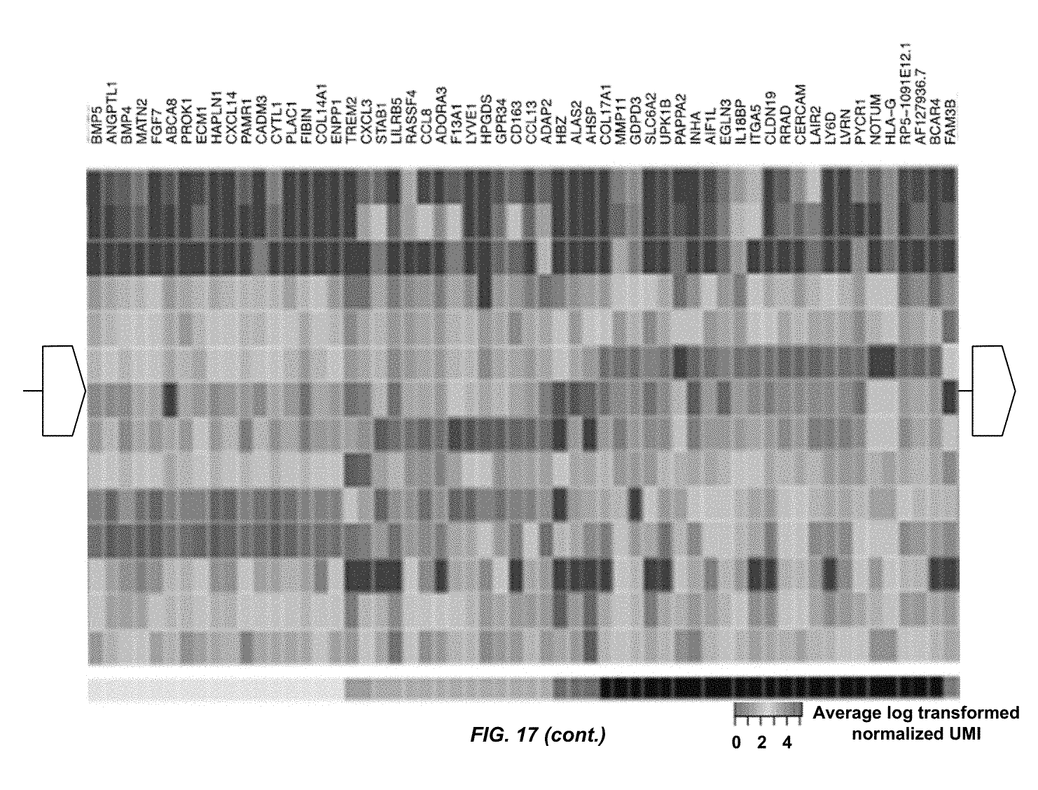

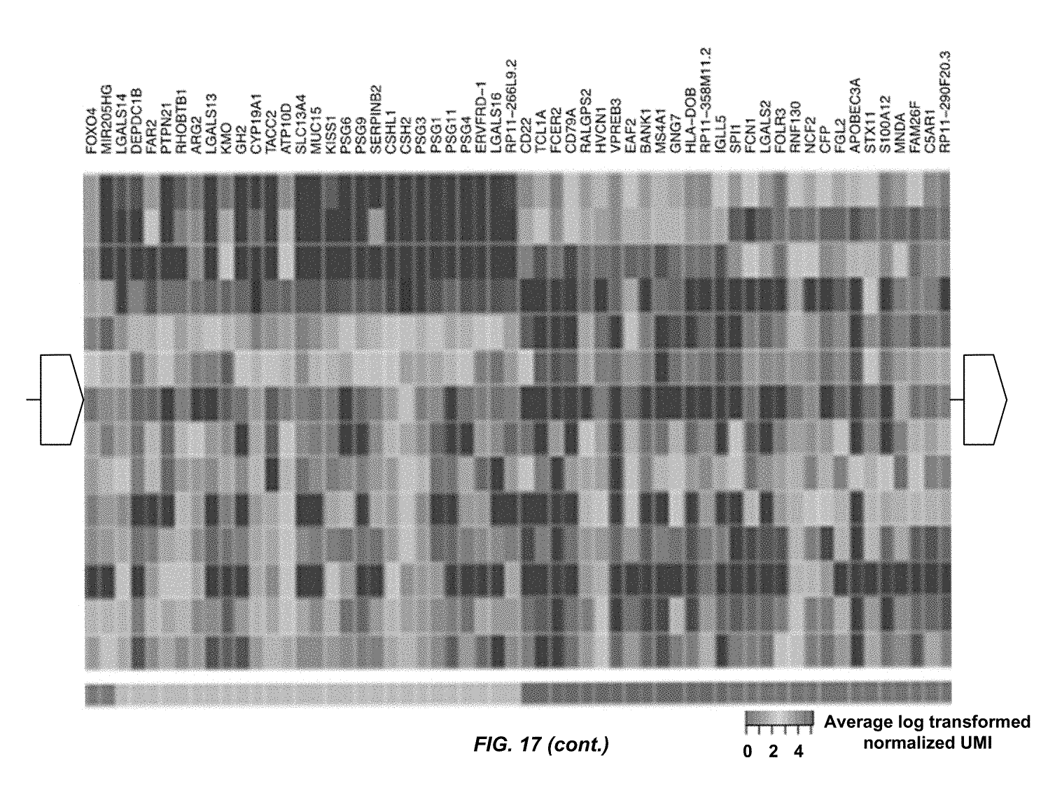

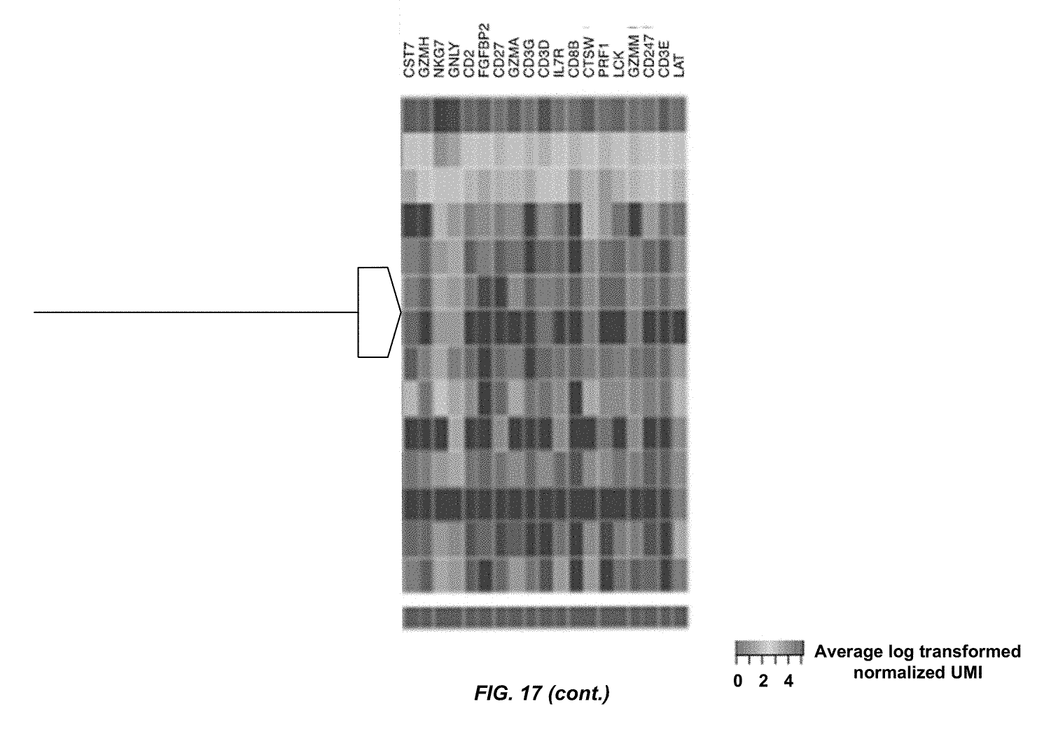

The average expression level may be a mean, median, or mode. The thresholds while listed as 0.01 and 0.1 may vary depending on a desired specificity or sensitivity. The thresholds may be chosen from 0.005, 0.01, 0.02, 0.03, 0.04, 0.05, 0.06, 0.07, 0.08, 0.09, 0.1, 0.2, 0.3, 0.4, or 0.5. Among the 14 cell clusters in the PBMC-placenta datasets, no specific genes were identified for cluster P5 and only less than 5 genes passed the filter for cluster P6, P9, and P11. Cellular dynamics analysis was not performed in these four clusters due to the low number of genes identified. Expression levels of genes in the bulk tissue profile of the placenta, liver, and leukocytes were compared to further select gene sets that showed highest expression specificity in the placenta. Genes in gene sets of placental cells and peripheral blood cells have to show the highest expression in the placenta and leukocyte bulk profiles, respectively. The bulk tissue expression datasets were retrieved online from the Human lincRNAs Catalog project (35) http://www.broadinstitute.org/genome_bio/human_lincrnas/. P7 regions were removed from further analysis due to inadequate placenta and leukocyte/liver separation (FIG. 10E). A list of genes can be found in FIG. 16 and the heat map of the genes is displayed in FIG. 17. The list of genes may be the set of preferentially expressed regions for placental cells and PBMC.

8. Signature Score Analysis

We reasoned that using single RNA transcript as marker to monitor cellular dynamics in plasma RNA will be subjected to detection variability of massively parallel RNA-sequencing due to the low levels of RNA in plasma. The problem can be improved by taking into account of multiple cell type-specific genes in a defined gene set.

We therefore measured the expression levels of individual cell type-specific signature gene sets in the plasma RNA profiles by a quantifiable composite parameter (S: Cell signature score). In one example, we computed the arithmetic mean of log 2-transformed expression level of genes in the gene set as the measure of S in the plasma RNA.

$$S = \frac{1}{n} {\sum }_{k = 1}^{n} log ( {E}_{k} + 1 )$$

S: Signature score
n: Total number of cell-specific genes in the gene set
E: Expression level of the cell-specific gene

In embodiments, the cell type-specific signature score can range from 0 to infinity, dependent on the limit of the expression levels of the constituent cell type-specific genes. Its unit is also dependent on the unit of the way that RNA expression is quantified. Nevertheless, cell type-specific signature scores of different cellular components of interest in the plasma RNA profile are not fractional representation and do not necessarily sum to 100%. This means that changes of the signature score of one particular cell type in the plasma RNA profile may not necessarily result in reciprocal changes of the signature scores of other cell types which are irrelevant in the disease of interest. The calculation of the signature score may be one way of measuring the signature score, as described in block 216 of FIG. 2.

9. Placental Cellular Dynamic Analysis

We reanalyzed the maternal plasma RNA profiles from Tsui et al (20). In additions, we generated new plasma RNA data from 2 healthy pregnant women (24-30^{th} weeks of gestation) and 2 pregnant women suffering from severe preeclampsia following the method described by Tsui et al (20). The plasma RNA profiles were normalized by size factor normalization using DESeq2 (60). The cell type-specific signature scores of each plasma RNA profile were calculated as the average normalized count levels of the specific signature gene set. The maternal plasma samples were grouped into 5 groups (A: Non-pregnant; B: Early pregnancy (13^{th}-20^{th} week); C: Mid/Late pregnancy (24^{th}-30^{th} week); D: Pre-delivery; E: 24-hours Postpartum). The average signature scores of each group were then compared as the change with respective to non-pregnant level to illustrate the cellular dynamics in pregnancy progression. Alternatively, maternal plasma RNA-seq profiles of Koh et al (21) were retrieved from SRP042027. The data were aligned using STAR (59). Cases with mappable reads >1,000,000 and samples across four different time points (1^{st} trimester, 2^{nd} trimester, 3^{rd} trimester and 6 weeks postpartum) were selected for further analysis (Case 2, 15, 24 and 32). The average signature scores in each group were calculated as described above. The change is then visualized as the change with respective to first-trimester pregnant women level. Dynamics of P4 (Stromal cells) was not analyzed due to low number (<50%) of signature genes detected in the plasma profiles.

10. Placental Cellular Signature Expression Comparison in PET and Normal Maternal Plasma

The maternal plasma RNA levels of different cell type-specific signatures were compared between group C (Mid/Late pregnancy plasma) and 2 preeclampsia toxaemia (PET) patients (data shown in FIG. 14A). A new cohort of 5 PET patients and 8 healthy third-trimester pregnant women were recruited to validate the finding of differential EVTB cell signature expression in the Tsui dataset. In this new cohort, the plasma RNA profiles were generated using the Ovation RNA-Seq System V2 (NuGEN) similar to that of Koh et al (21) and analyzed as described above. The statistical significance of the differences of EVTB signature between PET and healthy controls were determined by two-tailed two-sample Wilcoxon signed rank test.

11. Microarray Genotyping and Single Nucleotide Polymorphism (SNP) Identification

Genomic DNA extracted from maternal buffy coat and placental tissue biopsies was genotyped with the Infinium Omni2.5-8 V1.2 Kit and the iScan system (Illumina). SNP calling were performed using the Birdseed v2 algorithm. The fetal genotypes of the placentas were compared with the maternal buffy coat genotypes to identify the fetal-specific SNP alleles. A SNP was considered as informative if it was homozygous in the mother and heterozygous in the fetus.

12. Statistical Analysis

Details of statistical analyses were described in the corresponding section above. We regard a P-value less than 0.05 as statistically significant.

### III. INTEGRATIVE SINGLE-CELL AND CELL-FREE PLASMA RNA ANALYSIS FOR CANCER AND SLE

The integrative single-cell and cell-free plasma RNA analysis described for pregnancy and preeclampsia can be applied to conditions that may not be related to pregnancy. For example, the analysis can be used to determine expressed markers for systemic lupus erythematosus (SLE) and cancer.

A. Detecting Blood Cell Aberrations in Autoimmune Systemic Lupus Erythematosis (SLE)

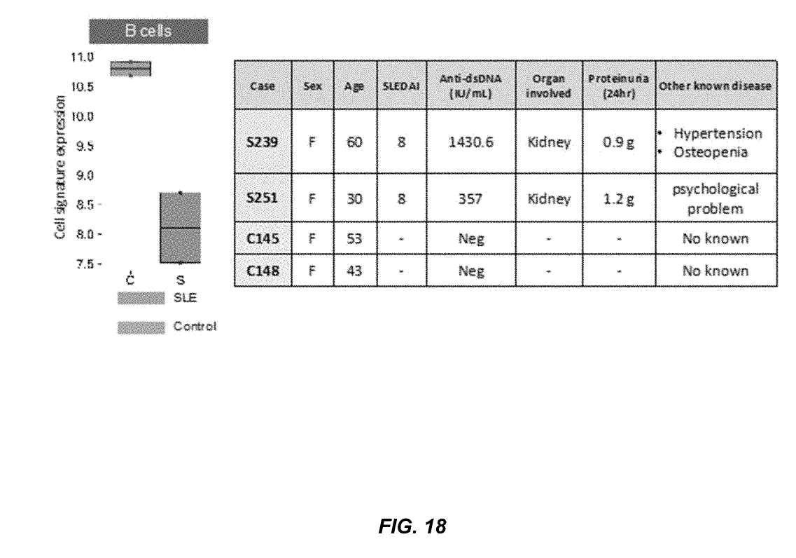

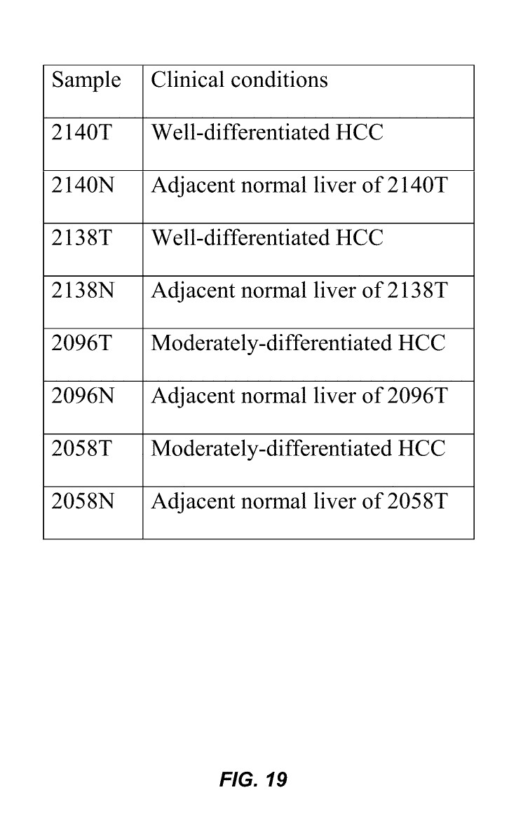

In another example, we demonstrated that this analytical approach can be used to reveal the cellular aberrations of other biological systems in non-gestational diseases. In this exemplification, we studied the plasma cell-free RNA profiles of two patients suffering from systemic erythematosus (SLE), recruited from the Department of Medicine and Therapeutics, Prince of Wales Hospital, Hong Kong. Both of them have presence of anti-dsDNA antibodies in the circulation and proteinuria. Placenta cells and PBMC cells were used for this analysis. We showed that the B-cell specific signature levels discovered in our previous analysis is consistently reduced in SLE patients (FIG. 18). This is consistent with the fact that B cell abnormalities have been recognized as the major pathological mechanism in SLE (28).

B. Detecting Liver Cancer in Hepatitis B Virus Infected Patients

In another example, we demonstrated application in the detection and monitoring of treatment in cancer patients. As an exemplification, we profiled the single-cell RNA transcriptome profiles non-marker selected cells from 4 tumor resection biopsies of HBV-related hepatocellular carcinoma (HCC) and their adjacent non-tumorous tissues (Sample 2140, 2138, 2096 and 2058). FIG. C21 shows the sample name and the clinical conditions for the sample.

The tumor and non-tumor liver tissues were washed by PBS buffer, and were dissociated by 0.5% collagenase A (Sigma Aldrich) digestion for about 1 hour at 37 degree Celsius. The tissues were gently triturated and filtered by 100 μm strainer (Miltenyi Biotech) to remove large debris. Red blood cells were lysed by ACK buffer (Invitrogen) for 1 minute in room temperature and the cells were washed again using hepatocyte washing medium (Thermo Fisher Scientific) before final filtering with 70 μm strainer (Miltenyi Biotech). Successful dissociation was confirmed under a microscope.

Single cell transcriptomic libraries were generated using the Chromium Single Cell 3′ Library & Gel Bead Kit v2 (10× Genomics). Cells were loaded into a Single Cell 3′ Chip (10× Genomics), about 4000 cells were aimed for targeted cell recovery per sample. RNA transcripts from single cells were uniquely barcoded and reverse transcribed within droplets. cDNA molecules were pre-amplified and pooled followed by library construction according to protocol instruction. All libraries were quantified by Qubit and real-time quantitative PCR on a LightCycler 96 System (Roche). The size profiles of the pre-amplified cDNA and sequencing libraries were examined by the Agilent High Sensitivity D5000 and High Sensitivity D1000 ScreenTape Systems (Agilent), respectively. The libraries were sequenced on massively parallel sequencer (HiSeq2500, Illumina). Sequencing reads were mapped to the human reference genome and gene expression quantification as number of unique molecular identifiers (UMIs) were performed using the Cell Ranger pipeline version 2.0 by 10× Genomics.

To remove poor quality cells from the data after Cell Ranger pipeline processing, we removed cells which showed no expression of the housekeeping gene ACTB, or cells with fraction of total UMI count originating from mitochondria-encoded genes >20%, or cells with total UMI counts below the 5^{th} percentile or above the 95^{th} percentile in their sample of origin, or cells with number of genes below the 5^{th} percentile or above the 95^{th} percentile in their sample of origin. Principal component analysis was performed and the first 5 principal components, which captured the most significant variation in the dataset, were selected for two dimensional t-stochastic neighborhood embedding.

Based on proximity of cells in the t-SNE projection and expression of know cell markers, we annotated the biological identity of the cells into six cell groups for cell-type specific marker discovery: Hepatocyte-like cells, cholangiocyte-like cells, myofibroblast-like cells, endothelial cells, lymphoid cells, and myeloid cells.

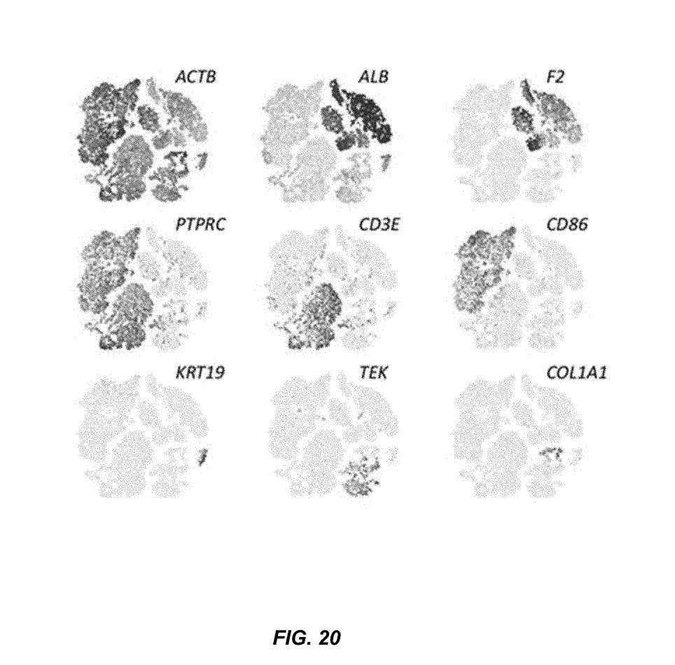

FIG. 20 shows the expression pattern of selected genes (titled in each panel) that are known to be specific to certain types of cells in the human liver (expression quantified as log-transformed UMI counts). Each dot in the plot represents the transcriptomic data from a single cell. Grey color indicates no expression, and the brighter the shades of orange-red indicates the higher levels of expression.

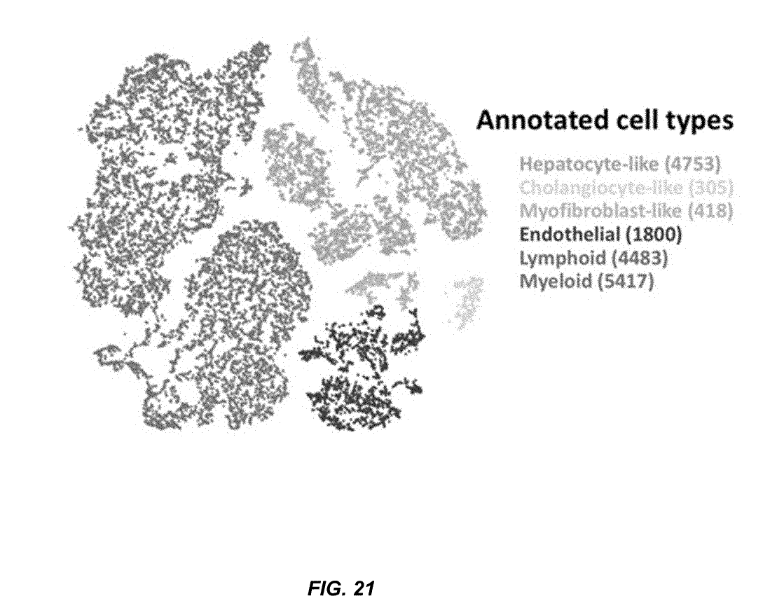

FIG. 21 shows computational single-cell transcriptomic clustering pattern of HCC and adjacent non-tumor liver cells by PCA-t-SNE visualization. Each dot in the plot represents the transcriptomic data from a single cell, the proximity of each dot is related to similarities in RNA expression profiles. The clusters are further colored and grouped into 6 subgroups based on spatial proximity and expression pattern of known cell type-specific marker expression as noted in FIG. 20. The numbers in bracket indicates the number of cells in corresponding cell types.

In this example, we selected cell type-specific genes again using Z score statistics as the difference threshold (Z>=3), normalized UMI counts <0.2/cell type as the maximum threshold in comparative cell types and normalized UMI counts >=1 UMI/cell type as the minimal threshold in the testing cell group.
1. Genes with expression z score greater than 3, AND

Gene expression z scores are calculated as:

$${z}_{g} = \frac{{g}_{A} - {g}_{\overline{A}}}{{s}_{\overline{A}}}$$

z_{g}: z score for gene g
g_{A}: average expression level of gene g in testing cell type A (normalized UMI count)
g_{Ā}: mean of the average expression level of gene g in other non-A comparative cell types (normalized UMI count)
s_{Ā}: standard deviation of the average expression in other non-A comparative cell types.
2. Average expression levels (normalized UMI) in testing cell type greater than threshold (>=1 UMI/cell), AND
3. Average expression levels (normalized UMI) in other comparative cell types less than threshold (<0.2 UMI/cell type)

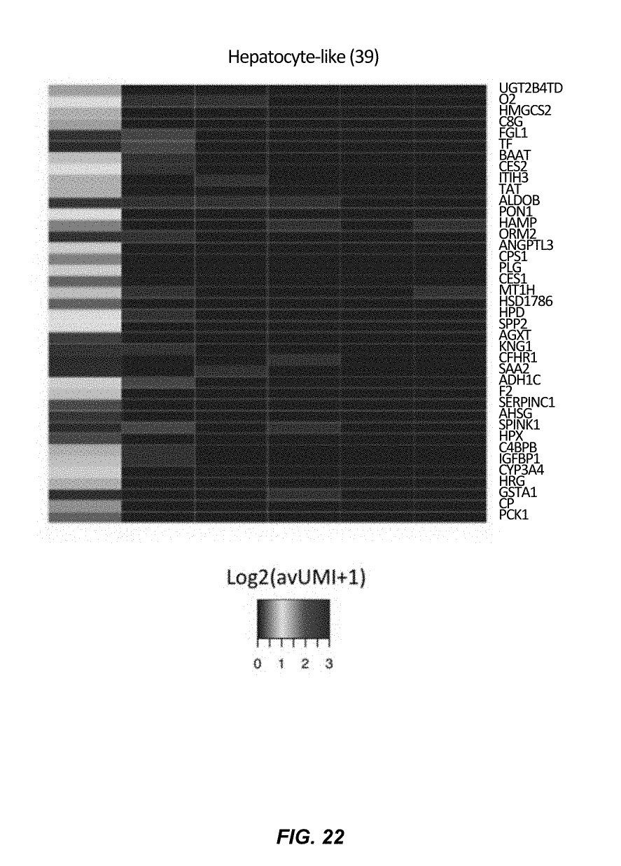

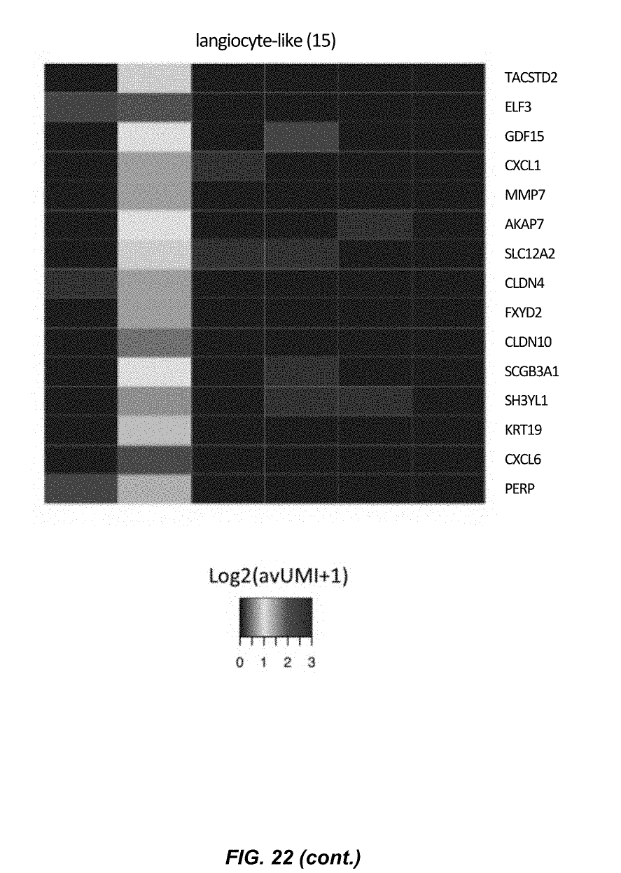

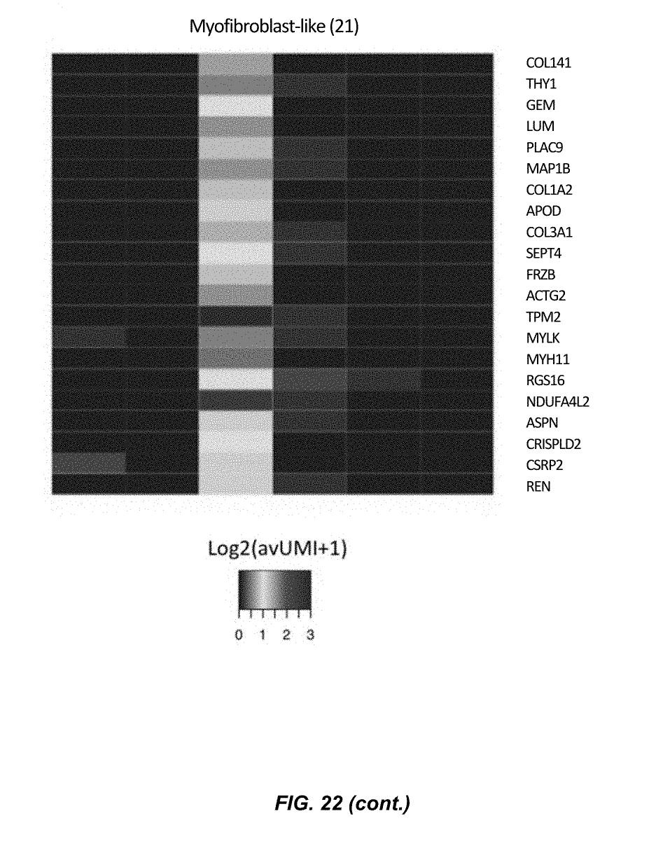

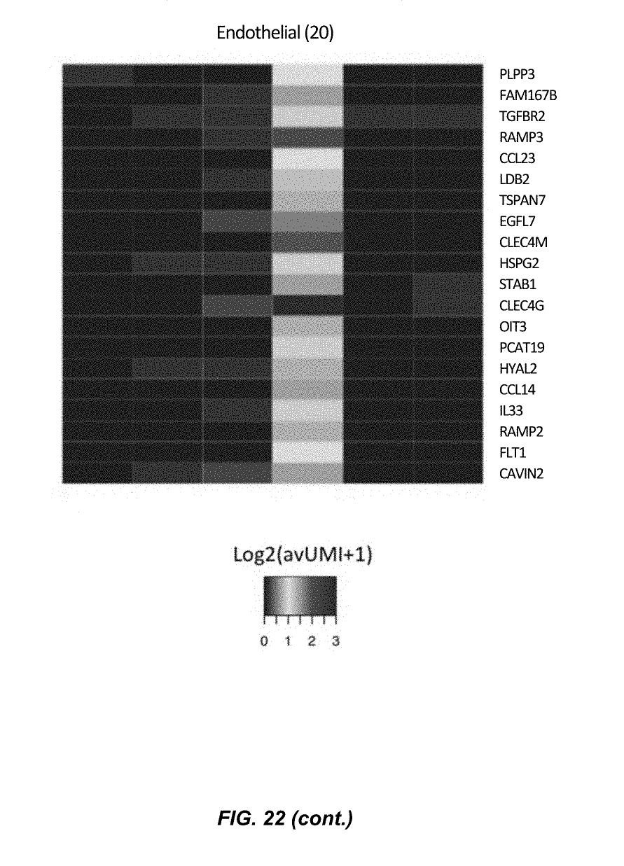

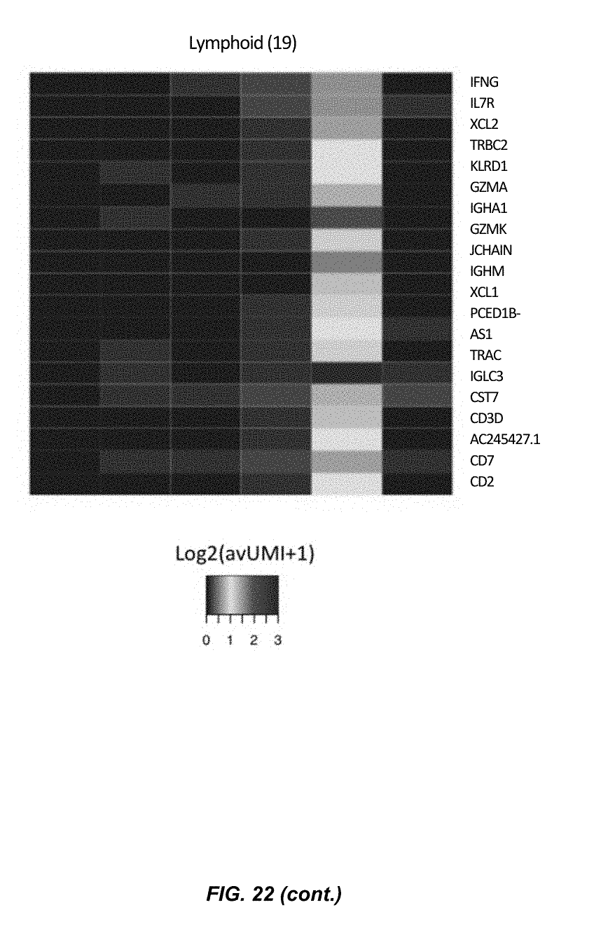

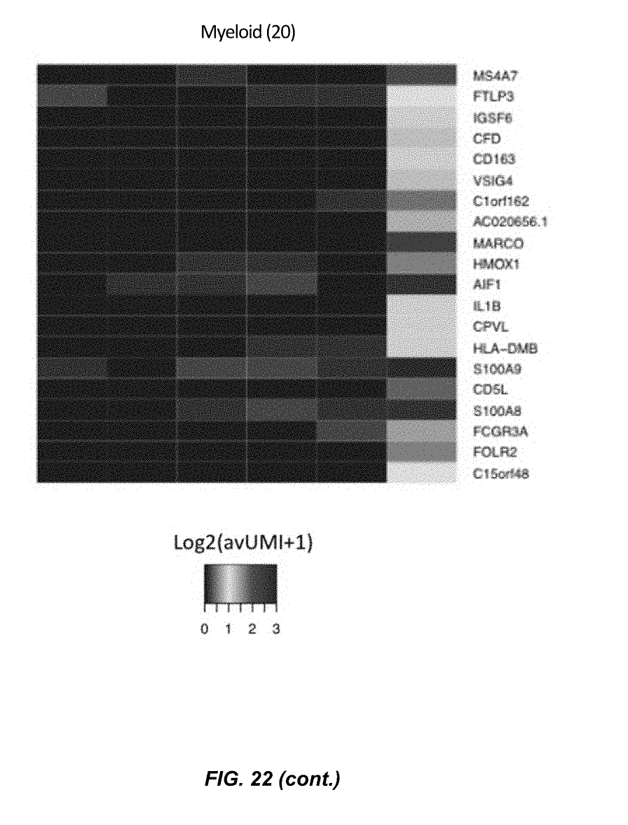

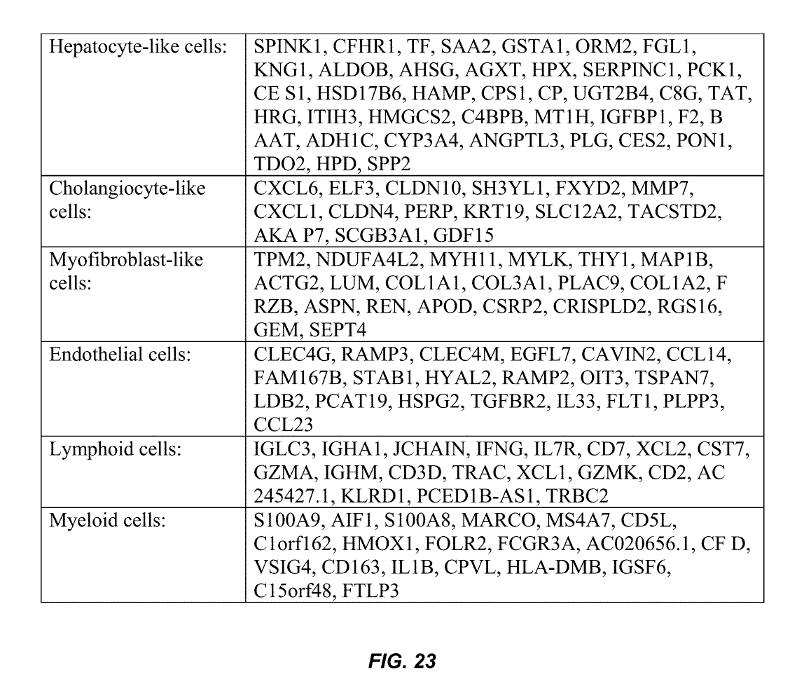

FIG. 22 shows identification of cell type-specific genes in the HCC/liver single-cell RNA transcriptomic dataset. Cell type-specific genes of each annotated cell types were presented in expression heat maps. The numbers in bracket indicate the total number of cell type-specific genes in the corresponding cell type. FIG. 23 shows a listing of the cell type-specific genes. Any of the genes in the listing may be in the set of one or more preferentially expressed regions.

Comparisons with whole-tissue or single-cell expression profiles of other human organs/tissues, e.g. placenta and PBMC, were not necessarily required in this example, since the patient is non-pregnant and the HCC/liver single-cell RNA transcriptomic dataset already contained the two major groups of blood cells (lymphoid and myeloid cells).

We then demonstrated the utility of the cell type-specific gene sets in the detection and monitoring of patients with hepatocellular carcinoma and chronic hepatitis B with or without cirrhosis.

In this example, we recruited and analyzed the plasma RNA profiles of healthy controls (n=8), patients with hepatitis B virus (HBV) infection and cirrhosis (n=23), patients with hepatitis B virus (HBV) infection and no cirrhosis (n=18), patients with hepatitis B virus (HBV)-associated hepatocellular carcinoma (n=12) and patients received HBV-associated hepatocellular resection surgery 24-hour prior (n=7). Chronic HBV infection is defined by the presence of hepatitis B virus surface antigen (HBsAg) and cirrhosis is defined by ultrasound imaging evidence. The plasma RNA samples were processed as described similar to the maternal plasma samples.

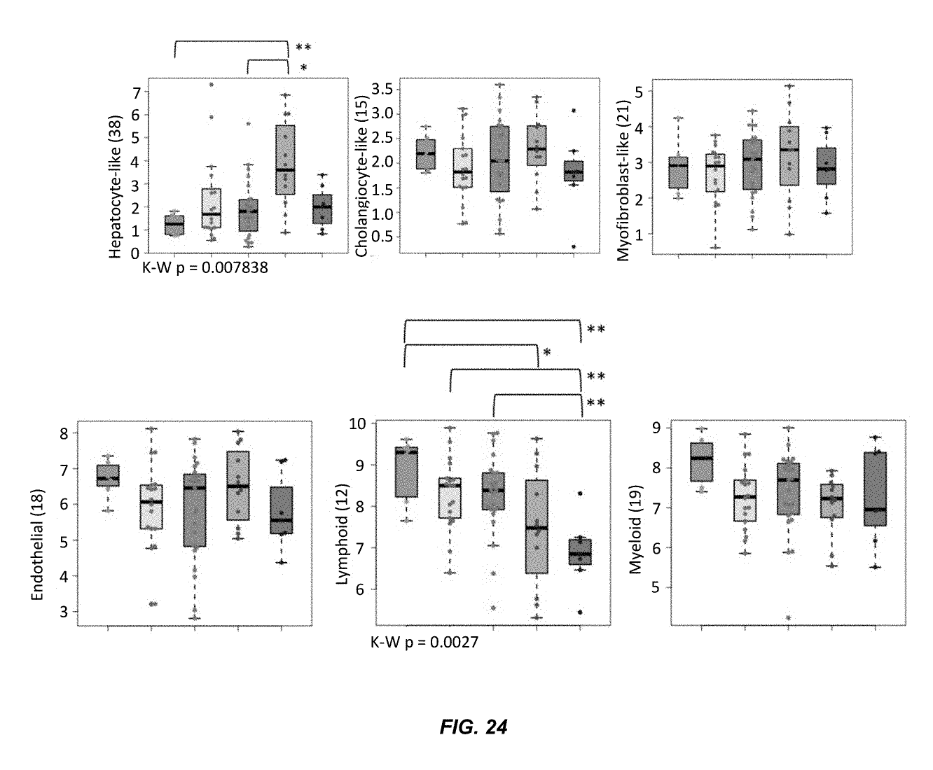

FIG. 24 shows a comparison of cell signature scores of different cell types in plasma samples (Left to right) from healthy controls, chronic HBV without cirrhosis, chronic HBV with cirrhosis and HCC pre-operation and HCC post-operation patients. Kruskal-Wallis test by ranks was performed for non-parametric analysis of variance and two-sample two-tailed Wilcoxon signed rank tests were performed to test for statistical significance between sample groups in cell types showing statistical significance (K-W p<0.05). The p values were adjusted for multiple testing by Benjamini-Hochberg method *p<0.05, **p<0.01. The Y-axis denotes the cell signature scores computed as described. The numbers in bracket indicate the total number of cell type-specific genes in the corresponding cell type.

Comparisons of signature scores of each cell types in the plasma RNA profiles showed that the hepatocyte-like cell signature is significantly elevated in patients with confirmed hepatocellular carcinoma compared to other patient groups. The signal is reduced in HCC patients 24 hours after tumor resection. In contrast, lymphoid cell signature score is reduced significantly in patient with HCC compared to healthy controls.

In another example, we demonstrated that analysis combining more than one cell signature scores can improve differentiation of HBV-related HCC patients from non-HCC HBV patients by plasma RNA analysis. Chan et al previously showed that targeted detection of a single liver-specific transcript, ALB, in plasma RNA by real-time quantitative PCR assay can be utilized to detect liver pathology, such as transplant monitoring, HCC and cirrhosis (30). We therefore compared the diagnostic performance of ALB transcript detection and plasma RNA cell-type specific signature score measurement in differentiation of HBV-related HCC patients from non-HCC HBV patients with and without cirrhosis.

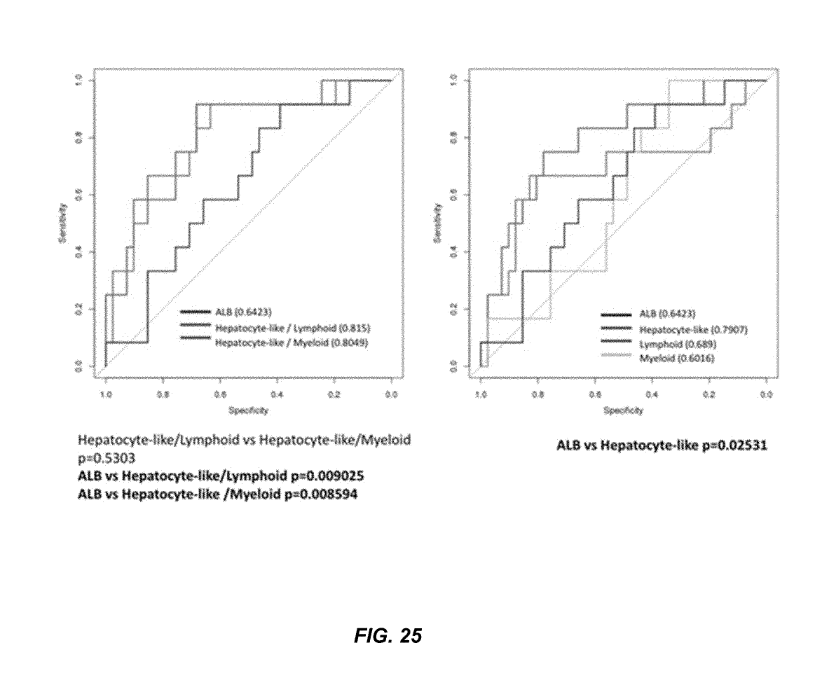

FIG. 25 shows receiver operating characteristic curves of different approaches in the differentiation of non-HCC HBV (with or without cirrhosis) versus HBV-HCC patients. The left panel shows comparison of performance using the level of single liver-specific transcript ALB in plasma, ratio of hepatocyte-like to lymphoid cell signature score, and ratio of hepatocyte-like to myeloid cell signature score. The right panel compared the diagnostic performance of ALB alone, hepatocyte-like alone, lymphoid alone, and myeloid alone signature scores. The numbers in bracket denote the area under curve. Thep values by DeLong's test is given.

Receiver operating characteristics curve analysis showed that cell type-specific signature score of hepatocyte-like cells (0.7907) has higher area under curve than ALB transcript (0.6423) (DeLong's test p=0.02531). The area under curve is further increased if the ratio of hepatocyte-like cells to lymphoid cells (0.815) or the ratio of hepatocyte-like cells to myeloid cells (0.8049) is used. These results suggested that the mathematical transformation of the quantitative relationship of different cell type-specific signatures can be utilized to improve plasma RNA diagnostics.

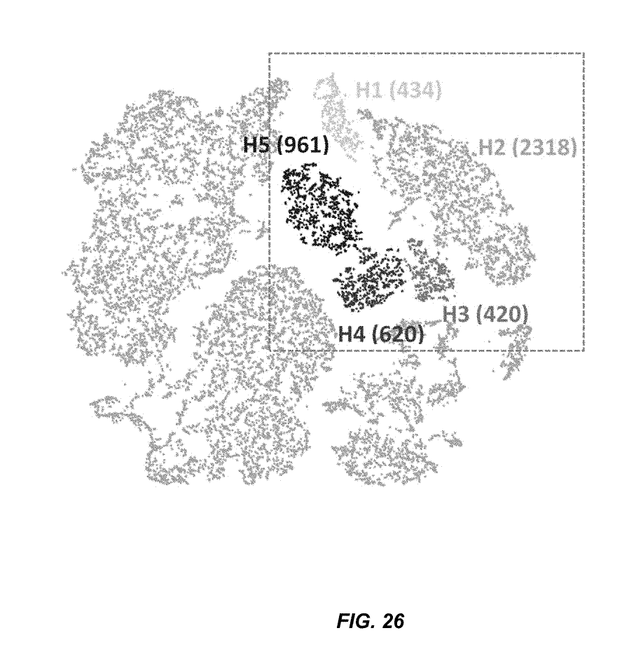

In another example, we further separated the hepatocyte-like cell group into 5 subgroups (H1-5) based on clustering pattern on t-SNE projection, as shown in FIG. 26. In FIG. 26, the numbers in the brackets represent the number of cells in each subgroup. FIG. 26 is based on the same cells that were in FIG. 21. The hepatocyte-like cluster in FIG. 21 by the spatial pattern that subgroups may be present. In addition, we expected that the hepatocyte cells may include both normal liver cells and tumor cells.

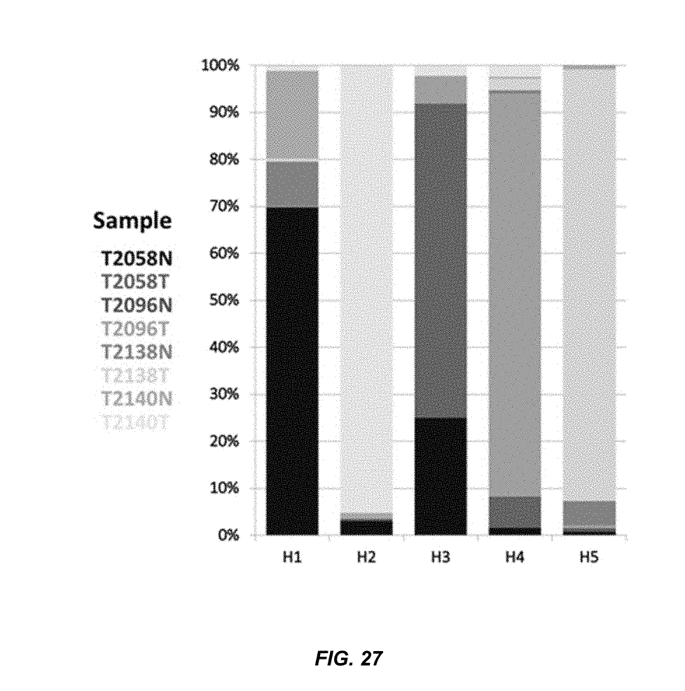

FIG. 27 shows the origin of cells in the five subgroups. Analysis of the library origins of cells showed that H1 is composed of cells from adjacent non-tumor liver tissues primarily. H2, H3, H4, and H5 are dominated by cells from tumor tissues of the four tissue donors individually.

Division of other clusters into subgroups or subgroups into further subgroups may be possible. The decision to analyze subgroups may depend on prior knowledge regarding the tissues (e.g., biological hypothesis driven) and/or statistical analysis (e.g., k-mean statistics).

For example, in tumor single cell RNA results, we expect at least six hidden cell types including infiltrating lymphoid cells and myeloid cells, normal liver cells, tumor cells, endothelial cells, and cholangiocyte cells. Thus, we try to locate six clusters first with the use of k-mean clustering results plus the expression patterns of known markers. Once we saw the elevated signal of hepatic clusters in plasma RNA results, then we decide to further subtype the hepatic cluster according to shapes of sub-clusters shown in the 2D t-SNE plot because we expected that tumor cells would be present in the hepatic cluster. There were five sub-sub groups present in hepatic clusters showing relatively clear boundaries.

Alternatively, we can use some statistics approaches to determine the number of clusters which should be taken into account. For example, (1) we can stop look into the subgroups of subgroups when the total intra-cluster variation is minimized. The total intra-cluster variation reflects the compactness of the clustering which are supposed to be minimized (ref. Kaufman, L. and P. J. Rousseeuw, Finding Groups in Data (John Wiley & Sons, New York, 1990); (2) the optimal number of clusters could be the one that maximize average silhouette (Peter J. Rousseeuw (1987). “Silhouettes: a Graphical Aid to the Interpretation and Validation of Cluster Analysis.” Computational and Applied Mathematics. 20: 53-65); (3) the optimal number of clusters could also the one that maximizes the gap statistic (R. Tibshirani, G. Walther, and T. Hastie (Stanford University, 2001). http://web.stanford.edu/˜hastie/Papers/gap.pdf). The gap statistic is used to mean the deviation in intra-cluster variation between the reference data set with a random uniform distribution (computational simulation) and observed clusters.

Cell subgroup-specific genes identification of the H1-H5 subgroups using Z score statistics as the difference threshold (Z>=3), normalized UMI counts <0.5/cell type as the maximum threshold in comparative cell types and normalized UMI counts >=1 UMI/cell type as the minimal threshold in the testing cell group identified 16 H1-H5 specific genes.

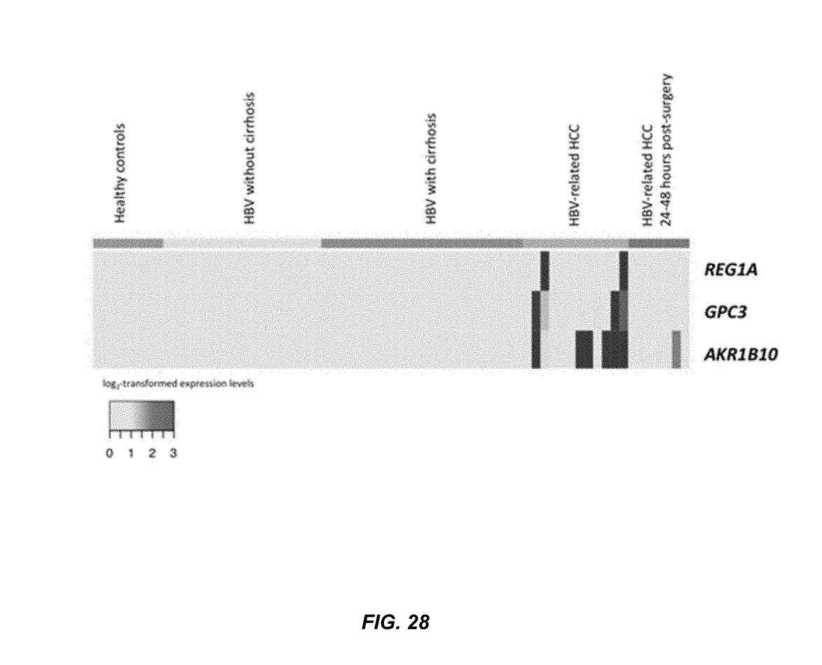

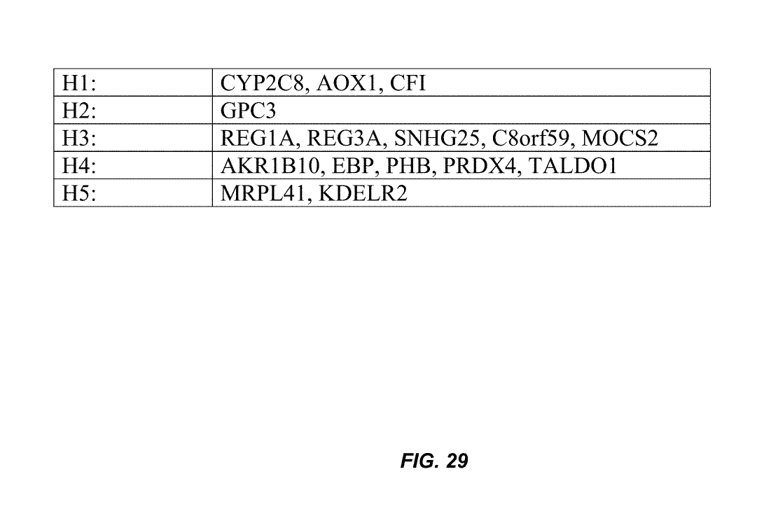

FIG. 28 is an expression heat map showing the expression of H2 subgroup-specific gene GPC3, H3 subgroup-specific gene REG1A, and H4-subgroup specific gene AKR1B10 in the plasma RNA profile of healthy controls, patients of HBV without cirrhosis, patients of HBV with cirrhosis, patients of HBV-related HCC and patients received HCC resection surgery 24-48 hours prior. We found that 3 genes (REG1A, GPC3 and AKR1B10) are specifically expressed in the plasma RNA of HCC patients before surgery, completely absent in healthy controls and absent in non-HCC HBV patients with or without cirrhosis (specificity=100%, 49/49). Combining detection of all three genes, the sensitivity of HCC detection is 66.67% (8/12). FIG. 29 shows the list of subgroup-specific genes.

### IV. CONCLUSION

We illustrated the concept of cellular information derivation from acellular materials, such as plasma RNA, using single-cell RNA transcriptomic information of the tissue of interest. A quantitative signature scores can be computed based on the expression levels of certain RNA transcripts in the plasma which were selected based on cell type-specificity identified in single-cell RNA transcriptomic dataset of the source tissue to detect pathology and monitor the change of the source tissues. We illustrated this using pregnancy progression, detection of severe early preeclampsia, autoimmune systemic lupus erythematosus and liver cancer as examples. It is applicable in subtyping of disease such as separation of non-HCC HBV infection and HBV-related HCC patients, and treatment outcome using changes of pre-operative and post-operative patients with liver cancer resection as example.

This approach can be expanded to genomic and epigenomic analysis in cell-free DNA analysis, where cell type-specific genomic mutations or cell type-specific epigenomic changes, for example, DNA methylation, histone modifications, can be first defined at the single-cell level in the tissue of interest and be quantified in the cell-free DNA profile.

### V. EXAMPLE SYSTEMS

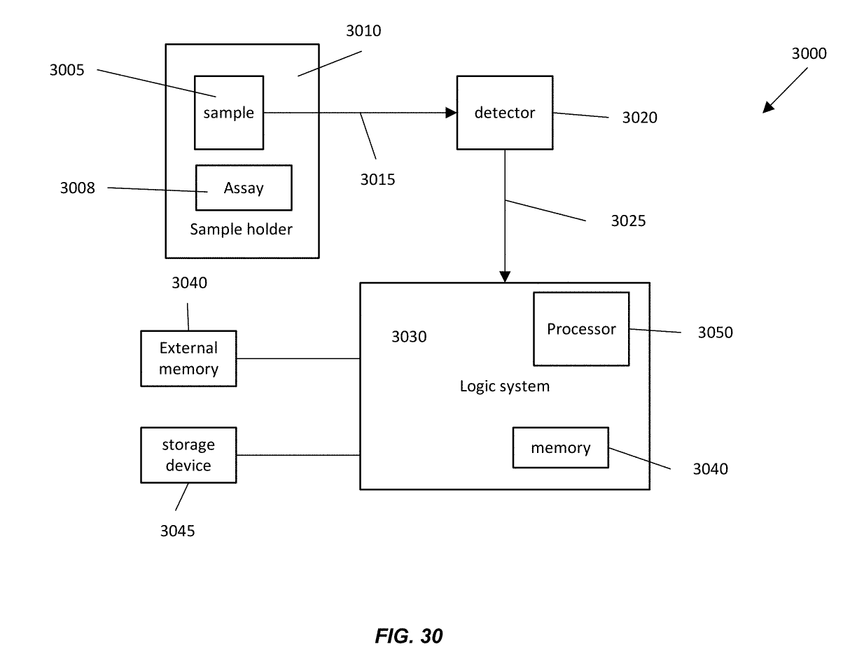

FIG. 30 illustrates a system 3000 according to an embodiment of the present invention. The system as shown includes a sample 3005, such as cell-free DNA molecules within a sample holder 3010, where sample 3005 can be contacted with an assay 3008 to provide a signal of a physical characteristic 3015. In some embodiments, sample 3005 may be a single cell with nucleic acid material. An example of a sample holder can be a flow cell that includes probes and/or primers of an assay or a tube through which a droplet moves (with the droplet including the assay). Physical characteristic 3015, such as a fluorescence intensity value, from the sample is detected by detector 3020. Detector can take a measurement at intervals (e.g., periodic intervals) to obtain data points that make up a data signal. In one embodiment, an analog to digital converter converts an analog signal from the detector into digital form at a plurality of times. A data signal 3025 is sent from detector 3020 to logic system 3030. Data signal 3025 may be stored in a local memory 3035, an external memory 3040, or a storage device 3045.

Logic system 3030 may be, or may include, a computer system, ASIC, microprocessor, etc. It may also include or be coupled with a display (e.g., monitor, LED display, etc.) and a user input device (e.g., mouse, keyboard, buttons, etc.). Logic system 3030 and the other components may be part of a stand-alone or network connected computer system, or they may be directly attached to or incorporated in a thermal cycler device. Logic system 3030 may also include optimization software that executes in a processor 3050.

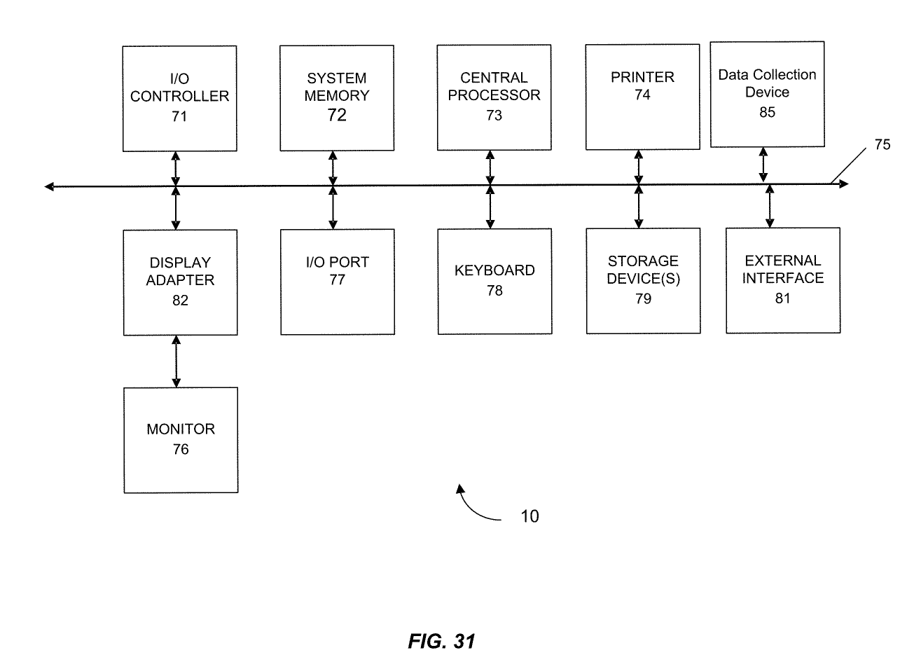

Any of the computer systems mentioned herein may utilize any suitable number of subsystems. Examples of such subsystems are shown in FIG. 31 in computer apparatus 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones, and other mobile devices.

The subsystems shown in FIG. 31 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of connections known in the art such as input/output (I/O) port 77 (e.g., USB, FireWire). For example, I/O port 77 or external interface 81 (e.g. Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware (e.g. an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the operations. Thus, embodiments can be directed to computer systems configured to perform the operations of any of the methods described herein, potentially with different components performing a respective operations or a respective group of operations. Although presented as numbered operations, operations of methods herein can be performed at a same time or in a different order. Additionally, portions of these operations may be used with portions of other operations from other methods. Also, all or portions of an operation may be optional. Additionally, any of the operations of any of the methods can be performed with modules, units, circuits, or other approaches for performing these operations.

The section headings used herein are for organizational purposes only and are not to be construed as limiting the subject matter described.

It is to be understood that the methods described herein are not limited to the particular methodology, protocols, subjects, and sequencing techniques described herein and as such may vary. It is also to be understood that the terminology used herein is for the purpose of describing particular embodiments only, and is not intended to limit the scope of the methods and compositions described herein, which will be limited only by the appended claims. While some embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

Several aspects are described with reference to example applications for illustration. Unless otherwise indicated, any embodiment can be combined with any other embodiment. It should be understood that numerous specific details, relationships, and methods are set forth to provide a full understanding of the features described herein. A skilled artisan, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

While some embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention.

Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a method” includes a plurality of such methods and reference to “the particle” includes reference to one or more particles and equivalents thereof known to those skilled in the art, and so forth. The invention has now been described in detail for the purposes of clarity and understanding. However, it will be appreciated that certain changes and modifications may be practice within the scope of the appended claims.

### VI. REFERENCES

All patents, patent applications, publications, and descriptions mentioned herein are incorporated by reference in their entirety for all purposes. None is admitted to be prior art.
1. G. J. Burton, A. L. Fowden, The placenta: a multifaceted, transient organ. Philos Trans R Soc Lond B Biol Sci 370, 20140066 (2015).
2. T. Chaiworapongsa, P. Chaemsaithong, L. Yeo, R. Romero, Pre-eclampsia part 1: current understanding of its pathophysiology. Nat Rev Nephrol 10, 466-480 (2014).
3. S. J. Fisher, Why is placentation abnormal in preeclampsia? Am J Obstet Gynecol 213, S115-122 (2015).
4. A. M. Vintzileos, C. V. Ananth, J. C. Smulian, Using ultrasound in the clinical management of placental implantation abnormalities. Am J Obstet Gynecol 213, S70-77 (2015).
5. H. Zeisler, E. Llurba, F. Chantraine, M. Vatish, A. C. Staff, M. Sennstrom, M. Olovsson, S. P. Brennecke, H. Stepan, D. Allegranza, P. Dilba, M. Schoedl, M. Hund, S. Verlohren, Predictive Value of the sFlt-1:P1GF Ratio in Women with Suspected Preeclampsia. N Engl J Med 374, 13-22 (2016).
6. S. S. Chim, Y. K. Tong, R. W. Chiu, T. K. Lau, T. N. Leung, L. Y. Chan, C. B. Oudejans, C. Ding, Y. M. Lo, Detection of the placental epigenetic signature of the maspin gene in maternal plasma. Proc Natl Acad Sci USA 102, 14753-14758 (2005).
7. M. Alberry, D. Maddocks, M. Jones, M. Abdel Hadi, S. Abdel-Fattah, N. Avent, P. W. Soothill, Free fetal DNA in maternal plasma in anembryonic pregnancies: confirmation that the origin is the trophoblast. Prenat Diagn 27, 415-418 (2007).
8. B. H. Faas, J. de Ligt, I. Janssen, A. J. Eggink, L. D. Wijnberger, J. M. van Vugt, L. Vissers, A. Geurts van Kessel, Non-invasive prenatal diagnosis of fetal aneuploidies using massively parallel sequencing-by-ligation and evidence that cell-free fetal DNA in the maternal plasma originates from cytotrophoblastic cells. Expert Opin Biol Ther 12 Suppl 1, S19-26 (2012).
9. Y. M. Lo, T. N. Leung, M. S. Tein, I. L. Sargent, J. Zhang, T. K. Lau, C. J. Haines, C. W. Redman, Quantitative abnormalities of fetal DNA in maternal serum in preeclampsia. Clin Chem 45, 184-188 (1999).
10. E. K. Ng, T. N. Leung, N. B. Tsui, T. K. Lau, N. S. Panesar, R. W. Chiu, Y. M. Lo, The concentration of circulating corticotropin-releasing hormone mRNA in maternal plasma is increased in preeclampsia. Clin Chem 49, 727-731 (2003).
11. A. Martin, I. Krishna, M. Badell, A. Samuel, Can the quantity of cell-free fetal DNA predict preeclampsia: a systematic review. Prenat Diagn 34, 685-691 (2014).
12. Y. G. Zhang, H. L. Yang, Y. Long, W. L. Li, Circular RNA in blood corpuscles combined with plasma protein factor for early prediction of pre-eclampsia. BJOG 123, 2113-2118 (2016).
13. T. N. Leung, J. Zhang, T. K. Lau, N. M. Hjelm, Y. M. D. Lo, Maternal plasma fetal DNA as a marker for preterm labour. The Lancet 352, 1904-1905 (1998).
14. A. Farina, E. S. LeShane, R. Romero, R. Gomez, T. Chaiworapongsa, N. Rizzo, D. W. Bianchi, High levels of fetal cell-free DNA in maternal serum: a risk factor for spontaneous preterm delivery. Am J Obstet Gynecol 193, 421-425 (2005).
15. T. R. Jakobsen, F. B. Clausen, L. Rode, M. H. Dziegiel, A. Tabor, High levels of fetal DNA are associated with increased risk of spontaneous preterm delivery. Prenat Diagn 32, 840-845 (2012).
16. Y. Y. Lui, K. W. Chik, R. W. Chiu, C. Y. Ho, C. W. Lam, Y. M. Lo, Predominant hematopoietic origin of cell-free DNA in plasma and serum after sex-mismatched bone marrow transplantation. Clin Chem 48, 421-427 (2002).
17. N. B. Tsui, S. S. Chim, R. W. Chiu, T. K. Lau, E. K. Ng, T. N. Leung, Y. K. Tong, K. C. Chan, Y. M. Lo, Systematic micro-array based identification of placental mRNA in maternal plasma: towards non-invasive prenatal gene expression profiling. J Med Genet 41, 461-467 (2004).
18. F. M. Lun, R. W. Chiu, K. Sun, T. Y. Leung, P. Jiang, K. C. Chan, H. Sun, Y. M. Lo, Noninvasive prenatal methylomic analysis by genomewide bisulfite sequencing of maternal plasma DNA. Clin Chem 59, 1583-1594 (2013).
19. X. Huang, T. Yuan, M. Tschannen, Z. Sun, H. Jacob, M. Du, M. Liang, R. L. Dittmar, Y. Liu, M. Liang, M. Kohli, S. N. Thibodeau, L. Boardman, L. Wang, Characterization of human plasma-derived exosomal RNAs by deep sequencing. BMC Genomics 14, 319 (2013).
20. N. B. Tsui, P. Jiang, Y. F. Wong, T. Y. Leung, K. C. Chan, R. W. Chiu, H. Sun, Y. M. Lo, Maternal plasma RNA sequencing for genome-wide transcriptomic profiling and identification of pregnancy-associated transcripts. Clin Chem 60, 954-962 (2014).
21. W. Koh, W. Pan, C. Gawad, H. C. Fan, G. A. Kerchner, T. Wyss-Coray, Y. J. Blumenfeld, Y. Y. El-Sayed, S. R. Quake, Noninvasive in vivo monitoring of tissue-specific global gene expression in humans. Proc Natl Acad Sci USA 111, 7361-7366 (2014).
22. K. Sun, P. Jiang, K. C. Chan, J. Wong, Y. K. Cheng, R. H. Liang, W. K. Chan, E. S. Ma, S. L. Chan, S. H. Cheng, R. W. Chan, Y. K. Tong, S. S. Ng, R. S. Wong, D. S. Hui, T. N. Leung, T. Y. Leung, P. B. Lai, R. W. Chiu, Y. M. Lo, Plasma DNA tissue mapping by genome-wide methylation sequencing for noninvasive prenatal, cancer, and transplantation assessments. Proc Natl Acad Sci USA 112, E5503-5512 (2015).
23. Y. Qin, J. Yao, D. C. Wu, R. M. Nottingham, S. Mohr, S. Hunicke-Smith, A. M. Lambowitz, High-throughput sequencing of human plasma RNA by using thermostable group II intron reverse transcriptases. RNA 22, 111-128 (2016).
24. M. W. Snyder, M. Kircher, A. J. Hill, R. M. Daza, J. Shendure, Cell-free DNA Comprises an In Vivo Nucleosome Footprint that Informs Its Tissues-Of-Origin. Cell 164, 57-68 (2016).
25. K. C. Chan, P. Jiang, K. Sun, Y. K. Cheng, Y. K. Tong, S. H. Cheng, A. I. Wong, I. Hudecova, T. Y. Leung, R. W. Chiu, Y. M. Lo, Second generation noninvasive fetal genome analysis reveals de novo mutations, single-base parental inheritance, and preferred DNA ends. Proc Natl Acad Sci USA 113, E8159-E8168 (2016).
26. G. X. Zheng, J. M. Terry, P. Belgrader, P. Ryvkin, Z. W. Bent, R. Wilson, S. B. Ziraldo, T. D. Wheeler, G. P. McDermott, J. Zhu, M. T. Gregory, J. Shuga, L. Montesclaros, J. G. Underwood, D. A. Masquelier, S. Y. Nishimura, M. Schnall-Levin, P. W. Wyatt, C. M. Hindson, R. Bharadwaj, A. Wong, K. D. Ness, L. W. Beppu, H. J. Deeg, C. McFarland, K. R. Loeb, W. J. Valente, N. G. Ericson, E. A. Stevens, J. P. Radich, T. S. Mikkelsen, B. J. Hindson, J. H. Bielas, Massively parallel digital transcriptional profiling of single cells. Nat Commun 8, 14049 (2017).
27. S. Kovats, E. K. Main, C. Librach, M. Stubblebine, S. J. Fisher, R. DeMars, A class I antigen, HLA-G, expressed in human trophoblasts. Science 248, 220-223 (1990).
28. S. Djurisic, T. V. Hviid, HLA Class Ib Molecules and Immune Cells in Pregnancy and Preeclampsia. Front Immunol 5, 652 (2014).
29. J. Trowsdale, A. Moffett, N K receptor interactions with MHC class I molecules in pregnancy. Semin Immunol 20, 317-320 (2008).
30. R. Sood, J. L. Zehnder, M. L. Druzin, P. O. Brown, Gene expression patterns in human placenta. Proc Natl Acad Sci USA 103, 5478-5483 (2006).
31. C. Trapnell, D. Cacchiarelli, J. Grimsby, P. Pokharel, S. Li, M. Morse, N. J. Lennon, K. J. Livak, T. S. Mikkelsen, J. L. Rinn, The dynamics and regulators of cell fate decisions are revealed by pseudotemporal ordering of single cells. Nat Biotechnol 32, 381-386 (2014).
32. S. Mi, X. Lee, X. P. Li, G. M. Veldman, H. Finnerty, L. Racie, E. LaVallie, X. Y. Tang, P. Edouard, S. Howes, J. C. Keith, J. M. McCoy, Syncytin is a captive retroviral envelope protein involved in human placental morphogenesis. Nature 403, 785-789 (2000).
33. J. Sugimoto, M. Sugimoto, H. Bernstein, Y. Jinno, D. Schust, A novel human endogenous retroviral protein inhibits cell-cell fusion. Sci Rep 3, 1462 (2013).
34. E. K. Ng, N. B. Tsui, T. K. Lau, T. N. Leung, R. W. Chiu, N. S. Panesar, L. C. Lit, K. W. Chan, Y. M. Lo, mRNA of placental origin is readily detectable in maternal plasma. Proc Natl Acad Sci USA 100, 4748-4753 (2003).
35. M. N. Cabili, C. Trapnell, L. Goff, M. Koziol, B. Tazon-Vega, A. Regev, J. L. Rinn, Integrative annotation of human large intergenic noncoding RNAs reveals global properties and specific subclasses. Genes Dev 25, 1915-1927 (2011).
36. H. Valdimarsson, C. Mulholland, V. Fridriksdottir, D. V. Coleman, A longitudinal study of leucocyte blood counts and lymphocyte responses in pregnancy: a marked early increase of monocyte-lymphocyte ratio. Clin Exp Immunol 53, 437-443 (1983).
37. M. Watanabe, Y. Iwatani, T. Kaneda, Y. Hidaka, N. Mitsuda, Y. Morimoto, N. Amino, Changes in T, B, and NK lymphocyte subsets during and after normal pregnancy. Am J Reprod Immunol 37, 368-377 (1997).
38. J. Lima, C. Martins, M. J. Leandro, G. Nunes, M. J. Sousa, J. C. Branco, L. M. Borrego, Characterization of B cells in healthy pregnant women from late pregnancy to postpartum: a prospective observational study. BMC Pregnancy Childbirth 16, 139 (2016).
39. W. C. Andrews, R. W. Bonsnes, The leucocytes during pregnancy. Am J Obstet Gynecol 61, 1129-1135 (1951).
40. R. M. Pitkin, D. L. Witte, Platelet and leukocyte counts in pregnancy. JAMA 242, 2696-2698 (1979).
41. A. J. Balloch, M. N. Cauchi, Reference ranges for haematology parameters in pregnancy derived from patient populations. Clin Lab Haematol 15, 7-14 (1993).
42. P. Brennecke, S. Anders, J. K. Kim, A. A. Kolodziejczyk, X. Zhang, V. Proserpio, B. Baying, V. Benes, S. A. Teichmann, J. C. Marioni, M. G. Heisler, Accounting for technical noise in single-cell RNA-seq experiments. Nat Methods 10, 1093-1095 (2013).
43. A. A. Kolodziejczyk, J. K. Kim, J. C. Tsang, T. Ilicic, J. Henriksson, K. N. Natarajan, A. C. Tuck, X. Gao, M. Buhler, P. Liu, J. C. Marioni, S. A. Teichmann, Single Cell RNA-Sequencing of Pluripotent States Unlocks Modular Transcriptional Variation. Cell Stem Cell 17, 471-485 (2015).
44. E. DiFederico, O. Genbacev, S. J. Fisher, Preeclampsia is associated with widespread apoptosis of placental cytotrophoblasts within the uterine wall. Am J Pathol 155, 293-301 (1999).
45. F. Reister, H. G. Frank, J. C. Kingdom, W. Heyl, P. Kaufmann, W. Rath, B. Huppertz, Macrophage-induced apoptosis limits endovascular trophoblast invasion in the uterine wall of preeclamptic women. Lab Invest 81, 1143-1152 (2001).
46. D. N. Leung, S. C. Smith, K. F. To, D. S. Sahota, P. N. Baker, Increased placental apoptosis in pregnancies complicated by preeclampsia. Am J Obstet Gynecol 184, 1249-1250 (2001).
47. N. Ishihara, H. Matsuo, H. Murakoshi, J. B. Laoag-Fernandez, T. Samoto, T. Maruo, Increased apoptosis in the syncytiotrophoblast in human term placentas complicated by either preeclampsia or intrauterine growth retardation. American Journal of Obstetrics and Gynecology 186, 158-166 (2002).
48. P. K. Lala, C. Chakraborty, Factors regulating trophoblast migration and invasiveness: possible derangements contributing to pre-eclampsia and fetal injury. Placenta 24, 575-587 (2003).
49. M. Kadyrov, J. C. Kingdom, B. Huppertz, Divergent trophoblast invasion and apoptosis in placental bed spiral arteries from pregnancies complicated by maternal anemia and early-onset preeclampsia/intrauterine growth restriction. Am J Obstet Gynecol 194, 557-563 (2006).
50. S. Z. Tomas, I. K. Prusac, D. Roje, I. Tadin, Trophoblast apoptosis in placentas from pregnancies complicated by preeclampsia. Gynecol Obstet Invest 71, 250-255 (2011).
51. M. S. Longtine, B. Chen, A. O. Odibo, Y. Zhong, D. M. Nelson, Villous trophoblast apoptosis is elevated and restricted to cytotrophoblasts in pregnancies complicated by preeclampsia, IUGR, or preeclampsia with IUGR. Placenta 33, 352-359 (2012).
52. Y. M. Lo, K. C. Chan, H. Sun, E. Z. Chen, P. Jiang, F. M. Lun, Y. W. Zheng, T. Y. Leung, T. K. Lau, C. R. Cantor, R. W. Chiu, Maternal plasma DNA sequencing reveals the genome-wide genetic and mutational profile of the fetus. Sci Transl Med 2, 61ra91 (2010).
53. W. W. Hui, P. Jiang, Y. K. Tong, W. S. Lee, Y. K. Cheng, M. I. New, R. A. Kadir, K. C. Chan, T. Y. Leung, Y. M. Lo, R. W. Chiu, Universal Haplotype-Based Noninvasive Prenatal Testing for Single Gene Diseases. Clin Chem 63, 513-524 (2017).
54. M. Pavlicev, G. P. Wagner, A. R. Chavan, K. Owens, J. Maziarz, C. Dunn-Fletcher, S. G. Kallapur, L. Muglia, H. Jones, Single-cell transcriptomics of the human placenta: inferring the cell communication network of the maternal-fetal interface. Genome Res, (2017).
55. L. Ji, J. Brkic, M. Liu, G. Fu, C. Peng, Y. L. Wang, Placental trophoblast cell differentiation: physiological regulation and pathological relevance to preeclampsia. Mol Aspects Med 34, 981-1023 (2013).
56. E. Z. Macosko, A. Basu, R. Satija, J. Nemesh, K. Shekhar, M. Goldman, I. Tirosh, A. R. Bialas, N. Kamitaki, E. M. Martersteck, J. J. Trombetta, D. A. Weitz, J. R. Sanes, A. K. Shalek, A. Regev, S. A. McCarroll, Highly Parallel Genome-wide Expression Profiling of Individual Cells Using Nanoliter Droplets. Cell 161, 1202-1214 (2015).
57. A. M. Klein, L. Mazutis, I. Akartuna, N. Tallapragada, A. Veres, V. Li, L. Peshkin, D. A. Weitz, M. W. Kirschner, Droplet barcoding for single-cell transcriptomics applied to embryonic stem cells. Cell 161, 1187-1201 (2015).
58. T. M. Gierahn, M. H. Wadsworth, 2nd, T. K. Hughes, B. D. Bryson, A. Butler, R. Satija, S. Fortune, J. C. Love, A. K. Shalek, Seq-Well: portable, low-cost RNA sequencing of single cells at high throughput. Nat Methods, (2017).
59. A. Dobin, C. A. Davis, F. Schlesinger, J. Drenkow, C. Zaleski, S. Jha, P. Batut, M. Chaisson, T. R. Gingeras, STAR: ultrafast universal RNA-seq aligner. Bioinformatics 29, 15-21 (2013).
60. M. I. Love, W. Huber, S. Anders, Moderated estimation of fold change and dispersion for RNA-seq data with DESeq2. Genome Biol 15, 550 (2014).
61. Pang W W, et al. (2009) A strategy for identifying circulating placental RNA markers for fetal growth assessment. Prenat Dia 29(5):495-504.
62. Muraro M J, et al. (2016) A Single-Cell Transcriptome Atlas of the Human Pancreas. Cell Syst 3(4):385-394 e383.
63. Zeisel A, et al. (2015) Brain structure. Cell types in the mouse cortex and hippocampus revealed by single-cell RNA-seq. Science 347(6226):1138-1142.
64. Patel A P, et al. (2014) Single-cell RNA-seq highlights intratumoral heterogeneity in primary glioblastoma. Science 344(6190):1396-1401.
65. Ng E K, et al. (2002) Presence of filterable and nonfilterable mRNA in the plasma of cancer patients and healthy individuals. Clin Chem 48(8):1212-1217.
66. Wong B C, et al. (2005) Circulating placental RNA in maternal plasma is associated with a preponderance of 5′ mRNA fragments: implications for noninvasive prenatal diagnosis and monitoring. Clin Chem 51(10):1786-1795.
67. Chiu R W, et al. (2005) Fetal rhesus D mRNA is not detectable in maternal plasma. Clin Chem 51(11):2210-2211.
68. Sanz I (2014) Rationale for B cell targeting in SLE. Semin Immunopathol 36(3):365-375.
69. Chan R W, Wong J, Lai P B, Lo Y M, Chiu R W. The potential clinical utility of serial plasma albumin mRNA monitoring for the post-liver transplantation management. Clin Biochem. 2013; 46(15):1313-9.
70. Chan R W, Wong J, Chan H L, Mok T S, Lo W Y, Lee V, et al. Aberrant concentrations of liver-derived plasma albumin mRNA in liver pathologies. Clin Chem. 2010; 56(1):82-9.

What is claimed is:

1. A method of identifying an expressed marker to differentiate between different levels of a condition, the method comprising:
for each cell of a plurality of cells obtained from one or more first subjects:
analyzing RNA molecules from the cell to obtain a set of reads, thereby obtaining a plurality of sets of reads;
for each read of the set of reads:
identifying, by a computer system, an expressed region in a reference sequence corresponding to the read;
for each of a plurality of expressed regions:
determining an amount of reads corresponding to the expressed region;
determining an expression score for the expressed region using the amount of reads corresponding to the region, thereby determining a multidimensional expression point comprised of the expression scores for the plurality of expressed regions;
grouping, by the computer system, the plurality of cells into a plurality of clusters using the multidimensional expression points corresponding to the plurality of cells, the plurality of clusters being less than the plurality of cells;
for each cluster of the plurality of clusters, determining a set of one or more preferentially expressed regions that are expressed in cells of the cluster at a specified rate more than cells of other clusters;
for each of a plurality of cell-free RNA samples:
analyzing a plurality of cell-free RNA molecules to obtain a plurality of cell-free reads, wherein the plurality of cell-free RNA samples are from a plurality of cohorts of second subjects, wherein each cohort of the plurality of cohorts has a different level of the condition; and
for each set of one or more preferentially expressed regions of the plurality of sets of one or more preferentially expressed regions:
measuring a signature score for the corresponding cluster using cell-free reads corresponding to the set of one or more preferentially expressed regions;
identifying, based on the signature scores, one or more of the sets of one or more preferentially expressed regions as one or more expressed markers for use in classifying future samples to differentiate between different levels of the condition.

2. The method of claim 1, wherein:
the condition is a pregnancy-associated condition,
the first subjects are female subjects each pregnant with a fetus,
the plurality of cells are placental cells,
the second subjects are female subjects each pregnant with a fetus.

3. The method of claim 2, wherein the cell-free RNA samples are obtained from plasma or serum of the second subjects.

4. The method of claim 2, wherein the pregnancy-associated condition is preeclampsia.

5. The method of claim 4, wherein the levels are severities of preeclampsia.

6. The method of claim 4, wherein:
each cohort includes sub-cohorts that have different gestational ages, and
a first set of one or more preferentially expressed regions is a first expressed marker that differentiates between different levels of the condition for a first gestational age.

7. The method of claim 1, wherein the condition is cancer.

8. The method of claim 7, wherein the levels of the condition are whether cancer exists, different stages of cancer, different sizes of tumor, the cancer's responses to treatment, or another measure of a severity or progression of cancer.

9. The method of claim 7, wherein a first set of one or more preferentially expressed regions of a first cluster of the plurality of clusters is a first expressed marker that differentiates between levels of cancer for a first tissue, wherein the first cluster includes cells from the first tissue.

10. The method of claim 9, wherein:
the first tissue is from the liver, thereby having the first cluster including liver cells;
the liver cells comprise tumor cells and non-tumor cells or the liver cells do not comprise tumor cells, and
the cancer is hepatocellular carcinoma.

11. The method of claim 1, wherein:
the condition is systemic lupus erythematosus (SLE), and
the plurality of cells are kidney cells.

12. The method of claim 1, further comprising:
for each cell of the plurality of cells:
storing, in a memory of the computer system, the set of reads associated with a unique code corresponding to the cell,
wherein identifying the expressed region in the reference sequence corresponding to the read includes performing an alignment procedure using the read and a plurality of expressed regions of the reference sequence, and
wherein determining the amount of reads corresponding to a first expressed region of a first cell of the plurality of cells uses (1) the unique code corresponding to the first cell so as to identify reads corresponding to the first cell and (2) results of the alignment procedure for the set of reads of the first cell.

13. The method of claim 1, further comprising:
obtaining a sample comprising the plurality of cells;
isolating each cell of the plurality of cells to enable analyzing the RNA molecules of a particular cell.

14. The method of claim 13, further comprising:
tagging RNA molecules of each cell of the plurality of cells with a unique code for the cell such that the associated reads include the unique code and
storing, in a memory of the computer system, each set of reads associated with the unique code of the cell corresponding to the set of reads.

15. The method of claim 1, wherein:
the specified rate comprises a value determined from an average expression score for cells of the cluster and an average expression score for cells of other clusters.

16. The method of claim 1, wherein:
grouping the plurality of cells into the plurality of clusters comprises performing dimensionality-reduction methods or by using force-based methods on the multidimensional expression points

17. The method of claim 16, wherein:
grouping the plurality of cells into the plurality of clusters comprises performing dimensionality-reduction methods, and
the dimensionality-reduction methods comprise principal component analysis (PCA) or diffusion maps.

18. The method of claim 16, wherein:
grouping the plurality of cells into the plurality of clusters comprises using force-based methods, and
the force-based methods comprise t-distributed stochastic neighbor embedding (t-SNE).

19. The method of claim 1, further comprising:
identifying a first cluster of the plurality of clusters to include a first type of cell by comparing the set of one or more preferentially expressed regions of the first cluster with one or more regions known to be preferentially expressed in the first type of cell.

20. The method of claim 19, wherein the first type of cell comprises decidual, endothelial, vascular smooth muscle, stromal, dendritic, Hofbauer, T, erythroblast, extravillous trophobast, cytotrophoblast, syncytiotrophoblast, B, monocyte, hepatocyte-like, cholangiocyte-like, myofibroblast-like, endothelial, lymphoid, or myeloid cells.

21. The method of claim 1, wherein the first subjects are the same as the second subjects.

22. The method of claim 1, wherein the signature score is an average of an expression level for the preferentially expressed region for the corresponding cluster.

23. The method of claim 1, wherein identifying one or more of the sets of one or more preferentially expressed regions for use in classifying future samples to differentiate between different levels of the condition comprises identifying a signature score for a cohort and for a cluster that is statistically different than the signature scores for other cohorts in the cluster.

24. The method of claim 1, further comprising:
receiving a plurality of cell-free reads from an analysis of cell-free RNA molecules from a biological sample obtained from a third subject;
for each preferentially expressed region of a first expressed marker:
determining an amount of reads for the preferentially expressed region, and
comparing the amount of reads for one or more preferentially expressed regions to one or more reference values; and
determining, based on the comparison of the amount of reads for one or more preferentially expressed regions to one or more reference values, a level of the condition for the third subject.

25. The method of claim 24, further comprising:
analyzing a plurality of cell-free RNA molecules from the biological sample obtained from the third subject to obtain a plurality of cell-free reads.

26. The method of claim 24, wherein comparing the amount of reads for one or more preferentially expressed regions to one or more reference values comprises comparing the amount of reads for each preferentially expressed region to a reference value for each preferentially expressed region.

27. The method of claim 24, wherein comparing the amount of reads for one or more preferentially expressed regions to one or more reference values comprises:
calculating an overall score from the amount of reads for one or more preferentially expressed regions, and
comparing the overall score to one reference value.

28. A method of determining a level of a condition in a subject, the method comprising:
receiving a plurality of cell-free reads from analysis of cell-free RNA molecules from a biological sample obtained from the subject;
for each preferentially expressed region of one or more expressed markers, the one or more expressed markers determined by the method of claim 1:
determining an amount of reads for the preferentially expressed region, and
comparing the amount of reads to a reference value for one or more preferentially expressed regions to one or more reference values; and
determining, based on the comparisons of the amount of reads for each preferentially expressed regions to one or more reference values, the level of the condition for the subject.

29. A method of determining a level of a condition in a subject, the method comprising:
receiving a plurality of cell-free reads from analysis of cell-free RNA molecules from a biological sample obtained from the subject;
determining a value of a temporal parameter related to the condition;
determining, using the value of the temporal parameter, an expressed markers for the condition at a time of the value of the temporal parameter, the expressed marker comprising one or more sets of preferentially expressed regions;
for each preferentially expressed region of the expressed marker:
determining an amount of reads corresponding to the preferentially expressed region;
comparing the amount of reads for one or more preferentially expressed regions to one or more reference values; and
determining, based on the comparison of the amount of reads for one or more preferentially expressed regions to one or more reference values, the level of the condition for the subject.

30. The method of claim 29, wherein:
the condition is a pregnancy-associated condition, and
the subject is a female pregnant with a fetus.

31. The method of claim 30, wherein the pregnancy-associated condition is preeclampsia.

32. The method of claim 30, wherein the temporal parameter is gestational age expressed as a week of pregnancy, a month of pregnancy, or a trimester of pregnancy.

33. The method of claim 30, wherein the condition is cancer.

34. The method of claim 33, wherein the temporal parameter is a duration of treatment, a time since diagnosis of cancer, or post-operative survival time.

35. The method of claim 29, wherein comparing the amount of reads for one or more preferentially expressed regions to one or more reference values comprises comparing the amount of reads for each preferentially expressed region to a reference value for each preferentially expressed region.

36. The method of claim 29, wherein comparing the amount of reads for one or more preferentially expressed regions to one or more reference values comprises:
calculating an overall score from the amount of reads for one or more preferentially expressed regions, and
comparing the overall score to one reference value.

37. A computer product comprising a computer readable medium storing a plurality of instructions for controlling a computer system to perform the method of claim 1.

38. A system comprising one or more processors configured to perform the method of claim 1.
